(19) United States
(12) Patent Application Publication
(10) Pub. No.: US 2012/0164649 A1
(43) Pub. Date: Jun. 28, 2012

(54) SYSTEM, DEVICES AND METHODS FOR MONITORING AND DETECTION OF CHEMICAL REACTIONS

(75) Inventors: Rick T. SMETHERS, Malvern, PA (US); Justin Dale Brubaker, Lafayette Hill, PA (US); Thomas Vincent Connors, Audubon, PA (US); Michael Jay Fischer, Jenkintown, PA (US); Jeffery J. Guterman, Los Angeles, CA (US); Vincent A. Juliana, Exton, PA (US); Hans C. Kastensmith, Aldie, VA (US); James F. Koller, Downingtown, PA (US); David J. Lyons, W. Trenton, NJ (US); Daniel Arthur Miller, Bryn Mawr, PA (US)

(73) Assignee: INSTANTLABS MEDICAL DIAGNOSTICS CORPORATION, Washington, DC (US)

(21) Appl. No.: 13/204,291

(22) Filed: Aug. 5, 2011

Related U.S. Application Data
(60) Provisional application No. 61/371,446, filed on Aug. 6, 2010.

(51) Int. Cl. C12Q 1/68; C12M 1/34

(52) U.S. Cl. 435/6.12; 435/287.2

(57) ABSTRACT

Systems, devices and methods are described herein that are configured for use in the monitoring and detecting of chemical reactions, such as, for example, the monitoring and detection of Polymerase chain reactions (PCR). For example, the systems and devices described herein can be used for accelerated real-time PCR. A fully integrated PCR system is provided that includes a touch screen user interface, eliminating the need for additional computers, keyboards, and related devices. The PCR systems described herein can be network enabled to provide communications between one or more PCR monitoring and detection devices and a central monitoring station. A disposable sample holding device can be placed in the PCR device for testing in an upright vertical orientation, providing improved optical scanning capabilities and rapid heating and cooling capabilities.

### CROSS-REFERENCE TO RELATED APPLICATIONS

This application claims priority to and the benefit of U.S. Provisional Patent Application Ser. No. 61/371,446, filed Aug. 6, 2010, entitled “System, Devices and Methods for Monitoring and Detection of Chemical Reactions,” the disclosure of which is hereby incorporated herein by reference in its entirety.

### BACKGROUND

The invention relates generally to systems, devices and methods for monitoring and detection of chemical and/or bio-chemical reactions, including, for example, the monitoring and detection of Polymerase Chain Reactions (PCR).

Analytical processes that only require small amounts of DNA have many applications in various fields, such as microbiology, forensics, food science, bio-defense, and water purification. Another application of such processes is for pre-implantation genetic diagnosis (PGD) where there is only one cell with which to work and from which to extract DNA. PGD can also require an answer quickly so that the embryos can be selected for transfer without having to freeze (store) them.

PCR is a valuable technique because the reaction is highly specific, and capable of creating large amounts of copied DNA fragments from minute amounts of samples for both sequencing and genotyping applications. For this reason, PCR has wide applications in clinical medicine, genetic disease diagnostics, forensic science, and evolutionary biology. Recently, miniaturized PCR devices have attracted great interest because they have many advantages over conventional PCR devices, such as, portability, higher thermal cycling speed, and significantly reduced reagents/sample consumption. Most mini/micro PCR devices can be classified into two types, static chamber PCR chips and dynamic flow PCR chips. The first type of device uses stationary thermal cyclers to heat and cool a static volume of liquid in a micro-chamber. In these devices, either the micro-chamber is manufactured separately and placed in contact with an external heater, or the micro-chamber and the micro-heater are bonded together to form a complete microchip. The second type of device, a dynamic flow-through PCR device, heats and cools PCR reactants by flowing the reactants through different temperature zones. A typical flow-through thermal cycler is one with thin film platinum heaters and sensors patterned onto a silicon wafer to generate three different temperature zones. In this second type of PCR system, it can be difficult to examine the PCR results and to collect the PCR product. Reliability of this type of device cannot be assured unless reliable pumping and inter-channel connection are available at an acceptable cost.

Research has also been done towards integrating PCR with either pre-PCR or post PCR processes to further utilize the advantages of microfluidics. Real-time PCR, as it is known, is highly attractive because it can detect and quantify PCR results through real-time analysis of fluorescent signals generated during the reaction, without the conventional post-PCR processes, such as, gel electrophoresis. While real-time PCR has significant advantages compared to regular PCR, there can be limitations to the application of real-time PCR techniques. For example, during real-time PCR, the optical detection system must monitor the fluorescence intensity in real-time. At least two separate sets of excitation-detection wavelength pairs must be available at each PCR well to identify both the desired species and control species in each well. As the number of wells and/or desired light interaction increases, the optical infrastructure can grow greatly, increasing the complexity, cost, and size of the optical detection module.

Currently, many of the instruments for conducting real-time PCR are bulky and expensive. In addition, existing systems require the use of external computers to monitor and record the testing data. Thus, there is a need for improved systems that are smaller, less expensive and self-contained.

### SUMMARY OF THE INVENTION

Systems, devices and methods are described herein that are configured for use in the monitoring and detection of chemical reactions, such as, for example, the monitoring and detection of PCR. For example, the systems and devices described herein can be used for accelerated real-time PCR. A fully integrated PCR system is provided that includes a touch screen user interface, eliminating the need for additional computers, keyboards, and related devices. The PCR systems described herein can be network enabled to provide communications between one or more PCR monitoring and detection devices and a central monitoring station. A disposable sample holding device can be placed in the PCR device for testing in an upright, vertical orientation, providing improved optical scanning capabilities and rapid heating and cooling capabilities.

### BRIEF DESCRIPTION OF THE DRAWINGS

FIG. 1 is a schematic illustration of a system according to an embodiment.

FIG. 2 is a schematic illustration of a system according to an embodiment.

FIG. 3 is a front view of a computer monitor of the central monitoring station of FIG. 2.

FIG. 4 is a schematic of a portion of a PCR monitoring and detection device according to an embodiment.

FIG. 5 is a front view of a PCR monitoring and detection device according to an embodiment.

FIG. 6 is a front view of the PCR monitoring and detection device of FIG. 5 and a disposable cartridge according to an embodiment.

FIG. 7 is a front view of a portion of the PCR monitoring and detection device of and disposable cartridge of FIG. 5.

FIG. 8 is a front view of the disposable cartridge of FIG. 6.

FIG. 9 is an illustration of a portion of the PCR monitoring and detection device of FIG. 5 scanning a bar code on the cartridge of FIG. 8.

FIG. 10a is a perspective view of an illustration of a portion of the PCR monitoring and detection device of FIG. 5 illustrating an optical scanning device scanning a test sample of a disposable cartridge.

FIG. 10b is a side view of an illustration of a portion of the PCR monitoring and detection device of FIG. 5 illustrating an optical scanning device, a disposable cartridge and a thermal cycler.

FIG. 10c is a schematic illustration of a portion of the optical scanning device of the PCR monitoring and detection device of FIG. 5.

FIG. 10d is a top view of a schematic illustration of a portion of the PCR monitoring and detection device of FIG. 5.

FIG. 11 is a front perspective view of a disposable cartridge according to an embodiment.

FIG. 12 is a front perspective view of a portion of the disposable cartridge of FIG. 11.

FIG. 13 is a front elevational view of a portion of the disposable cartridge of FIG. 11.

FIG. 14 is a front elevational view of base member of the disposable cartridge of FIG. 11.

FIG. 15 is a front elevational view of a portion of the base member of FIG. 11.

FIG. 16 is a side view of the base member of FIG. 11.

FIG. 17 is front perspective view of a cover member of the disposable cartridge of FIG. 11.

FIG. 18 is side view of the cover member of FIG. 17.

FIG. 19 is a front elevational view of the cover member of FIG. 17 and a portion of the base member of FIG. 15.

FIG. 20 is a side view of the cover member and portion of the base member of FIG. 19.

FIG. 21 is a front view of a cap member of the disposable cartridge of FIG. 11.

FIG. 22 is a rear perspective view of a portion of the cap member of FIG. 21.

FIG. 23 is a side view of a portion of the disposable cartridge of FIG. 11.

FIG. 24 is a side view of a portion of the disposable cartridge of FIG. 11.

FIGS. 25-37 are example screen shots from a touch screen user interface according to an embodiment.

FIG. 38a is a front perspective view of a portion of a disposable cartridge according to an embodiment; and FIG. 38b is a front perspective view of a portion of the disposable cartridge of FIG. 38a.

FIG. 39a is a perspective view showing a cross-section of a portion of the disposable cartridge of FIG. 38a; and FIG. 39b is a side elevational cross-sectional view of a portion of the disposable cartridge of FIG. 38a.

FIG. 40 is a cross-sectional view the portion of the disposable cartridge of FIG. 38b and a test sample insertion device.

FIG. 41 is an illustration of a portion of a PCR monitoring and detection device and an optical scanning device scanning a test sample of a disposable cartridge according to an embodiment.

FIG. 42a-42c each illustrate a portion of a disposable cartridge according to another embodiment.

FIG. 43 is a table listing example organisms that can be provided pre-loaded in a disposable cartridge.

FIG. 44 is a graph illustrating an example thermal profile.

FIG. 45 is a graph and illustration showing an example of detected emissions using a traditional detection method; and

FIG. 46 is a graph and illustration showing an example detected emissions using a Hunting method.

FIG. 47 is a schematic illustration of a portion of an optical scanning device and a disposable cartridge.

FIG. 48a is a perspective of a portion of PCR monitoring and detection device shown with the access door in an open position; and FIG. 48b is a perspective of the portion of the PCR monitoring and detection device of FIG. 48a shown with the door in a closed position.

FIG. 49 is a front perspective view of another embodiment of a disposable cartridge.

FIG. 50a is rear perspective view of a housing according to an embodiment; and FIG. 50b is a front perspective of the housing of FIG. 50a.

FIG. 51 is a rear perspective view of a PCR monitoring and detection device according to another embodiment.

FIG. 52 is a perspective view of a disposable cartridge according to another embodiment.

FIG. 53 is a perspective view of a portion of the disposable cartridge of FIG. 52.

FIG. 54 is a front elevational view of the portion of the disposable cartridge of FIG. 52.

FIG. 55 is a perspective view of a cover of the disposable cartridge of FIG. 52.

FIG. 56 is a perspective view of a cap member of the disposable cartridge of FIG. 52.

FIG. 57 is a side view of an illustration of a portion of a PCR monitoring and detection device, illustrating an optical scanning device, a disposable cartridge and a thermal cycler.

FIG. 58 is a perspective view of a portion of the PCR monitoring and detection device of FIG. 57.

FIG. 59 is a front perspective view of another embodiment of a disposable cartridge.

FIG. 60 is a front perspective view of a PCR device according to an embodiment.

FIG. 61 is a front perspective view of a schematic illustration of a portion of a PCR device according to an embodiment.

FIG. 62 is schematic illustration of a system according to another embodiment.

FIG. 63 is a schematic systems diagram illustrating the operative connections between the various components of a PCR device according to an embodiment.

FIG. 64 is a perspective view of a disposable cartridge according to another embodiment, shown with the cap members in a closed position.

FIG. 65 is a perspective view of the disposable cartridge of FIG. 64, shown with the cap members in an open position.

FIG. 66 is a front view of a base member of the disposable cartridge of FIG. 64.

FIG. 67 is front view of a portion of the base member of FIG. 66.

FIG. 68 is a sectional view of the base member of FIG. 64, taken along line 68-68 in FIG. 64.

FIG. 69 is a front view of a cover member of the disposable cartridge of FIG. 64.

FIG. 70 is a back view of the cover member of FIG. 70.

FIG. 71 is a sectional view of the cover member of FIG. 69, taken along line 71-71 in FIG. 69.

FIG. 72 is a perspective view of the cover member of FIG. 69.

FIG. 73 is a perspective view of a cap member of he disposable cartridge of FIG. 64.

FIG. 74 is front view of a portion of the cartridge assembly of FIG. 64.

FIG. 75 is a sectional view of the portion of the cartridge assembly of FIG. 74, taken along line 75-75 in FIG. 74.

### DETAILED DESCRIPTION

Systems, devices and methods are described herein that are configured for use in the monitoring and detection of chemical reactions, such as, for example, the monitoring and detection of PCR. For example, the systems and devices described herein can be used for accelerated real-time PCR. Example embodiments of a PCR system are also described in U.S. Pat. No. 7,569,382 (“the '382 patent”), the entire disclosure of which is incorporated herein by reference in its entirety. The PCR system described herein can provide a fully integrated accelerated PCR system that is lightweight, portable and inexpensive.

The PCR device is a non-invasive diagnostic testing device. It can analyze DNA of test samples based on detection of a fluorescent signal produced proportionally during the amplification of a specific DNA sequence. In general, applications that involve detecting gene mutations, detecting bacteria and viruses, performing genetic testing, or the like, can be performed using the systems and devices described herein. These applications can be found in the fields of microbiology, forensics, food science, water purification, etc. For the purpose of this description, the systems and devices will be described specifically with respect to PCR, but should not be limited to that application. The systems and devices can be used with other various applications, such as Enzyme Linked Immuno Sorbent Assay (ELISA), which is a sensitive immunoassay that uses an enzyme linked to an antibody or antigen as a marker for the detection of a specific protein, especially an antigen or antibody. The systems and methods can also be used as a diagnostic test to determine exposure to a particular infectious agent, such as the AIDS virus, by identifying antibodies present in a blood sample.

The PCR systems described herein can include the use of a computer or computers. As used herein, the term computer is intended to be broadly interpreted to include a variety of systems and devices including personal computers, laptop computers, mainframe computers, set top boxes, digital versatile disc (DVD) players, and the like. A computer can include for example, processors, memory components for storing data (e.g., read only memory (ROM) and/or random access memory (RAM), other storage devices, various input/output communication devices and/or modules for network interface capabilities, etc. Various functions of the PCR systems can be performed by software and/or hardware. The PCR systems can provide for streaming of data in real-time.

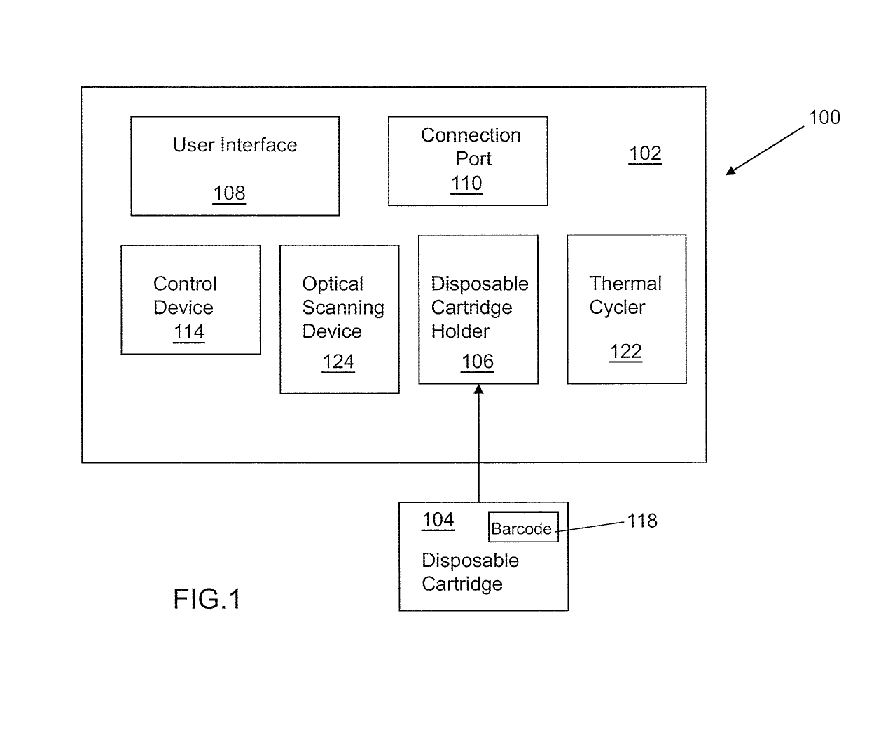

FIG. 1 is a schematic illustration of a PCR system according to an embodiment. A PCR system 100 includes a PCR monitoring and detection device 102 (also refer to herein as a PCR device) configured to receive disposable sample holders 104 containing, for example, one or more test samples. The PCR device 102 can be used to monitor the test sample(s) and detect polymerase chain reactions. The PCR system 100 can provide a fully integrated accelerated PCR system that is lightweight, portable and inexpensive.

The disposable sample holder 104 can be an insertable cartridge that can include a bar code 118, as described in more detail below. The disposable sample holder 104 can be inserted into a disposable cartridge holder 106 (also referred to herein as a cartridge enclosure or disposable cartridge enclosure) within the PCR device 102. The PCR device 102 also includes an user interface 108 that can be used to control and operate the PCR device 102. The user interface 108 can be a high resolution color touch screen display and can be icon based. Thus, the need for an external computer, keyboard and mouse can be eliminated.

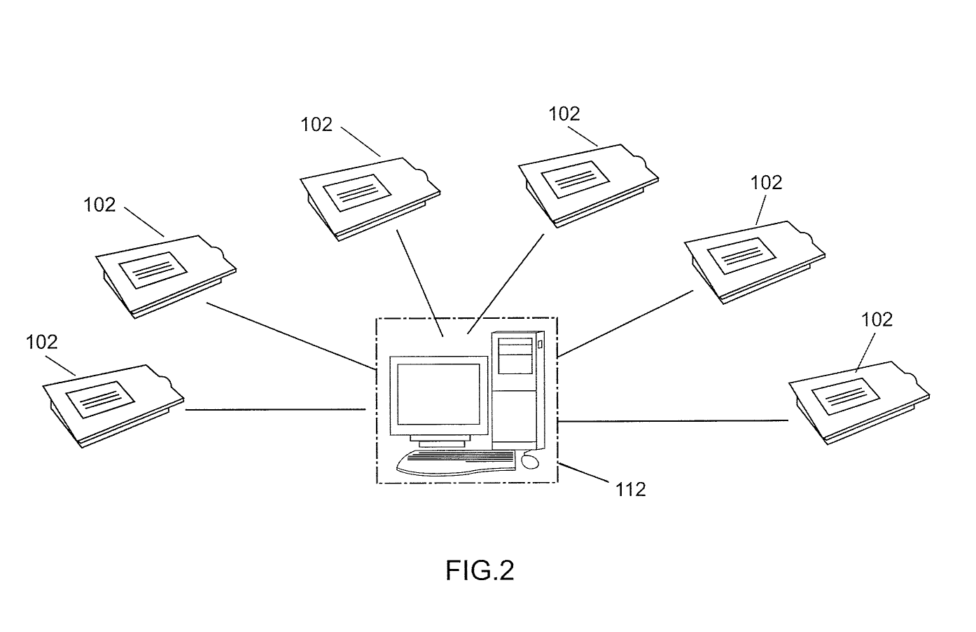

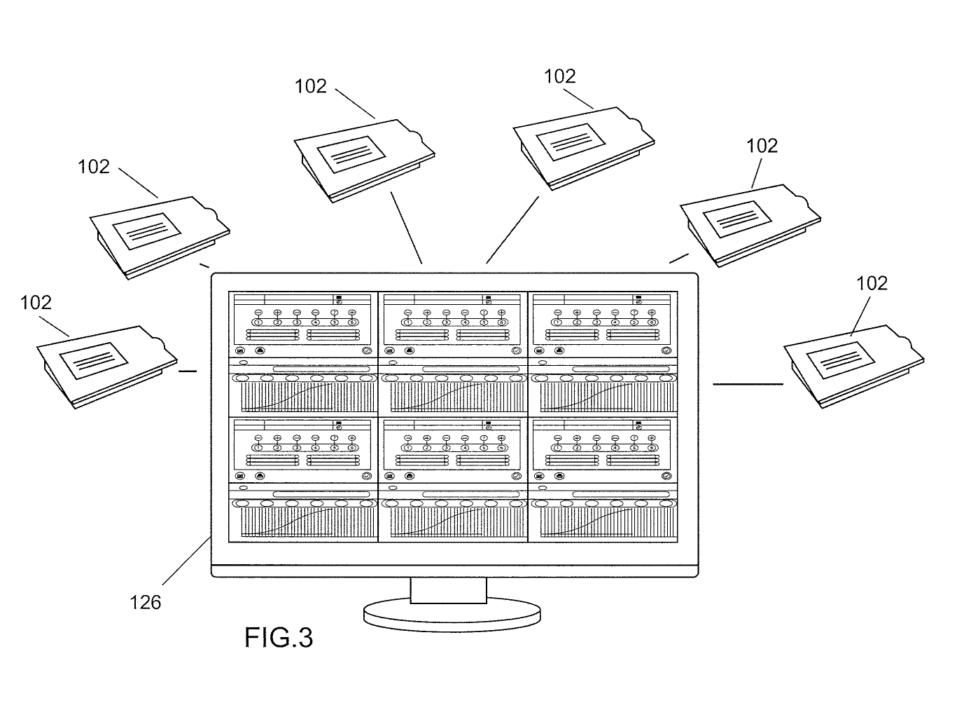

The PCR device 102 can also be configured to connect to a printer and can include a power switch (not shown). The PCR device 102 can also include a connection port 110, such as a USB port, such that the PCR device 102 can be network enabled. For example, a PCR system 100 can provide wired or wireless communication between one or multiple PCR devices 102 and a central monitoring station 112, as shown in FIG. 2. For example, a central monitoring station 112 can be used to securely view and control multiple PCR devices 102 connected via a network, such as an internal, private network, or a public network, such as the Internet. In some embodiments, a communication is provided via an internal or external server (not shown). FIG. 3 illustrates a computer monitor 126 of the central monitoring station 112 with results screens for multiple PCR devices 102. Thus, data analysis can be performed from one centralized computer. The central monitoring station 112 can provide storage of data, and software updates can be communicated between the central monitoring station 112 and one or more PCR device 102.

The PCR device 102 also includes a thermal cycler 122, an optical scanning device 124 (also referred to herein as “scanning device” or optical scanner” or “excitation device”), and a control device 114. In some embodiments, a thermal cycler can be included as described in the '382 patent incorporated by reference above. The thermal cycler 122 can be a miniature thermal cycler that can provide different temperature levels required for PCR. The thermal cycler 122 can have a heat plate (not shown in FIG. 1) that contacts the inserted disposable sample holder 104 containing the sample to be tested. The disposable sample holder 104 can be configured such that the interface between the disposable sample holder 104 and the heat plate can provide for improved heat transfer from the thermal cycler 122 to the sample volume. For example, the disposable sample holder 104 can provide a lower profile geometry at the interface such that the heat plate can be much smaller in size, and therefore, lower in mass, as compared to other PCR systems available in the art. The thermal cycler 122 can also include a thermal electric device (not shown in FIG. 1) for heating and cooling and a heat sink (not shown in FIG. 1) to maintain the side of the thermal cycler opposite of the heat plate at an optimum temperature to optimize both heating and cooling rates. The thermal cycler 122 can include one or more cooling fans (not shown in FIG. 1). The PCR device 102 can include thermistor circuits (not shown in FIG. 1) that can be positioned, for example, on the front of a heat plate and the back of the thermal cycler 122. In some embodiments, the PCR device 102 can include, for example, five thermistors. For example, in some embodiments, the PCR device 102 can include two thermistors in a heat plate, one in a heat sink, one in an ambient electronics compartment and one in an ambient thermal cycler compartment of the PCR device 102. The thermistors can be positioned to measure the temperature of the electronics compartment and to measure the temperature near the thermal cycler 122. The thermal cycler 122 can also be software controlled. The PCR device 102 can also include, for example, a stepper motor to drive the system optics, other supporting electronics, and/or other components (each not shown in FIG. 1) known in the art for such devices. The thermal cycler 122 can also include a clamping mechanism (not shown in FIG. 1) configured to securely hold the disposable sample holder 104 within the PCR device 102. The clamping mechanism can also aid in sealing the disposable sample holder 104.

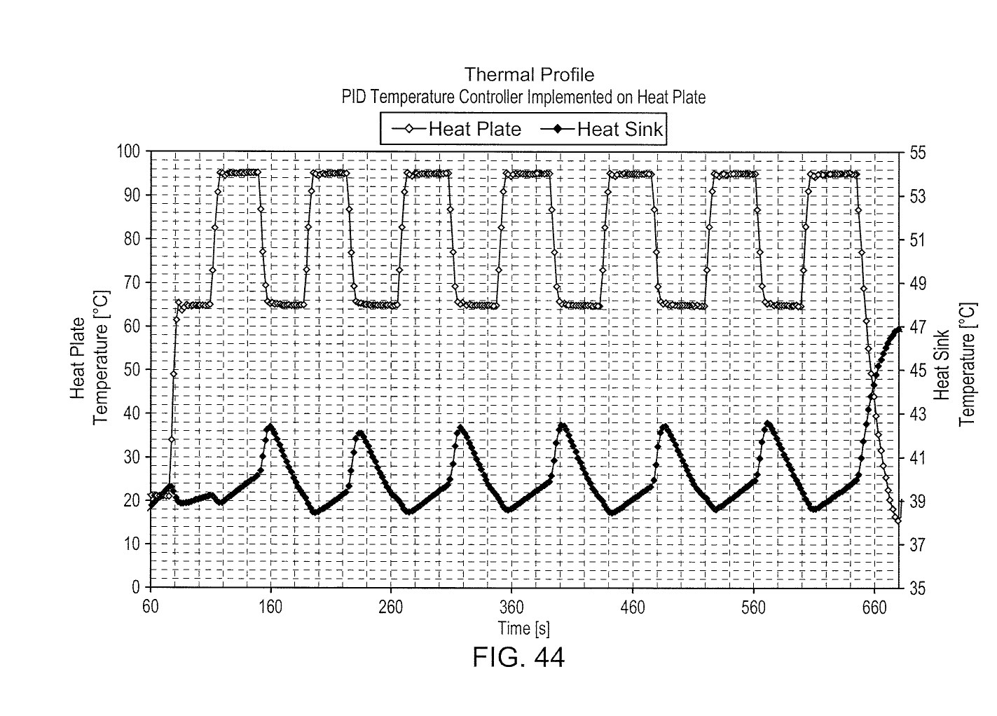

In some embodiments, the thermal cycler 122 is capable of rapid heat and cooling cycles with change rates, for example, greater than 5° C./second. Thermal accuracy of, for example, +/−0.1° C., can also be achieved. These results are typically only obtainable from much larger devices. For example, FIG. 44 illustrates an example thermal profile of temperature over time that can be achieved.

During real-time PCR, the optical scanning device 124 monitors the fluorescence intensity of the samples in real time. To accomplish this, the optical scanning device 124 can include (each not shown in FIG. 1) a light source, a detector, related optics, and a mechanism for producing relative movement of the light source, detector, and/or optics with respect to the sample holder 104. In some embodiments, the light source can be, for example, a laser. In some embodiments, the light source can be, for example, a laser diode. Power to the light source can be controlled by software, which can, for example, be configured to turn off the power when the access door is open. The optical scanning device 124 can also include a read optic (not shown in FIG. 1), such as for example, a fiber focuser. The read optic in the optical scanning device 124 can provide an optical path between the light source, the sample, and the detector. The read optic can be mounted to a carriage that translates with respect to the sample holder 104 along a linear stage with a stepper motor driven lead screw. The carriage can translate across the inserted sample holder/disposable cartridge (described in more detail below) such that the read optic (e.g., fiber focuser) will be positioned to direct the light from the light source towards the sample in each well of the cartridge and to collect light from each well and direct it towards the detector. In some embodiments, the optical scanning device 124 can be configured as described in the '382 patent incorporated by reference above.

As described above, in some embodiments, the optical scanning device 124 can be configured to move linearly across the disposable cartridge 104. The optical scanning device 124 can move the carriage (with the read optic mounted thereon) to a desired location in front of each sample well, and stop for a short time period (e.g., 1 or 2 milliseconds) to measure the fluorescence intensity of the sample. For example, the light source (e.g., laser) can work harmoniously with the movement of the carriage such that when the carriage stops for an instant in front of a sample well, the laser will activate. Thus, as the read optic moves and stops in front of the sample well, the laser is pulsed on and off and a measurement of the fluorescence can be taken. In some embodiments, the carriage is configured to stop at an edge of a sample well and will move and stop across the sample well for a designated number of times. For example, the carriage can stop 100 times as it moves across a single sample well, and each time it stops the laser is activated and a fluorescence measurement is taken. The carriage can then move to the next sample well and repeat this process. This type of movement and measurement scenario is referred to as Hunting (discussed in more detail below).

The disposable sample holder 104 (also referred to herein as “cartridge” or “disposable cartridge”) includes one or more reaction wells such that multiple individual samples can be tested for a single analyte, or multiple analytes from a single sample can be tested. For example, a single cartridge 104 can be used to run an Influenza panel that includes seasonal, avian (H5N1) and swine (H1N1) flu on a single cartridge in a single run. As mentioned above, the cartridge 104 can include a bar code 118 to identify specific assays. The cartridge 104 can then be “scanned” and read by a bar code reader scanner (not shown in FIG. 1) of the PCR device 102 to identify the test sample by a sample identification (ID). The disposable cartridge 104 can be formed with, for example, a polycarbonate or other material with suitable chemical compatibility with reagents.

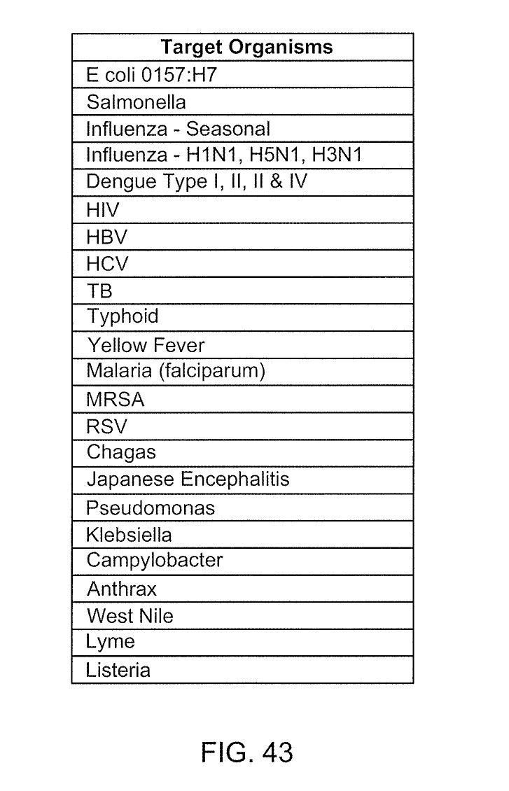

In some embodiments, a disposable cartridge 104 can be provided pre-loaded with an organism(s) to be tested. For example, FIG. 43 includes a list of example organisms that can be provided pre-loaded on a cartridge 104. In some embodiments, Lyophilized reagents can be integrated into the reaction chambers/wells to allow for easy injection of the “wet” elements of the protocol.

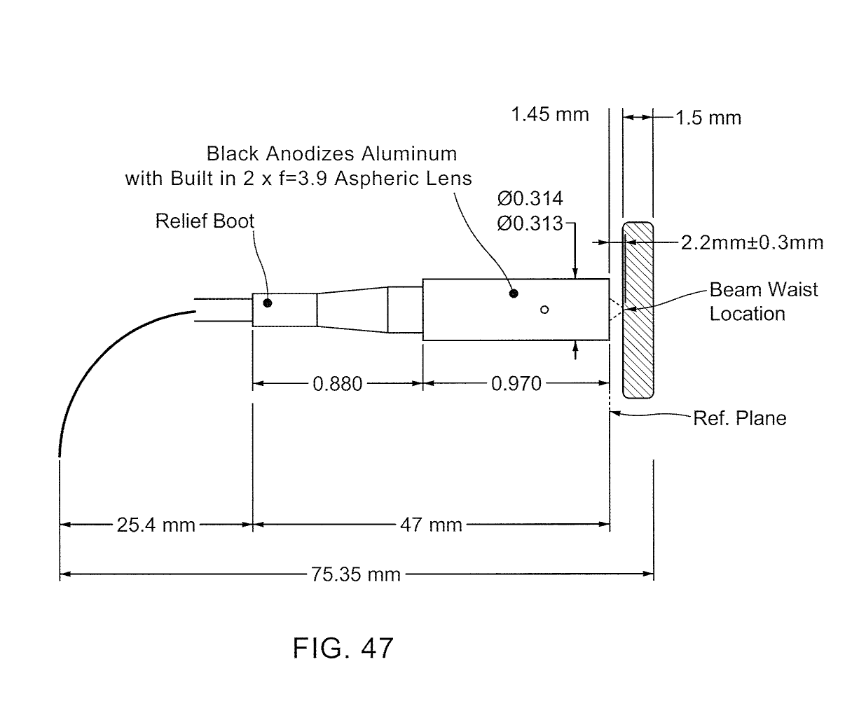

The disposable cartridge 104 is configured to be disposed within the PCR device 102 in a vertical orientation, as described in more detail below with reference to specific embodiments. The vertical orientation of the cartridge 104 provides a side-view of the reaction wells by the optical scanning device 124, and allows for optical sensing to be performed in the lower portion of the PCR reaction well(s) in a single motion/pass across the reaction wells. In other words, the disposable cartridge 104 can define a vertical axis that is parallel to an axis defined by a length of the reaction wells of the disposable cartridge 104, and a horizontal axis that is parallel to an axis defined by a width of the reaction wells. The optical scanning device 124 can translate along an axis that is substantially perpendicular to the vertical axis of the disposable cartridge 104 (or substantially parallel to the horizontal axis of the disposable cartridge 104) when the cartridge 104 is disposed within the PCR device 102 in the vertical orientation. The vertical orientation of the disposable cartridge 104 within the PCR device 102 can minimize or eliminate the likelihood of large bubbles forming in the area where the optical sensing is performed. Such bubbles can interfere with the measurement accuracy and sensitivity. FIG. 47 is a schematic illustration of a portion of an optical scanning device illustrating its positioning in relation to a reaction well of a disposable cartridge. The cartridge 104 is inserted into the PCR device 102 such that reactor wells of the cartridge 104 are positioned adjacent the thermal cycler 122. In some embodiments, the thermal cycler 122 can provide a clamping force over a cap of the cartridge 104 (described in more detail below) to help further seal the cartridge 104.

It is desirable to accomplish PCR as quickly as possible (to minimize the time to acquire results). This can be achieved by minimizing the number of temperature cycles required to produce measurable results, and by reducing the amount of time required to produce each temperature cycle for the reagents inside the PCR reaction wells. The cycle time can depend on various factors. For example, by minimizing the volume (and thus mass) of reagents, the total amount of heat required to change a set temperature difference can be minimized. The rate of heat transfer into the reagents can be increased (i.e. the conductive thermal resistance of the fluid can be reduced) by reducing the thickness, and increasing the cross-sectional area, of the reagent volume. This permits further reduction in cycle time. One consideration that limits the reduction in reagent volume thickness is avoiding capillary action filling, which can increase the time it takes for the individual to fill the reaction well. The reduced volumetric footprint also provides more area that can be scanned by the optical scanning device 104 to detect DNA. Thus, the PCR system described herein is configured for rapid thermal transfer and optical detection.

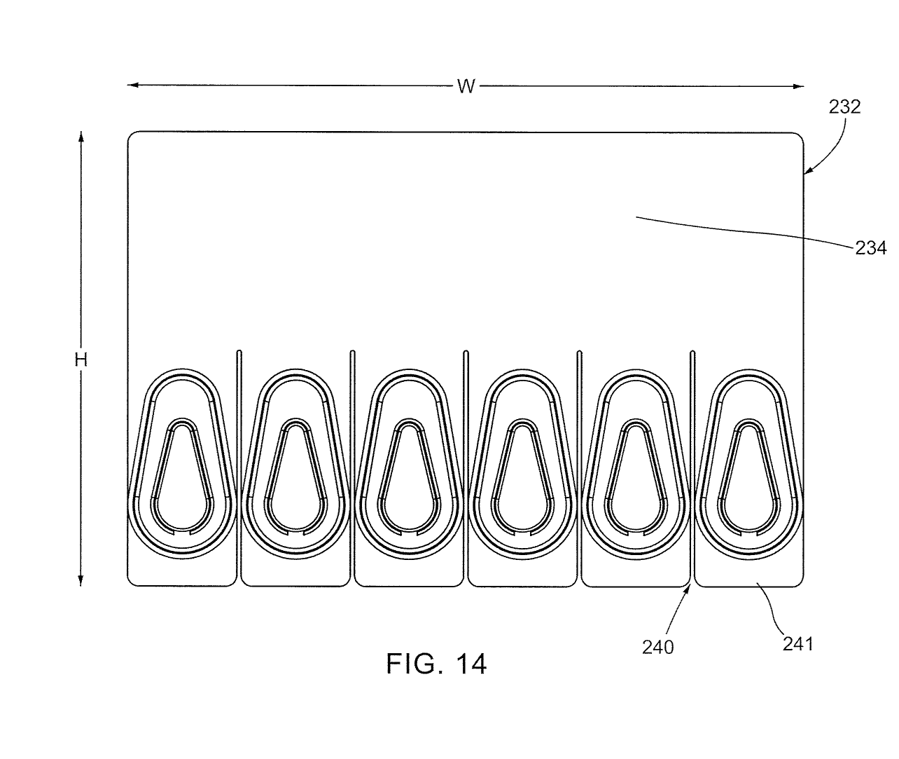

The disposable cartridge 104 is also configured to have as little thermal mass and thermal resistance as possible to further increase thermal cycling rates. For example, the thickness of the portion of the cartridge between the thermal cycler and the sample volume is minimized. Additionally, the material(s) from which cartridge 104 is formed is selected to have a high thermal conductivity. Furthermore, to reduce the thermal contact resistance between the thermal cycler and the disposable cartridge (which is inherent at the interface of two solid surfaces), the disposable cartridge is configured to be mechanically compliant with the thermal cycler. This can be achieved, for example, by forming slits or slots between the reaction wells (see e.g. FIG. 14). This improves the ability of a light clamping force to encourage a consistent and acceptable thermal interface between the disposable cartridge and the thermal cycler.

The wells of the disposable cartridge 104 are arranged linearly. This allows for a simple implementation of optical scanning of the wells, in that the optics of the optical scanning device 124 can be moved by one motion stage, i.e. along a single axis. In some embodiments, however, it may be desirable to configure the optics to move in more than one axis. For example, the carriage and optics can translate in one axis linearly across the sample wells and in a second axis in an up and down direction in relation to the sample wells. Such a configuration would further increase the number of measurements that can be taken within a sample well. Additionally, the optics can be aligned with the widest part of the reaction well to increase the number of data points that can be acquired in order to assess the existence of DNA within the reaction well. This layout also simplifies the thermal cycler assembly, in that a single off-the-shelf thermal electric device can be used. When using multiple devices, the thickness and alignments may become critical to ensure proper thermal operation.

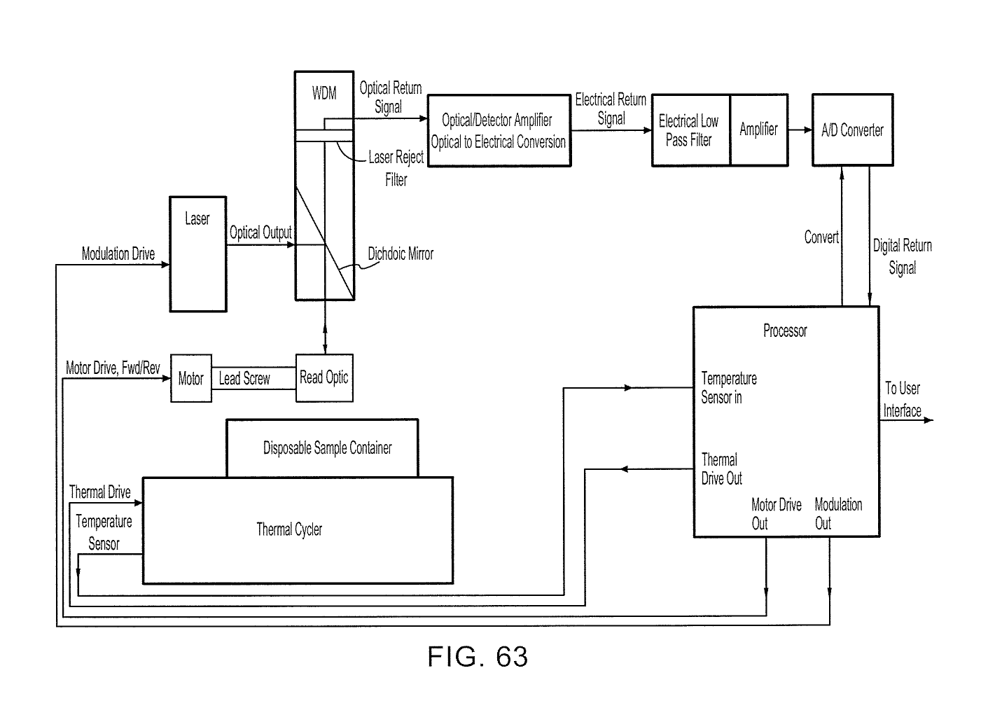

A schematic systems diagram illustrating the operative connections between the various components of a PCR device as described herein is also shown in FIG. 63. FIG. 63 also illustrates an optical path and a thermal path that can be associated with a PCR system as described herein. For example, an optical path is shown between a laser light source, a read optic (e.g., a fiber focuser), a disposable cartridge, a detector and a processor. A thermal path is shown between the processor, a thermal cycler and the disposable cartridge.

In use, the detection of PCR signals is performed by illuminating a sample with photons (e.g., with the light source in the optical scanning device 124) to activate the fluorophores in the sample, and detecting photons released by the fluorescent response of the fluorophores. Specifically, the reaction wells in the cartridge 104 are filled with a test sample and PCR solution. The cartridge 104 is placed within the PCR device 102 adjacent the thermal cycler 122. When the device is activated (e.g., a power switch is turned on), the thermal cycling starts and the PCR reaction begins. The optical scanning device 124 monitors the fluorescent signals in each well and the control device 114 records the signal intensity. The results are analyzed and displayed on the user interface screen 108 and/or on a central monitoring station 112 (see e.g., FIG. 2). The PCR system 100 described herein can perform this detection in several different ways.

For example, a first method illuminates the sample for an extended duration and then takes a single measurement of the returned photons in a small region of the sample. One disadvantage of this method is the possibility of photobleaching the sample (discussed in more detail below), which would result in a diminishing number of returned photons and a smaller detected signal measurement. Another disadvantage is the possible use of less than the optimal laser power for maximum sensitivity while avoiding photobleaching.

A second method, referred to as The Pulsed Small Optic method without Hunting is similar to the above method, except that the laser is modulated by being pulsed on only long enough to measure the returned photons. The laser may be pulsed many times and the measurement taken many times, to allow averaging or other noise reduction methods to be applied to the detected return photon signal. This method reduces the laser exposure of the sample and the subsequent possibility of photobleaching. This method takes advantage of the sample mixing that occurs inside each sample during each thermal cycling step. This mixing causes the photon emitters in the sample to be nominally uniformly distributed throughout the sample volume, which allows measurement from a small region of the sample and the use of an optic that is small compared to the size of the sample.

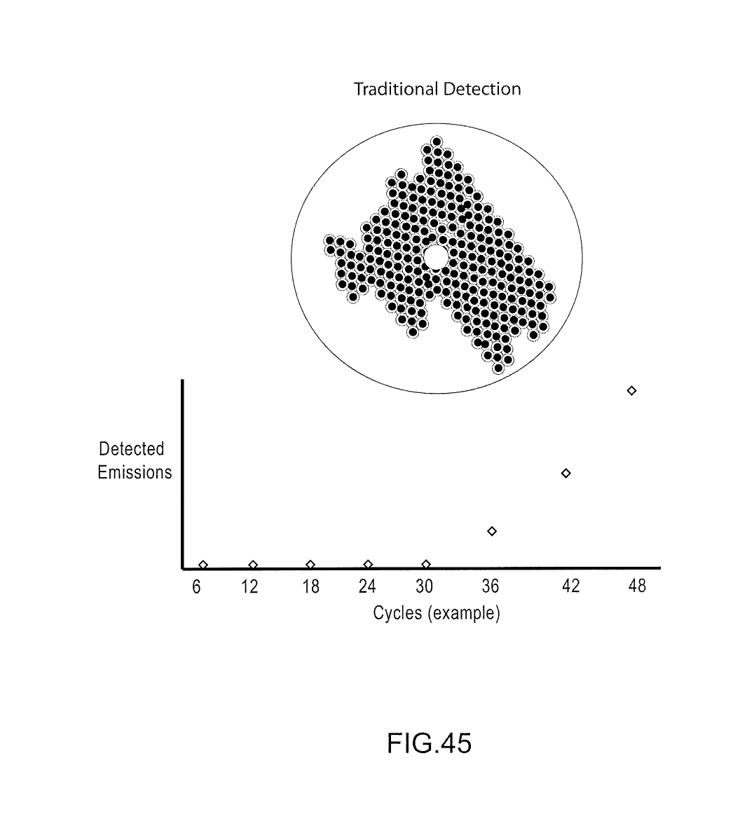

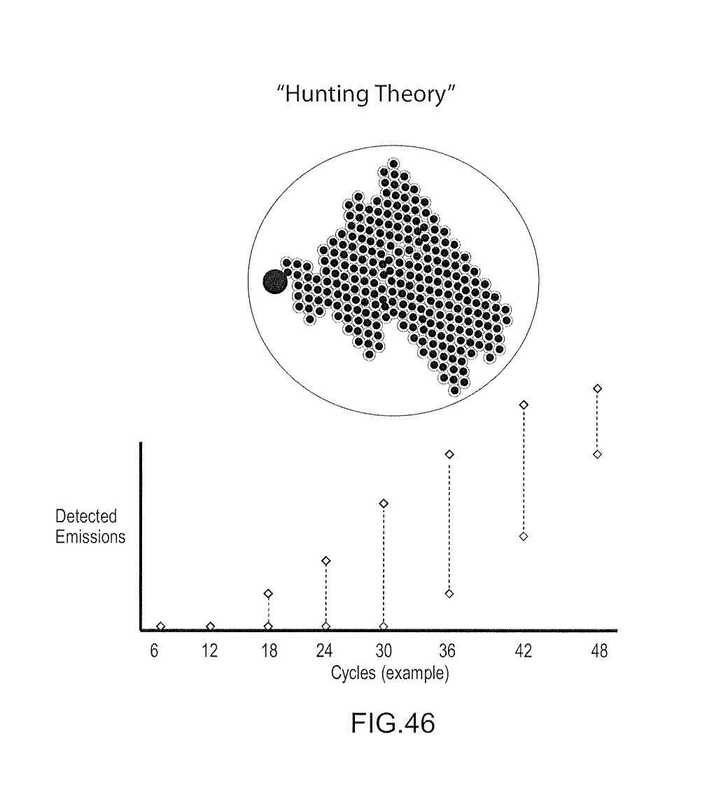

A third method, referred to as The Pulsed Small Optic method with Hunting is similar to the Small Optic method above, except that measurement of the returned photons in the sample is made at many different locations within the sample. The term “Hunting” as used herein refers to the activity of dynamically searching for DNA to enable earlier detection of amplification. This method can be used, for example, with samples in which uniform mixing does not occur during the thermal cycling, and the sample must be searched, or hunted, for the returned photon signal. This may be implemented, for example, by mechanically scanning the optics across a sample with a linear or other motion (as described above). In this method too, the laser may be pulsed many times and the measurement taken many times at each location within the sample, to allow averaging or other noise reduction methods to be applied to the detected return photon signal. FIG. 45 is a graph and illustration showing the detected emissions using a traditional detection method, and FIG. 46 is a graph and illustration showing the detected emissions using a Hunting method.

The Pulse methods of operation has several advantages over the first method described above, in that it allows the PCR return photon signal to be increased while reducing photobleaching of the sample. An example calculation of maximum laser power to maximize sensitivity while avoiding photobleaching is described below.

A fluorophore will emit a lower energy photon in response to being stimulated by a higher energy photon for some lifetime, e.g. 100,000 or more stimulations/emissions. After this lifetime, the fluorophore is said to be photobleached and is permanently damaged and will no longer emit photons. In one example, the fluorophore is excited by photons within a range of wavelengths centered around 645 nm. The laser is therefore selected to emit photons nominally at 645 nm, with each photon containing about 3×10⁻¹⁹ Joules of energy. The nominal area of the reader optics projected on the sample is, for a focus spot of its 2.2 mm in diameter, 3×10⁻⁸ m². If the laser power is 1 mw=1 mJ/sec, then the number of photons emitted per second is 3.3×10¹⁵ photons/second. When illuminated, a sample emits photons which are collected by the optics and sent to the detector. The detector may have an amplifier with a gain to the output of 1.1×10⁸ volts per watt of optical input power. This output voltage may be electrically filtered to remove high frequency noise, and then amplified again with a gain=0.3 to accommodate the input voltage range of the A/D converter. It should be understood that the gain can vary depending on the particular optical devices. It may be desirable, for example, to set the gain as high as possible, while still preventing noise from usurping the A/D voltage range. For B-type fluorophore, the signal at the A/D converter is about 2 volts. Given the conversion gains, the optical input power from the sample is 2/3.3×10⁸=6×10⁻⁸ watts=6×10⁻⁸ Joules/second. This represents 6×10⁻⁸/3×10⁻¹⁹=2×10¹¹ photons/sec emission rate from the sample B-type material. With a laser pulse time of 1 ms, then 2×10¹¹/1×10³=2×10⁸ photons were emitted from the sample. If there are 1×10¹³ B-type fluorophores in the sample, then each would emit 2×10⁸/1×10⁵=2×10³=2,000 photons. When this laser illumination is repeated for up to 50 cycles, the total fluorophore exposure would be (50 cycles)×(2000 photons/cycle)=100,000 photons. This would be below the exposure needed for photobleaching to occur. If the fluorophore efficiency is 0.33, such as for Alexa 647, then 3 excitation (laser) photons are required for each emitted photon, or 300,000 photons of exposure, and a re-calculation of the maximum laser power that could be used given the lifetime of the Alexa 647 fluorophore. In addition, the sample mixing caused by the thermal cycling would allow different fluorophores to be exposed to the laser, rather than the same fluorophores over and over. This would reduce further the possibility of photobleaching and allow higher laser “interrogation” power. The volume of the sample is about 0.012 ml or 1.2×10⁻⁸ m³. There are estimated to be 1×10¹³ fluorophores in this volume. This is 1.2×10⁻⁸/1×10¹³=1.2×10⁻²¹ m³ per fluorophore.

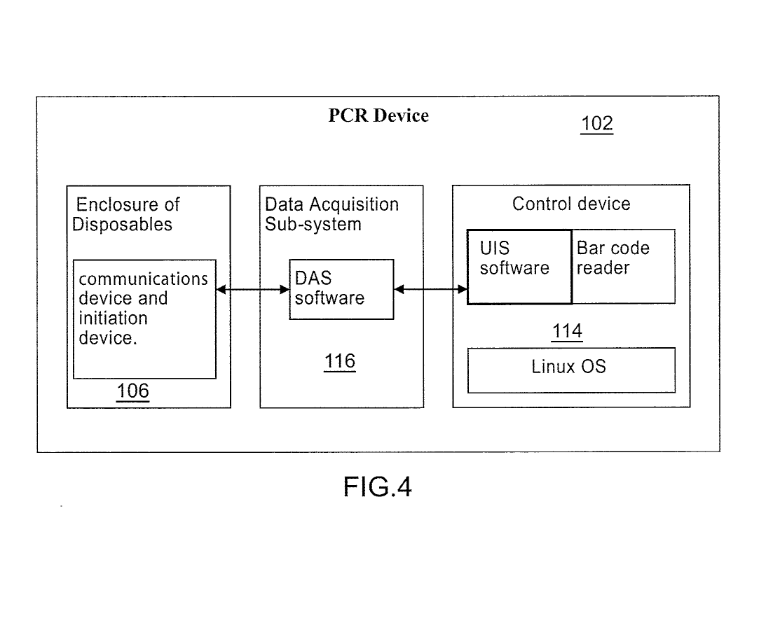

As mentioned above, the PCR device 102 can also include a control device 114 and a data acquisition sub-system 116, as shown in FIG. 4. FIG. 4 is a schematic illustration of a portion of the PCR device 102 illustrating the inter-relationship of the control device 114, data acquisition sub-system 116 and the disposable cartridge enclosure 106. The control device 114 provides the user interface and control functions required to set-up the PCR system 100 for monitoring, for example, a pathogen sample test. The control device 114 includes User Interface Software (UIS) that implements this functionality on the PCR device 102.

The PCR device 102 can include sensors that can take sample test measurements and pass the information to the control device 114. The User Interface Software (UIS) runs on the control device 114, manages the user interface and interfaces to a barcode reader. The bar code reader manages the input of the information from the bar code 118 on a disposable sample holder 104 (e.g. insertable cartridge) to the PCR device 102. The bar code information can be used to uniquely identify a disposable cartridge 104 and associate it with a particular test. In addition, in some embodiments, the bar code reader can be programmed to read a user identification (ID) badge. The data acquisition sub-system 116 includes DAS software that can determine whether or not the disposable cartridge 104 is present in the PCR device 102, and pass that information to the UIS.

The status of whether the disposable cartridge enclosure 106 is open or closed can also be detected by the DAS and passed to the UIS running on the control device 114. The UIS can also open or close the disposable cartridge enclosure 106 by sending a command to the DAS, and the DAS can fulfill the command.

The DAS software can be hosted, for example, on a custom circuit board based on the Atmel 8/16-bit Xmega Microcontroller running firmware developed in the C language. Other circuitry can be included to level shift, power, self test, and otherwise monitor the system sensors and the processor. Captured sensor data will be stored within RAM (not flash) to assure integrity; specific managed and pretested RAM locations will be used for easy assembly of the framework system defined above. A previously sent framework will be saved by the sensor board until after acknowledgement.

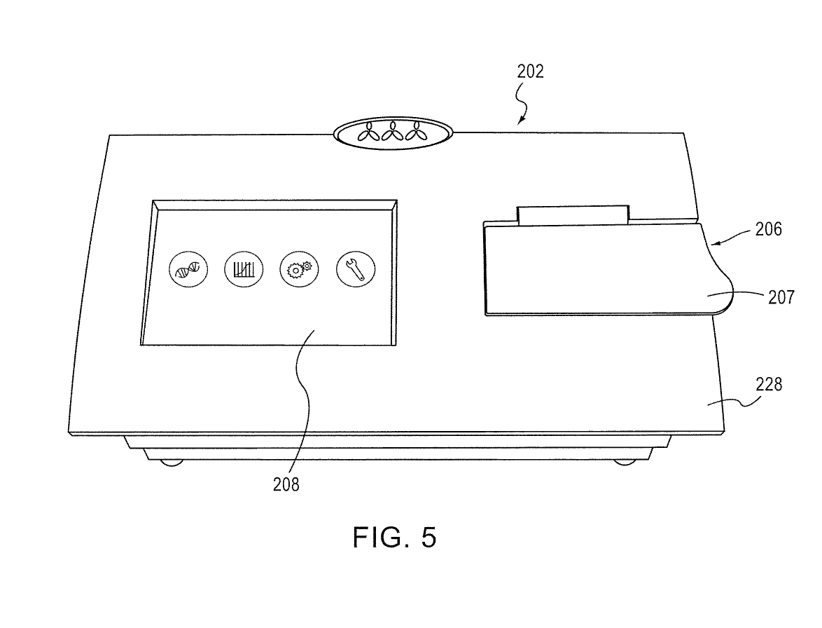

FIGS. 5-9 illustrate an embodiment of a PCR system 200 that includes a PCR device 202 configured to receive a disposable sample holder 204. The PCR device 202 can be sized and configured to be self-contained and weigh about 15 pounds. The PCR device 202 can be sized for example, with a width of approximately 15 inches (381 mm), a depth or length of approximately 12 inches (305 mm), and a height of approximately 10 inches (254 mm). In some embodiments, the PCR device 102 can be sized, for example, with a width of approximately 16 inches (406 mm), a depth or length of approximately 9.5 inches (241 mm), and a height of approximately 7 inches (178 mm). Various components of the PCR device 202 can be formed, for example, by injection molding and robotically assembled. Although not necessarily described in detail with reference to this embodiment, the PCR device 202 can include any of the same features, and function in the same or similar manner as described herein for other embodiments. For example, the PCR device 202 can include a control device, a connection port for interface with a network, supporting software and/or hardware, power switch, printer capability, etc.

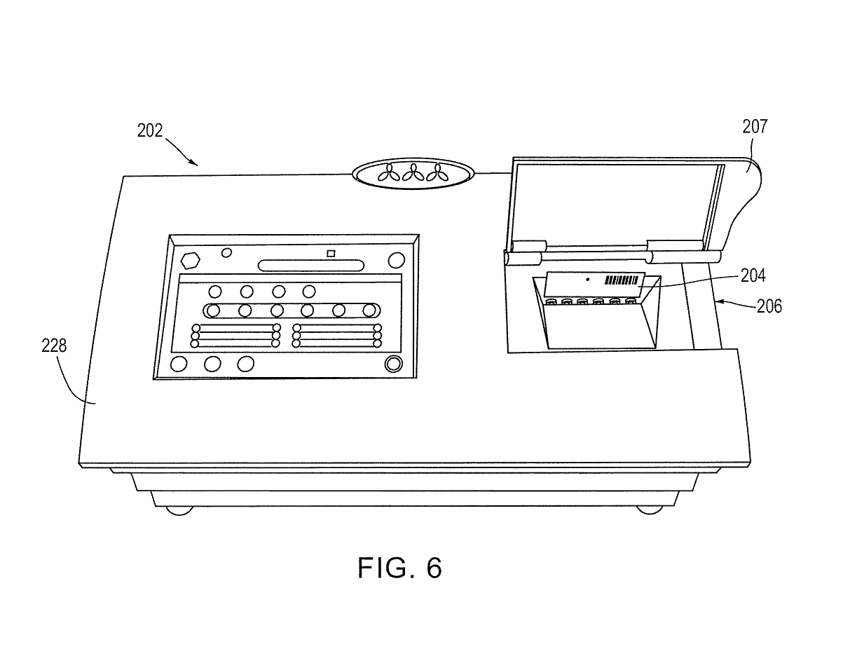

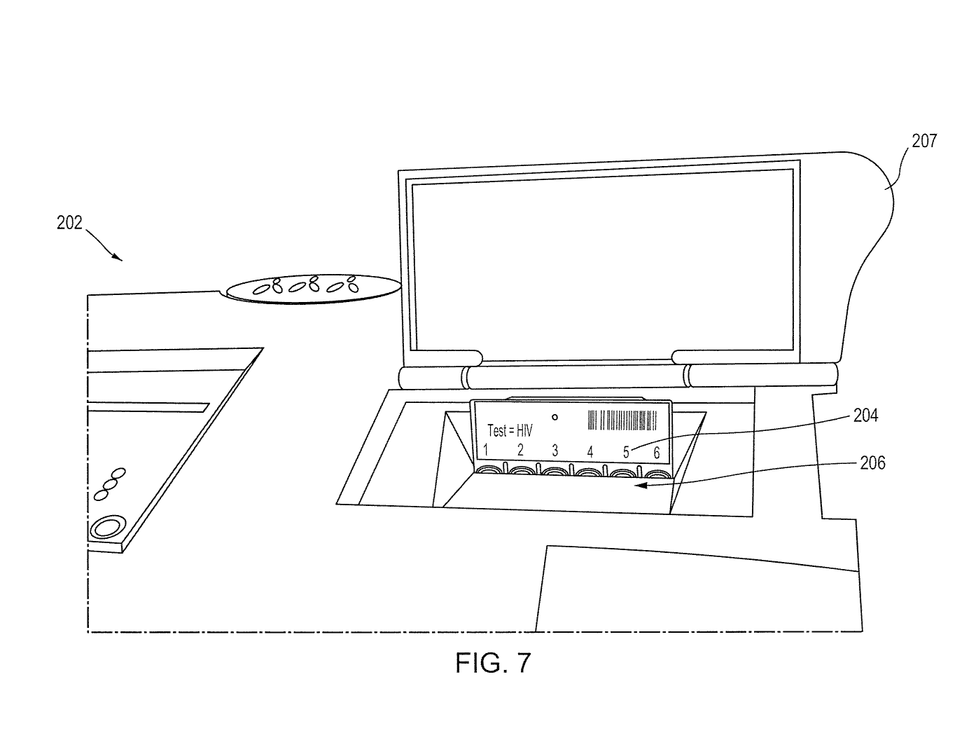

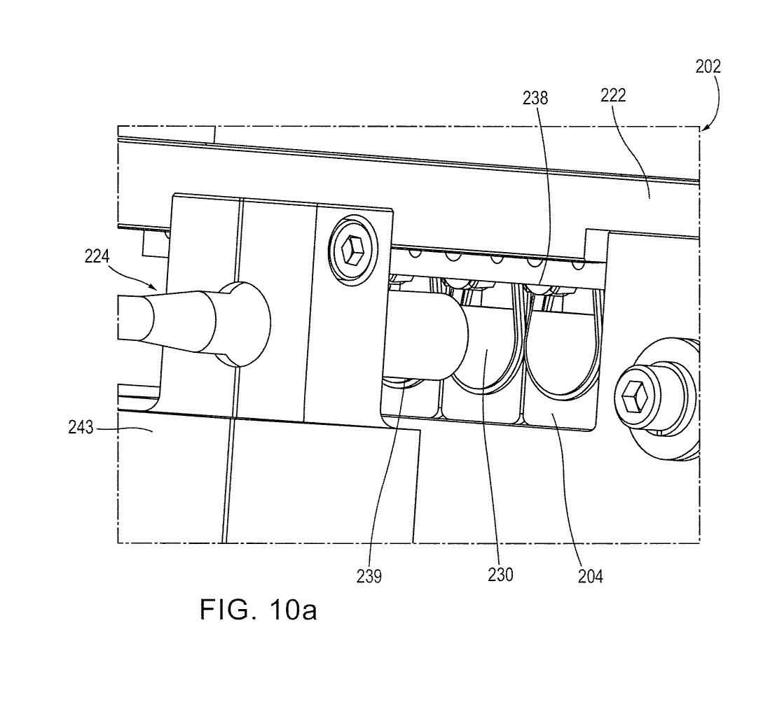

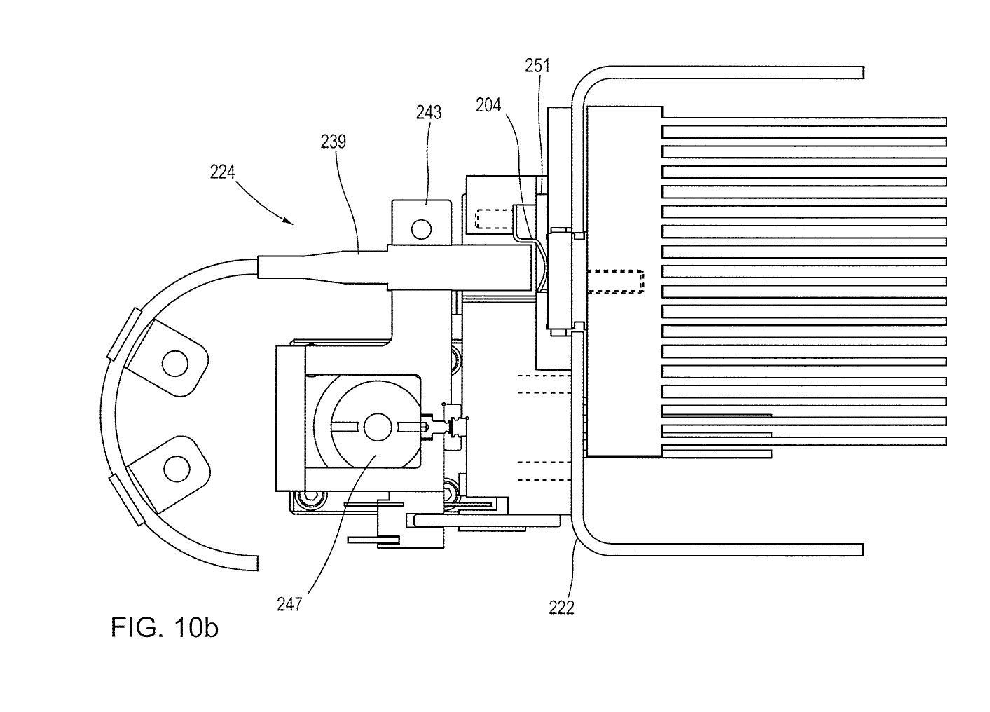

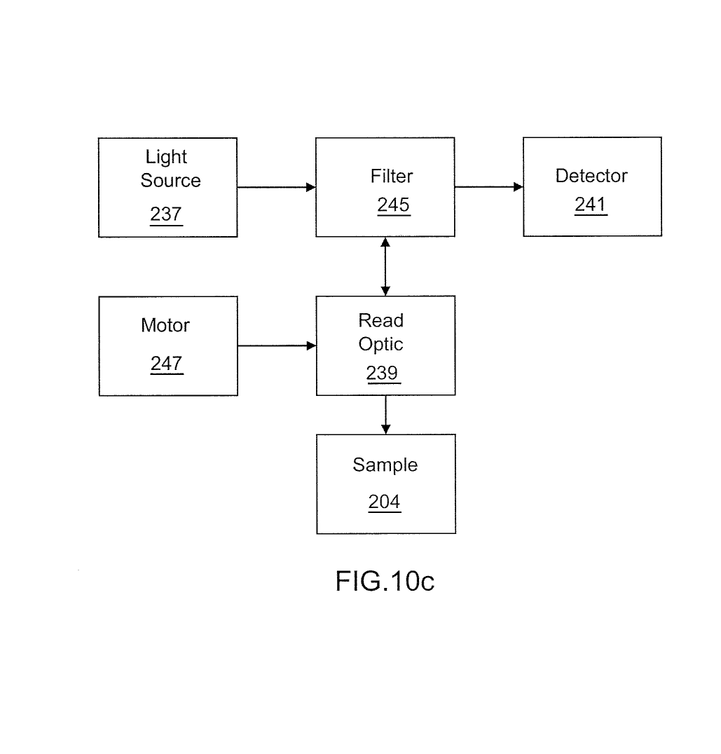

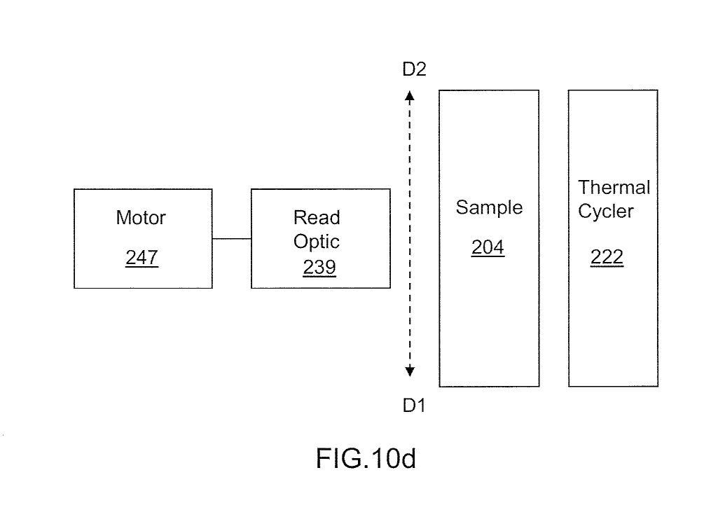

As shown in FIGS. 5-7, the PCR device 202 includes a housing 228, a touch screen user interface 208, and a cartridge receiving portion 206 (see FIGS. 6 and 7). A door 207 is coupled to the housing 228 to provide access to the cartridge receiving portion 206. The door 207, can be, for example, hingedly coupled to the housing 228. The PCR device 202 also includes an optical scanning device 224 and a thermal cycler 222 (shown in FIGS. 10a and 10b).

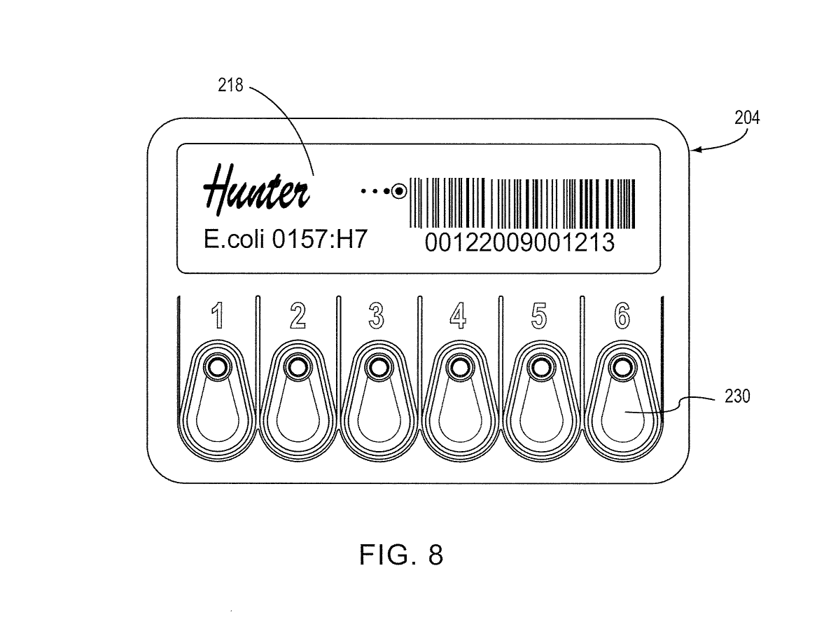

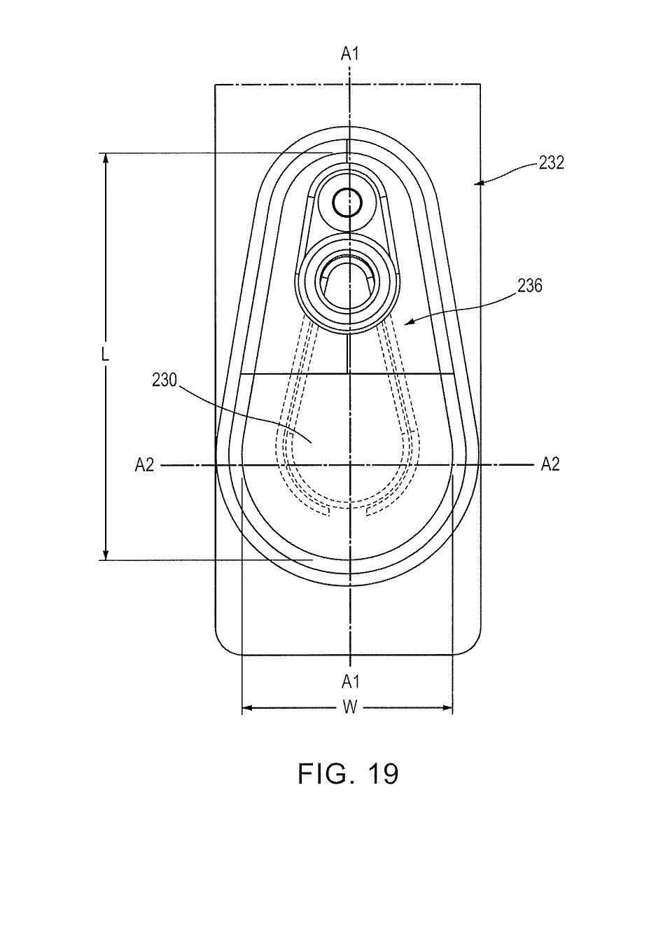

FIG. 8 is a front view of a portion of a disposable cartridge 204. The disposable cartridge 204 includes a bar code 218 and multiple reactor wells 230 (in this embodiment, six wells). As discussed above, the reactor wells 230 are disposed in a linear arrangement and the cartridge can be inserted into the PCR device 202 in a vertical orientation, as shown in FIGS. 6 and 7. The linear arrangement of the wells 230 and the vertical orientation of the cartridge 204 allows the optical scanning device 224 (shown in FIGS. 10a and 10b) to move across the wide portion of the reactor wells 230 in one direction. Also as discussed above, with the disposable cartridge 204 disposed in a vertical orientation, the optical scanning device 224 can translate along an axis substantially perpendicular to an axis A1 of the reaction wells 230 that is parallel to a length L of the reaction wells (shown in FIG. 19).

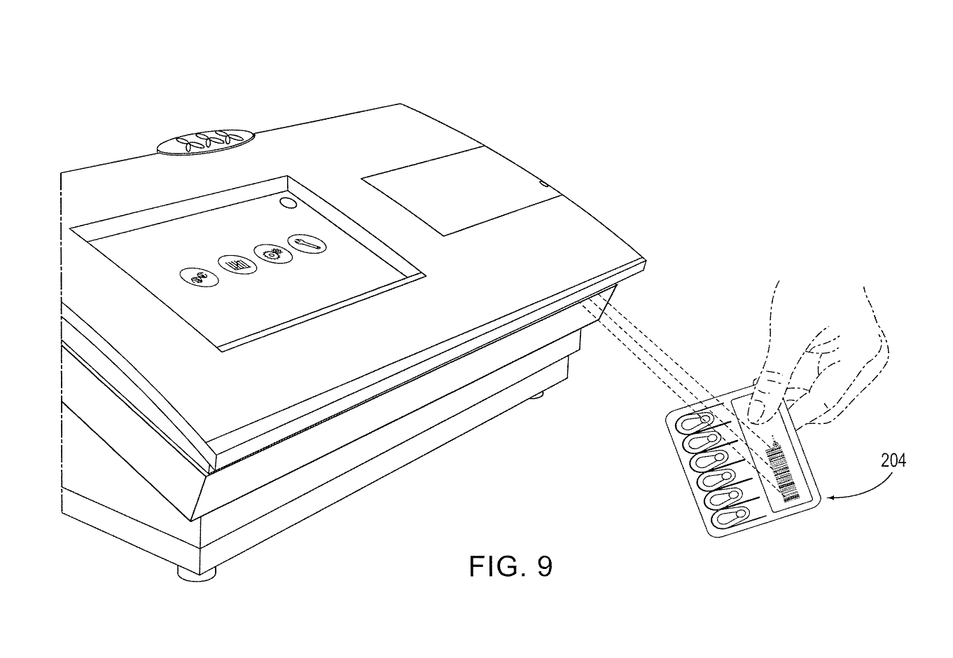

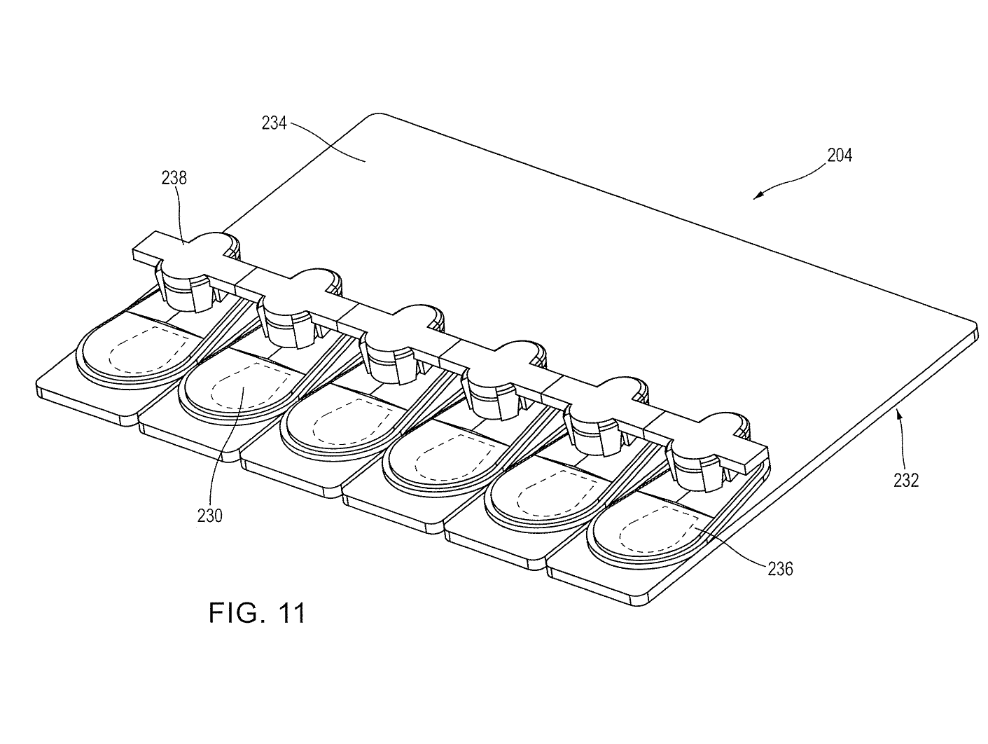

Prior to placing the cartridge 204 in the PCR device 202, the bar code 218 of the cartridge 204 can be “scanned” (see e.g., FIG. 9), e.g., read by a bar code reader (not shown in FIGS. 5-9). As described above, the thermal cycler 222 can provide a clamping force over the cartridge cap 238 (shown e.g. in FIG. 11) to help further seal the cartridge 204.

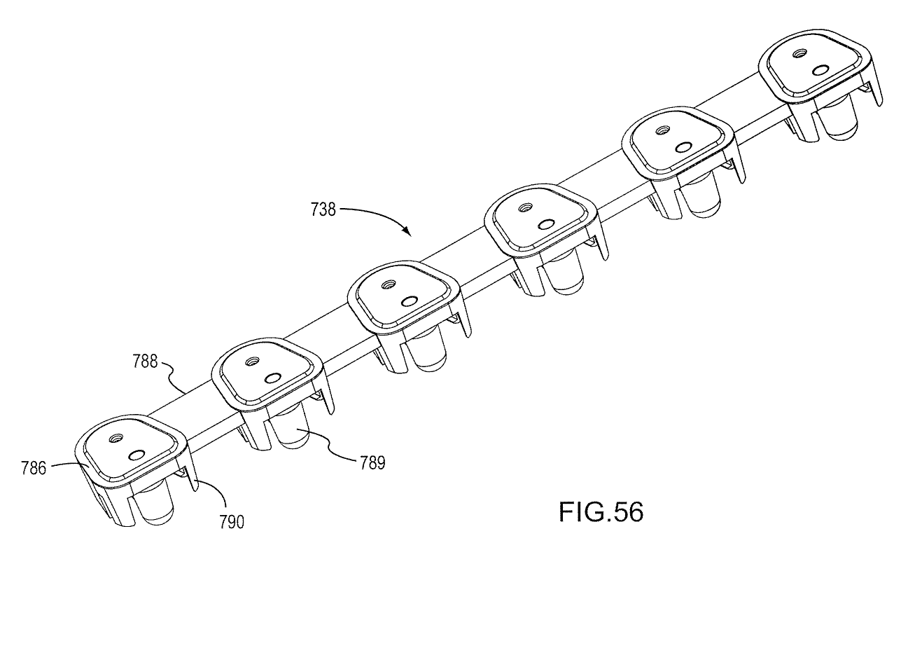

The optical scanning device 224 includes a light source 237, which can be, for example, a laser (shown FIG. 10c, which is a schematic illustration of the optical path of optical scanning device 224) (see also the schematic illustration of FIG. 63, which illustrates examples of an optical path and a thermal path associated with a PCR system, such as PCR system 200). The light source 237 can have, for example, a peak wavelength at 635 nm (+/−10 nm) and an output power of between 4 mW and 25 mW. The optics of the optical scanning device 224 can include, for example, a read optic 239, such as, for example, a pigtailed fiber focuser with, for example, a working distance of 2.2 mm. The optics in the optical scanning device 224 can provide an optical path between the light source 237, the sample (e.g., disposable cartridge 204), and a detector 241 and the samples in the disposable cartridge 204. The read optic 239 can be mounted to a carriage 243 (see FIGS. 10a and 10b) of the optical scanning device 224 as previously described. The carriage 243 can have, for example, a nominal position resolution of 0.05 mm/step; have a means to detect a near travel limit; have a means to detect a far travel limit and have a means to detect well start position. A motor 247 is operatively coupled to the carriage 243 to move the carriage 243 back and forth in front of the disposable cartridge 204 in a direction D1 and a direction D2, shown in the top view schematic of FIG. 10d. The detector 241 of the optical scanning device 224 can, for example, be capable of light detection at 0.1 nW minimum and at 50 nW maximum. The detector 241 can have a bandpass filter 245, such as, a wavelength division multiplexer (WDM) (see also FIG. 56). The filter 245 can be optimized, for example, at 635 nm and 660 nm wavelength; be mounted such that a port leads to the light source 237; be mounted such that a port leads to the detector 241; be mounted such that a port leads to the read optic 239; and can have a bandpass filter at the port leading to the detector 241. The bandpass filter 245 can be optimized, for example, to reject the laser wavelength on the return pass, and pass only the PCR wavelength (e.g., the light of interest). It should be understood that the operating parameters provided above for various components are merely examples. In addition, although one laser light source is shown and described, the optical scanning device 224 can be configured to accept multiple lasers at different wavelengths that can be coupled to the read optic 239.

The thermal cycler 222 includes a heat plate 251 as shown, for example, in FIG. 10b. The interface between the heat plate 251 and the disposable cartridge 204 (as described in more detail below) allows for the heat plate to be much smaller in size and therefore, lower in mass, as compared to other PCR systems available in the art. For example, the overall weight of the heat plate 251 can be, for example, about 40 grams. The heat plate 251 can have a width, for example, of 16.5 mm, a length, for example, of about 78 mm, and a depth, for example, of about 4 mm.

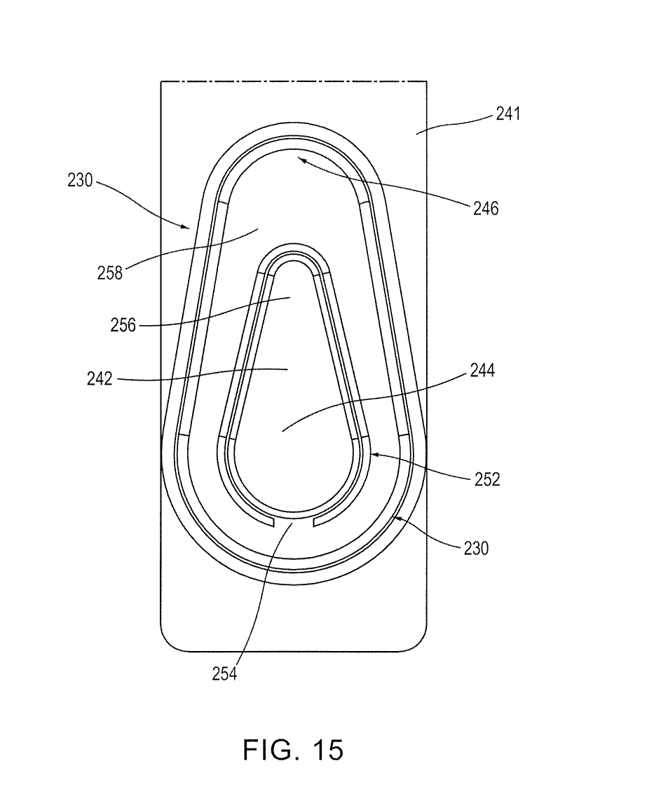

FIGS. 11-24 illustrate various details and features of the disposable cartridge 204. It should be understood that other disposable cartridges described herein (e.g., cartridge 104) can include the same or similar features and/or functions. In this embodiment, the cartridge 204 includes a base 232 with multiple reaction wells 230 and a bar code mounting surface 234 (shown without bar code 218). The cartridge 204 includes multiple covers 236 each configured to be coupled to a reaction wells 230, and a cap member 238. The covers 236 can be, for example, ultrasonically bonded to the base 232, adhesively attached or both. The cartridge 204 in this embodiment includes six reactor wells 230, but it should be understood that a cartridge as described herein can include other numbers of reactor wells 230, for example, less than six (e.g., one, two, etc.) or more than six.

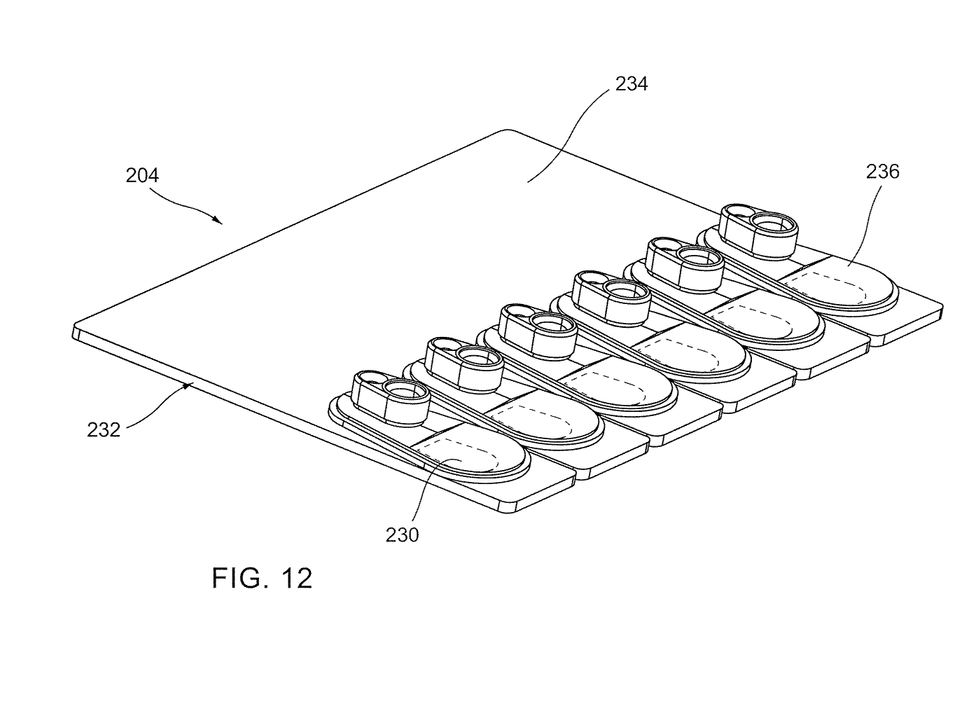

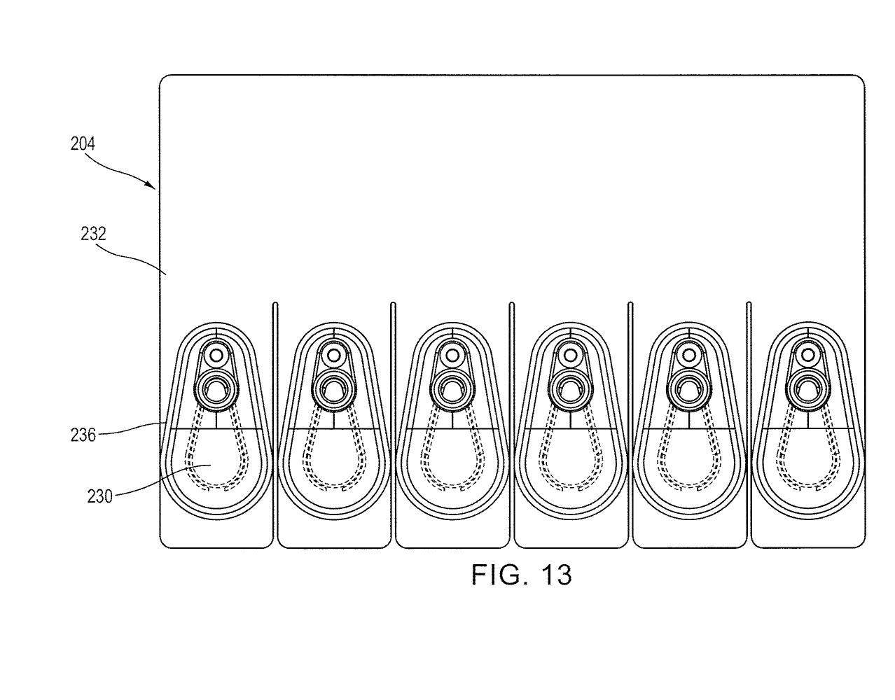

FIGS. 12 and 13 illustrate portions of the cartridge 204, including the base 232 and the covers 236 as assembled (without the cap member 238). FIGS. 14-16 illustrate the base 232. As shown in FIG. 14, the base 232 defines slots 240 between each of the reactor wells 230. The slots 240 provide independent flexible fingers 241 to allow for individual seating of the reactor wells 230 within the PCR device 202. The slots 240 include a radius at a top portion 248 to help prevent fractures during ultrasonic welding (discussed in more detail below) when the covers 236 are secured to the base 232. For example, the top of the slots 240 can have a radius of 0.25 mm. Each reaction well 230 defines an inner cavity portion 242 with a thermal interface wall 244. A top portion 246 of the reaction wells 230 provides a lead-in shape to provide a poke-yoke for insertion of the cover member 236, thus making cover insertion easier. For example, the lead-in area can have a width or outer diameter of, for example 5 mm. A top portion 256 of the inner cavity portion 242 can have a width or outer diameter, for example, of about 2 mm.

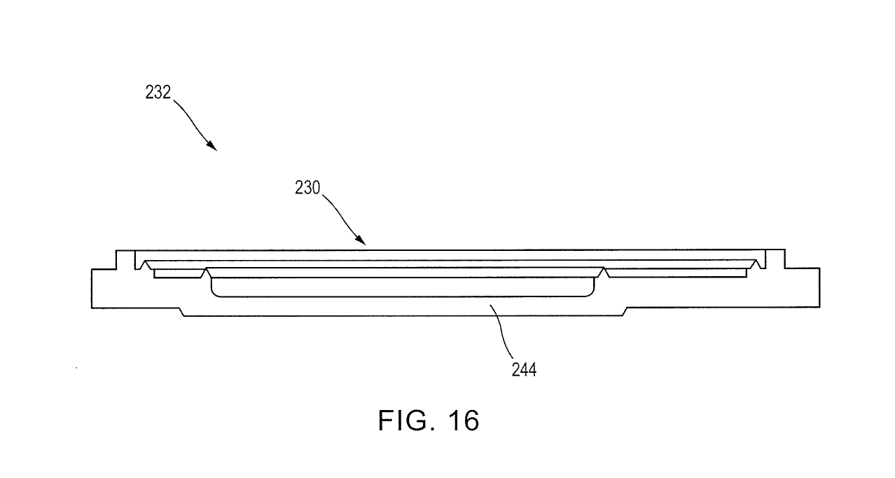

The thermal interface wall 244 is configured to be the thermal interface between the reaction well 230 of the disposable cartridge 204 and the heat plate 251 of the thermal cycler 222. The wall thickness of the thermal interface wall 244 can be, for example, 0.5 mm (see e.g., the side view of FIG. 16). The relatively large cross-sectional area of the inner cavity portion 242 and the relatively thin wall of the interface wall 244 provide for high heat transfer from the thermal cycler 222 to the sample volume. In addition, as discussed above, because of the flat aspect ratio of the cartridge 204, the heat plate 251 can be sized smaller and have a lower mass than in traditional PCR systems.

Each of the reaction wells 230 also includes an inner energy director 250 and an outer energy director 252 used for ultrasonic welding of the covers 236 to the base 232. The outer energy directors 250 can have a height, for example, of 0.25 mm, and provide, for example, a 0.002 mm clearance to the covers 236 (when attached thereto). Various example widths associated with the base 232 are illustrated in the side view of FIG. 16. The inner energy directors 250 define an opening 254 to accommodate a vent tube in the covers 236 (described below). The opening 254 can be, for example, 1.5 mm wide. The bottom surface 258 of the reaction well 230 that is outside of the inner energy director 250 can be used as a bond surface to alternatively or in addition to the ultrasonic welding, adhesively attach the covers 236 to the reaction wells 230. The base 232 can have an overall height H, for example, of 42 mm and an overall width W, for example, of 62 mm, as shown in FIG. 14.

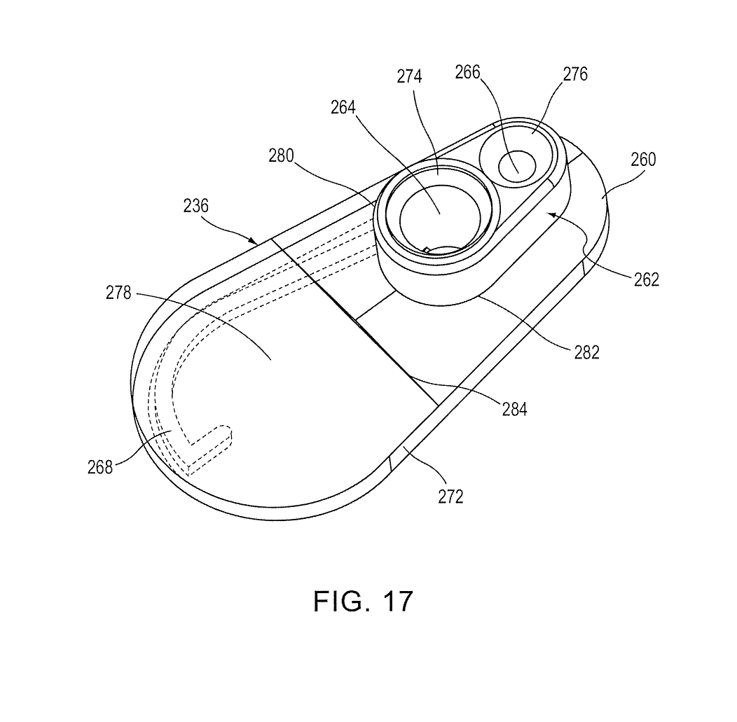

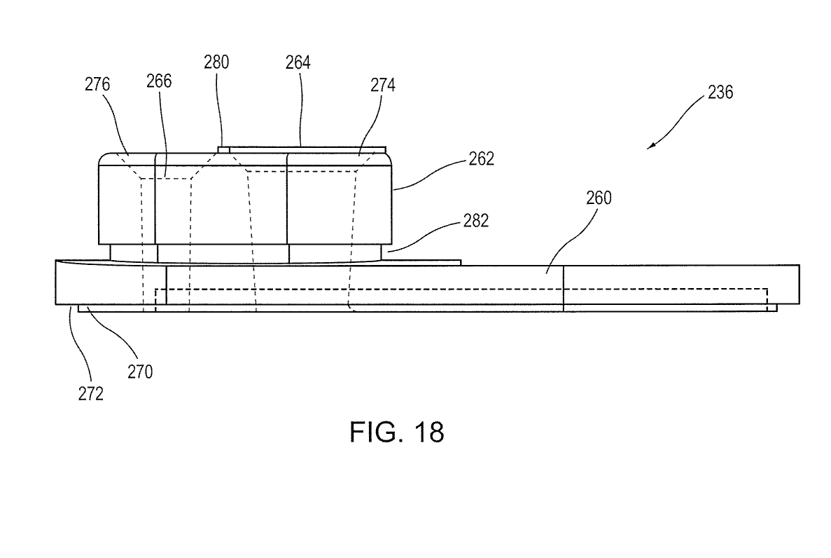

FIGS. 17 and 18 illustrate the cover 236; FIG. 17 is a perspective view and FIG. 18 is a side view. The covers 236 each include a body portion 260 and a raised portion 262. The body portion 260 defines a vent tube 268. The raised portion 262 defines a fill port 264, and an exit port 266 that is in communication with the vent tube 268. The raised portion 362 also defines an angled or chamfered surface 274 surrounding the fill port 264 to allow application of material to more easily identify the fill port 264, and a raised ring 280 for color marking to aid display of the fill port 264. The raised portion 262 also defines an angled or chamfered surface 276 surrounding the exit port 266 for improved sealing to a seal member or portion of the cap member 238 (described below). The vent tube 268 is in fluid communication with the inner cavity portion 242 of the reaction well 230 via the opening 254. The vent tube 268 can have a width, for example, of 0.50 mm.

The covers 236 can each be coupled to the base 232 with, for example, an ultrasonic bond. As shown in FIGS. 12 and 13, a cover 236 is placed over each reaction well 230. A bottom ridge 270 (see side view of FIG. 18) can extend from the body portion 260 such that it is undersized in comparison to the outer energy directors 252 of the base 232 to provide a slight clearance when attached thereto. The body portion 260 also defines a lead-in step 272. The lead-in step 272 can extend outward from the ridge 270, for example, 0.2 mm. The body portion 260 can include a flat, clear wall 278 that is disposed over the reaction well 230 when coupled to the base 332. This allows for optical reading of a test sample within the reaction well 230, and to help maintain the focal depth. A fill line 284 is disposed on the wall 378 and can provide better identification of the filled wells. The raised portion 262 is also shaped to provide a poke-yoke for cap insertion and includes an undercut 280 to provides a snap fit for the cap member 338, as described in more detail below.

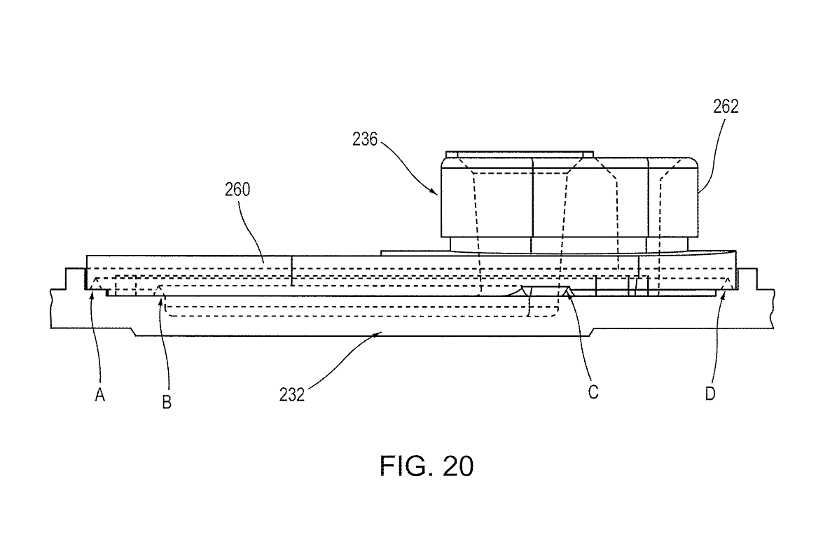

FIGS. 19 and 20 illustrate a portion of the base 232, including a single cover 236 coupled to a single reaction well 230. As shown in FIG. 19, the reaction wells 230 can have a length L and a width W, with the length being greater than the width W. The reaction wells 230 also define an axis A1 extending parallel to the length of the reaction wells and an axis A2 extending parallel to the width of the reaction wells. FIG. 20 also illustrates ultrasonic welding locations A and D on the outside energy director 252, and locations B and C on the inner energy director 250 for ultrasonic welding of the cover 236 to the base 232. These are example ultrasonic welding locations, as other welding locations can be included.

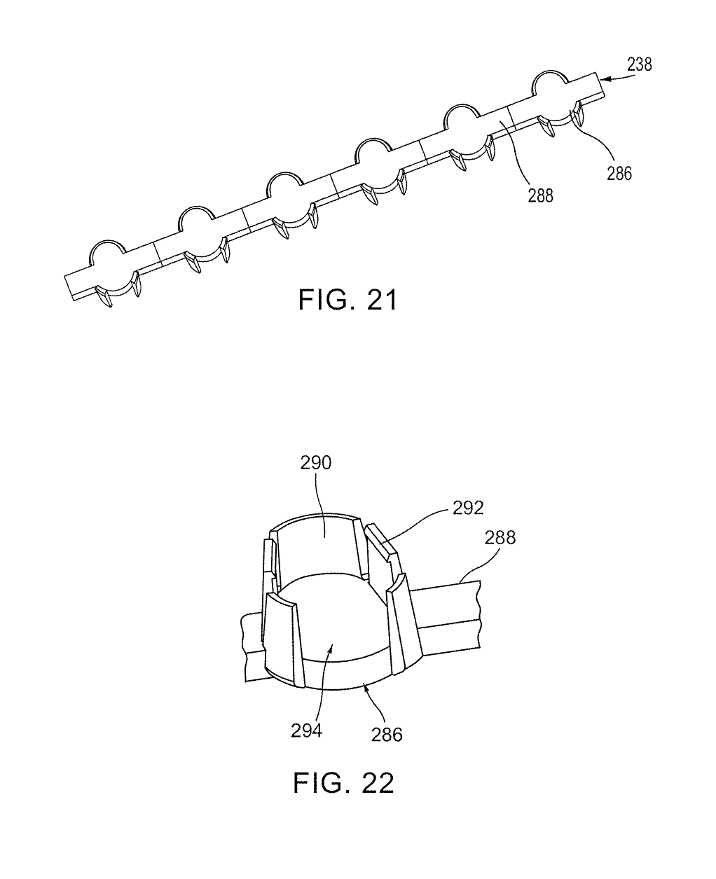

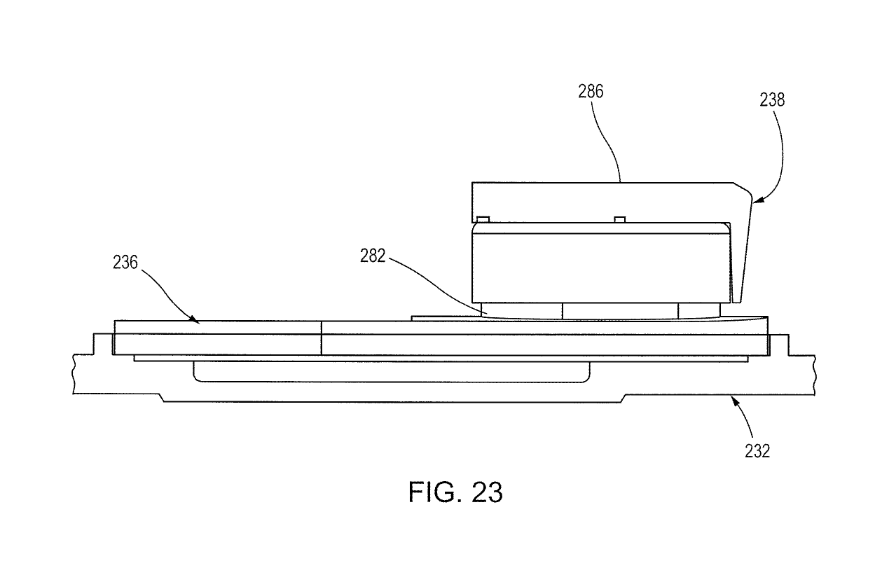

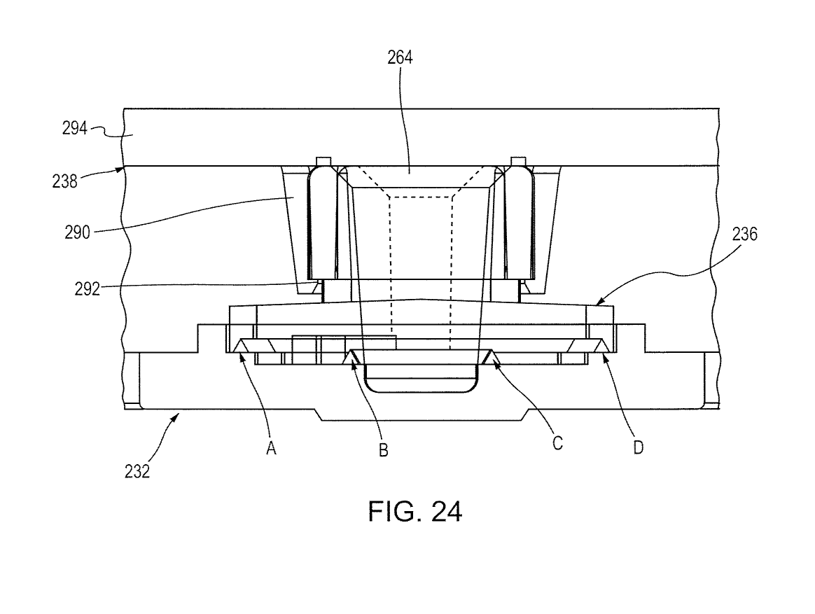

FIGS. 21 and 22 illustrate the cap member 238. The cap member 238 includes multiple caps 286 to matingly couple to the covers 236, and arms 288 disposed between the caps 286. The caps 386 include guides 290 to help align to the fill port 264. Each guide 290 includes a cantilever snap hook 292 configured to snap-fit to the undercut 282 on the covers 236 and secure the cap member 238 to the covers 236. An inner floor 294 of each cap 286 can include an insert molded seal (not shown). For example, a spherical injection molded elastomer structure can be included to improve sealing to the covers 236. FIG. 23 is a side view illustrating a portion of a cap 286 coupled to a cover 236 and the base 232. FIG. 24 is a side view illustrating the snap-fit attachment of the cover to the fill port 264 area of the cover 236. Also shown in FIG. 24 are additional ultrasonic welding locations A and D on the outer energy director 252 and locations B and C on the inner energy director 250.

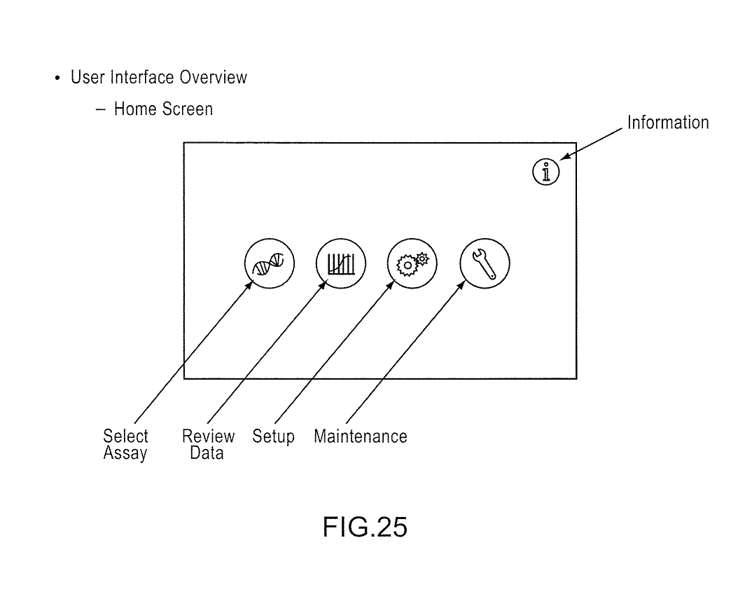

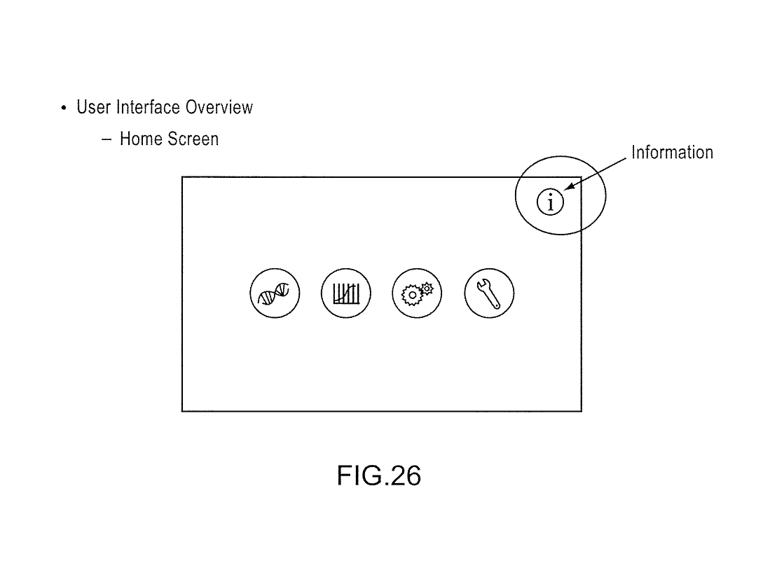

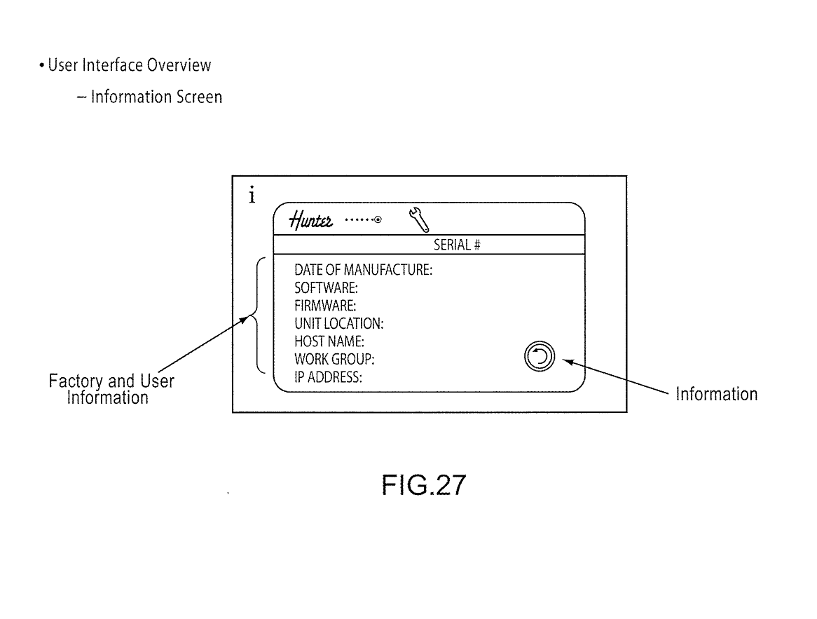

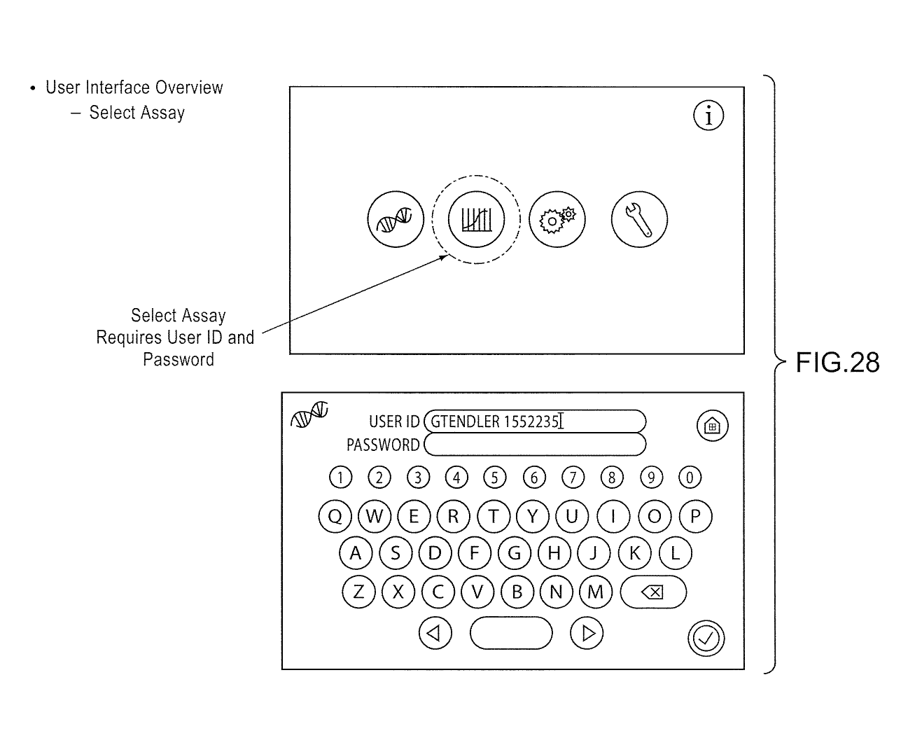

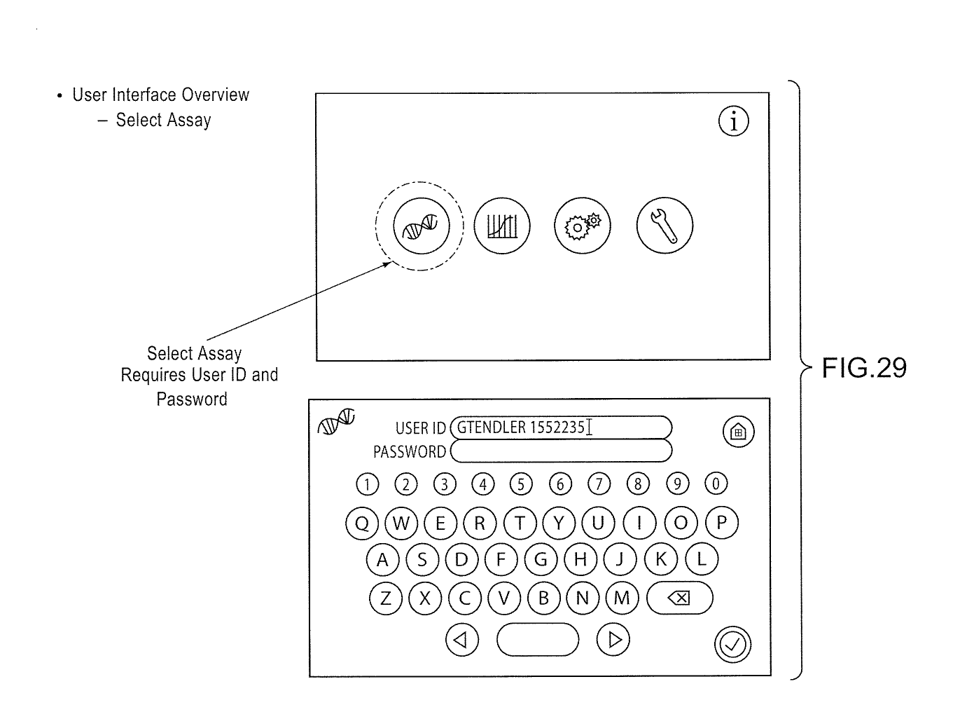

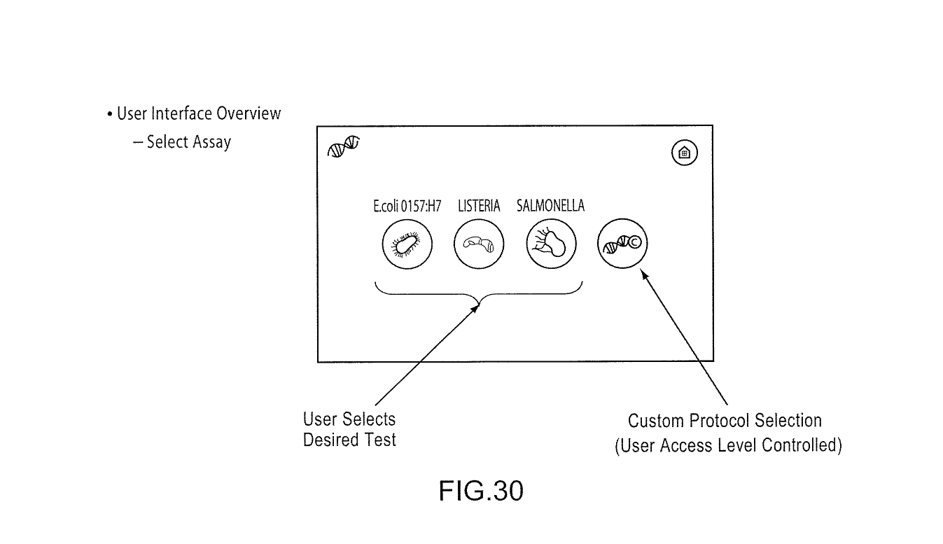

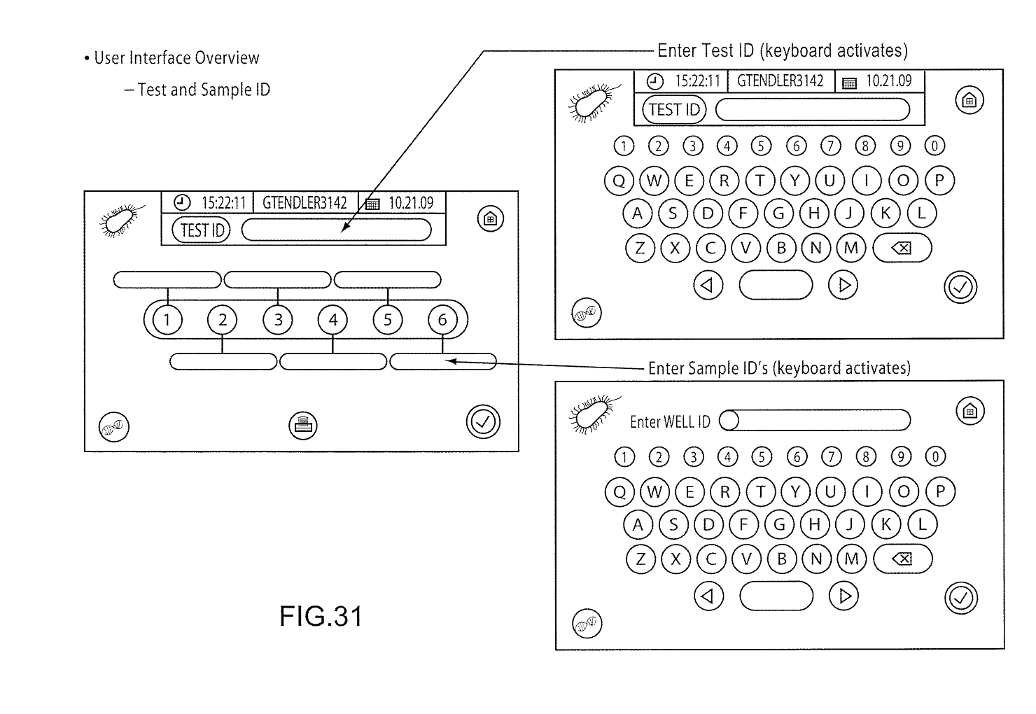

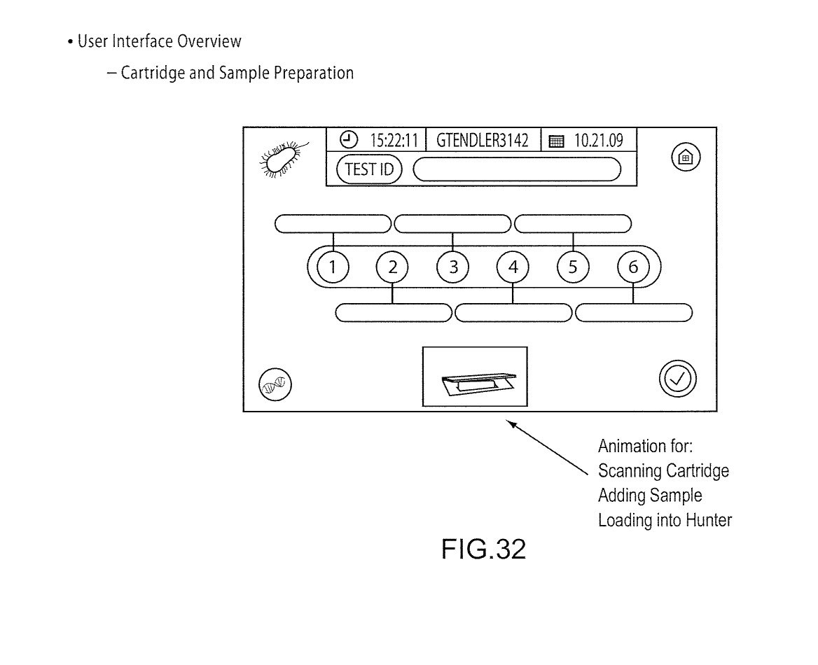

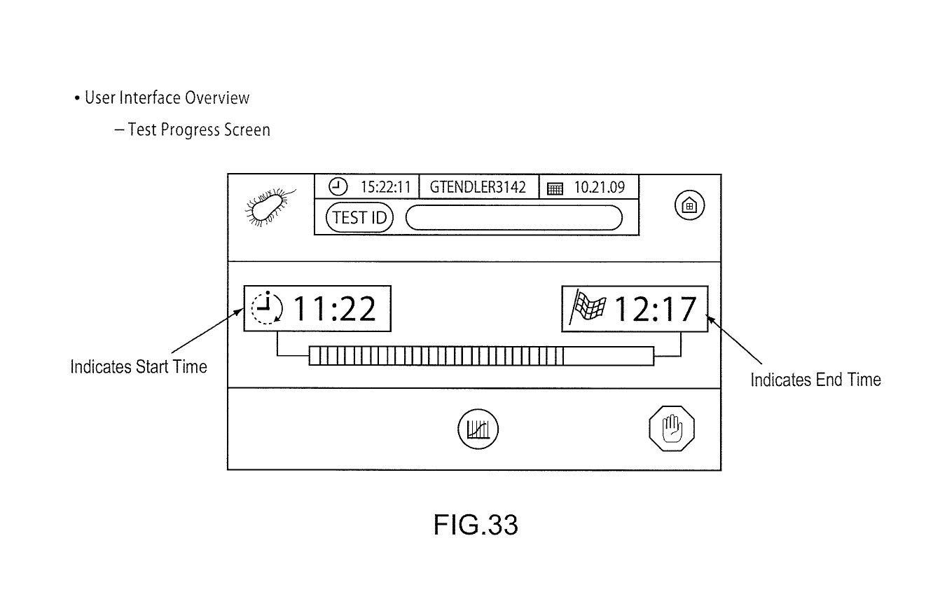

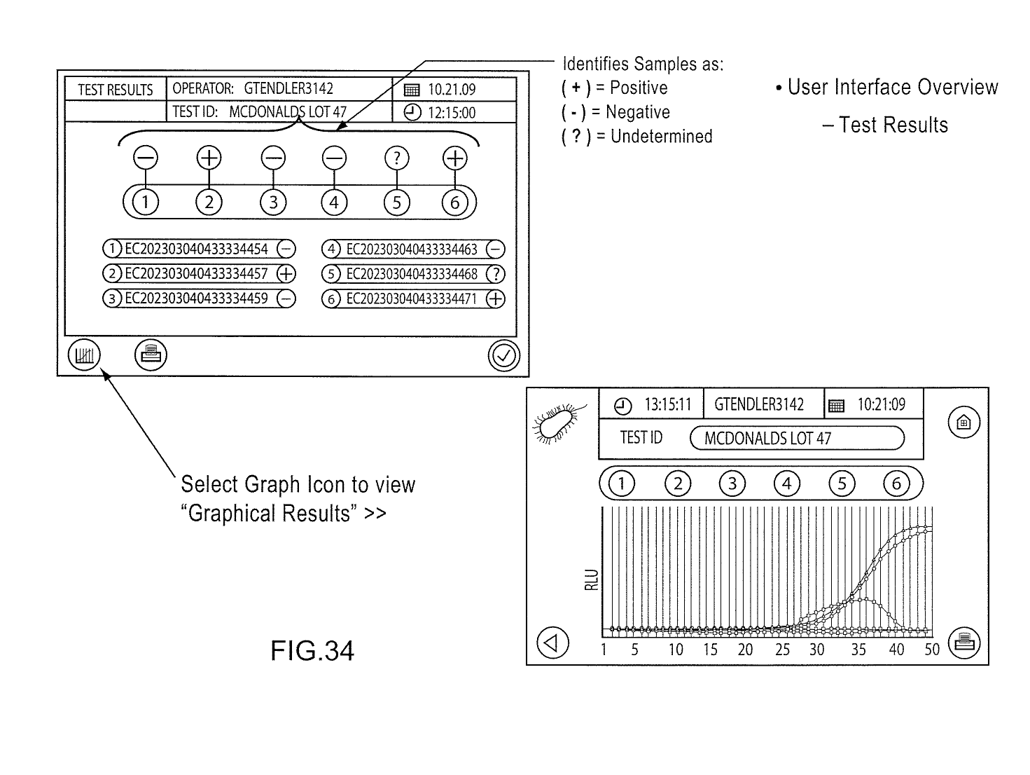

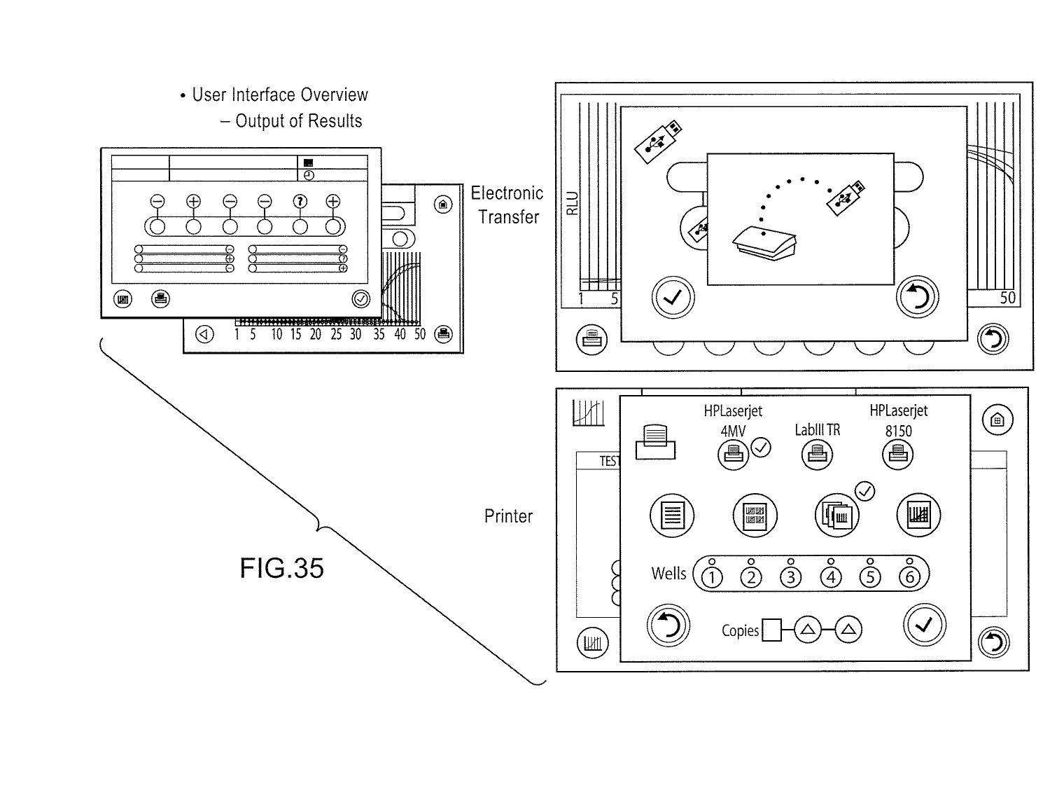

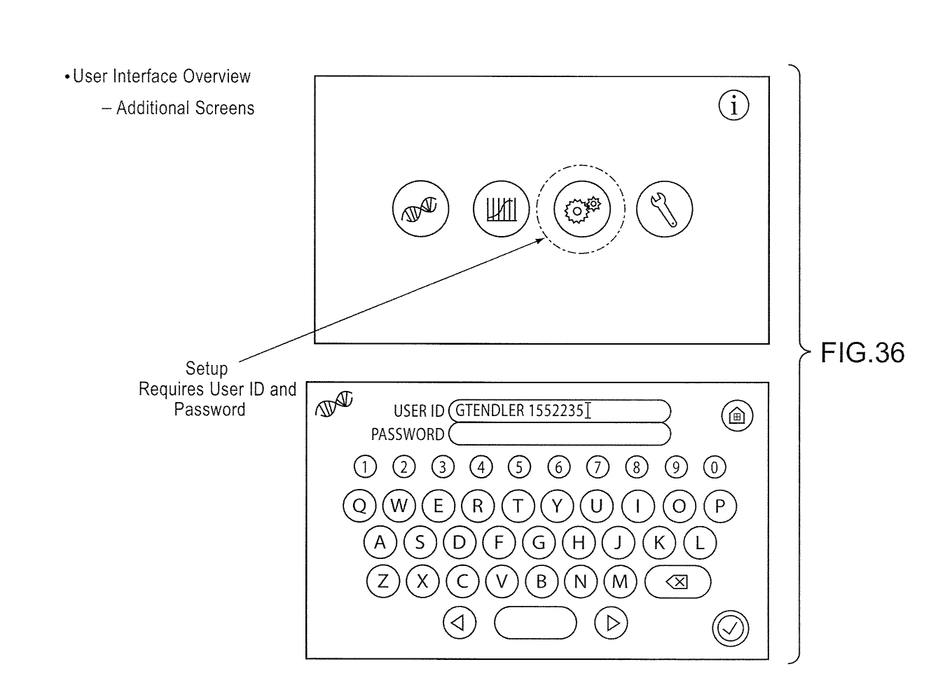

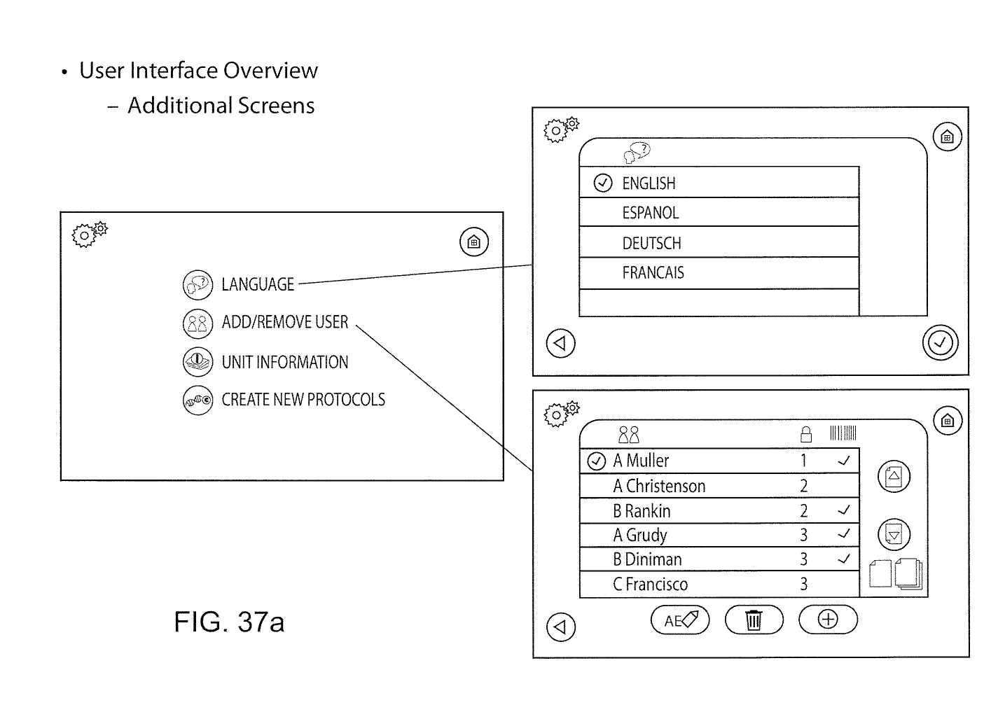

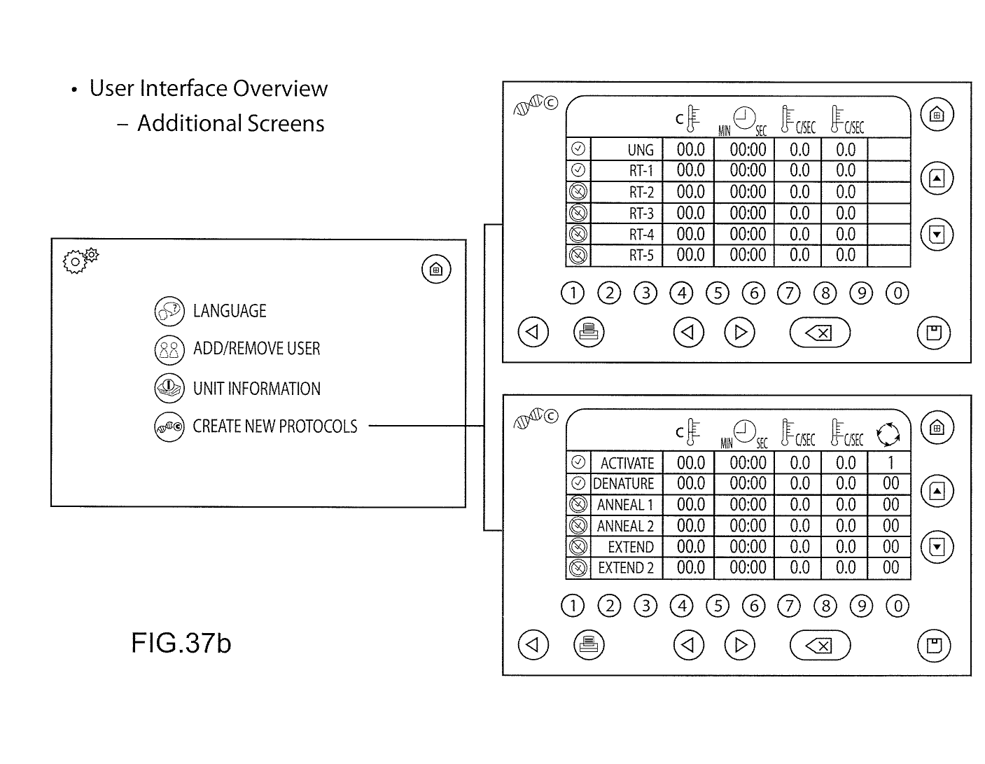

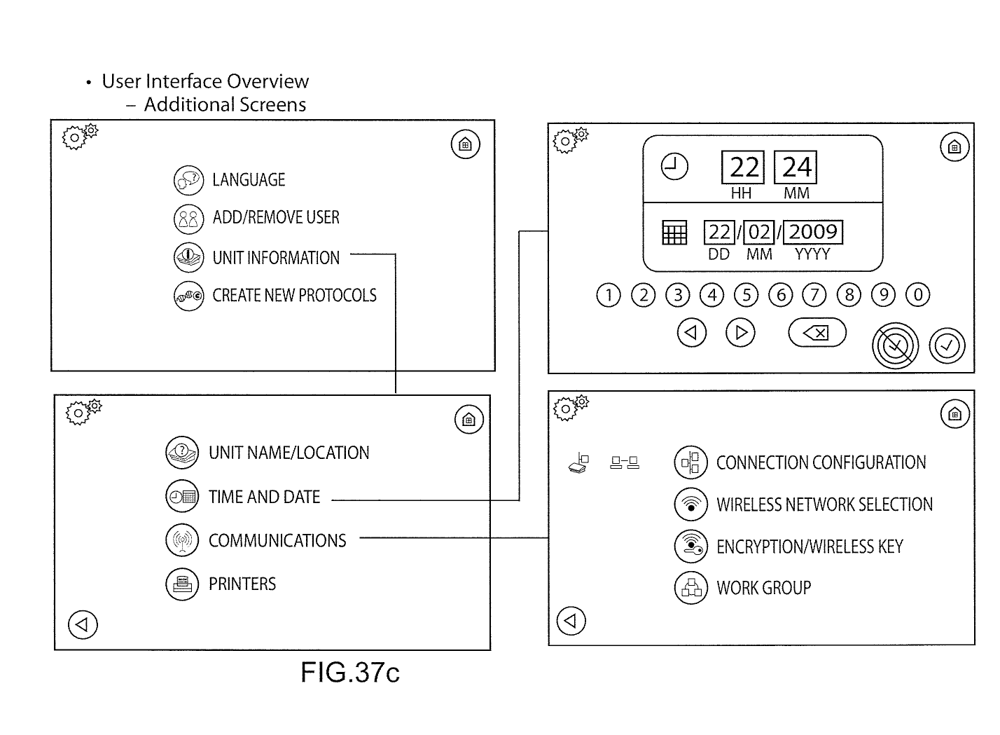

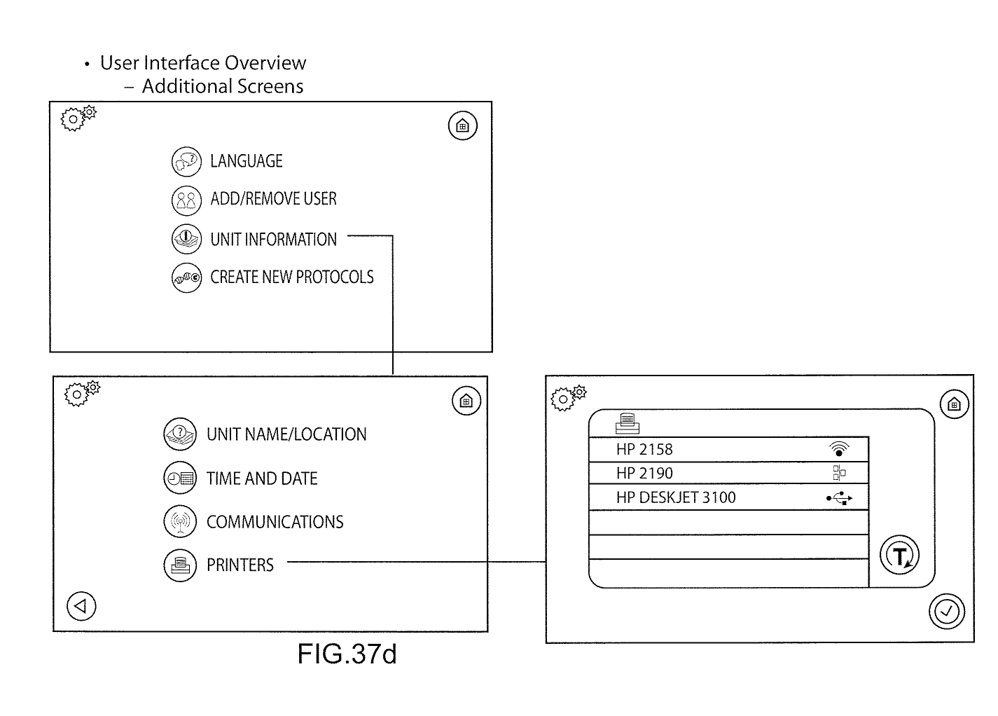

FIGS. 25-37 illustrate example screen shots from the touch screen user interface 208 of PCR device 202. As discussed above, the PCR system 200 provides a fully integrated PCR system that does not require the use of additional computers. The results of the PCR test procedure can be initiated and monitored directly on the touch screen user interface 208. As discussed above, the PCR device 202 can also be configured to communicate with a central monitoring station (e.g., see FIGS. 2 and 3) and/or with other PCR devices.

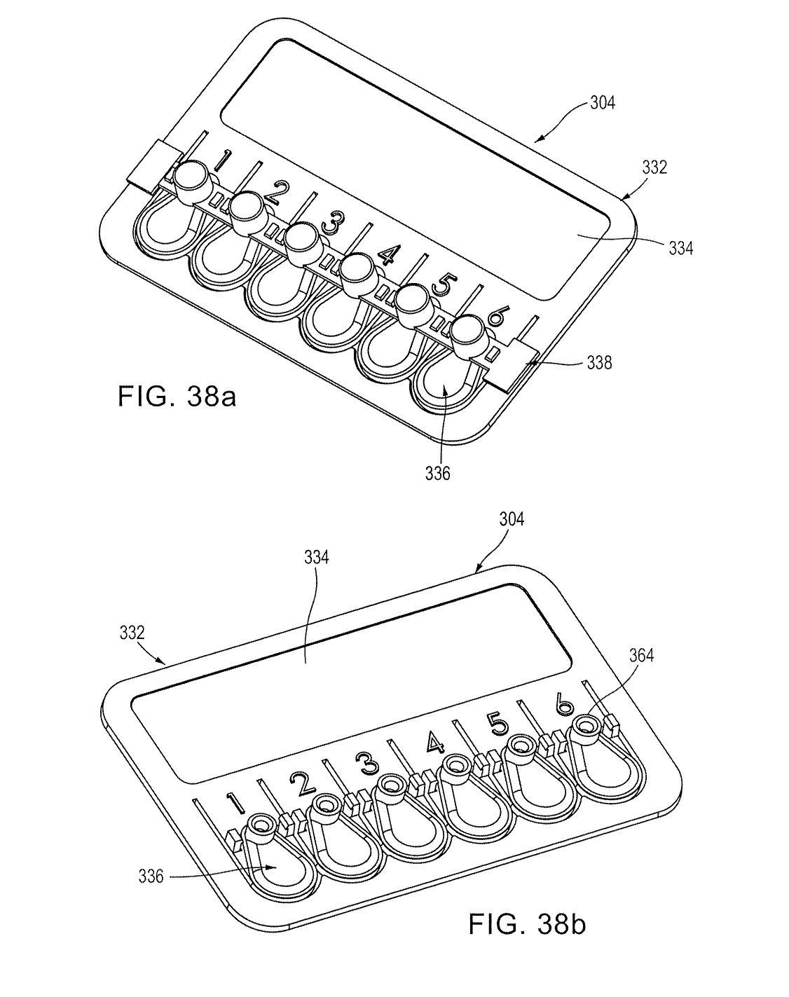

FIGS. 38-40 illustrate another embodiment of a disposable cartridge. A disposable cartridge 304 can be constructed similar to and include the same or similar features as described above for cartridge 304. The cartridge 304 includes a base 332, covers 336 and a cap member 338. The base 332 includes reaction wells 330 and a bar code mounting surface 334. In this embodiment, the base 332 defines slots 340 that do not extend to an outer edge at the bottom of the base 332. The slots 340 in this configuration can provide a different degree of flexibility of the cartridge 304 than with cartridge 204, depending on the material, the thickness, and other factors. The base 332 also includes a snap feature between the reaction wells 330 in the form of posts 327 with hooks configured to be received through corresponding openings (not shown) in the cap member 338.

The covers 336 define a fill port 364 and a see-through optical read area 378. The covers 336 can be ultrasonically bonded to the base 332 as described above. The cap member 338 includes arms 388 disposed between caps 386 that are configured to be disposed over the covers 336. Also shown are numbers that can be disposed over the reaction wells as identifying indicia.

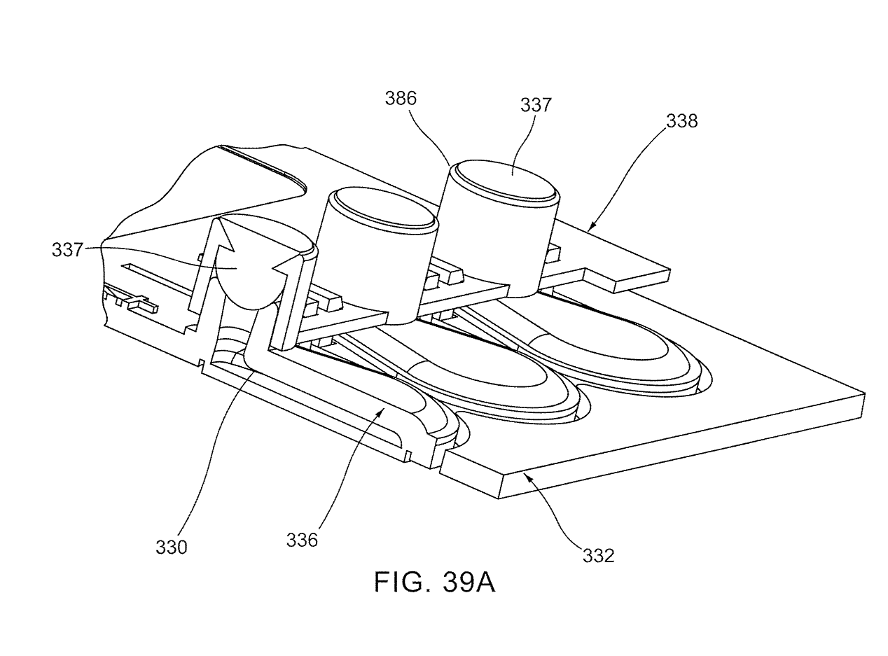

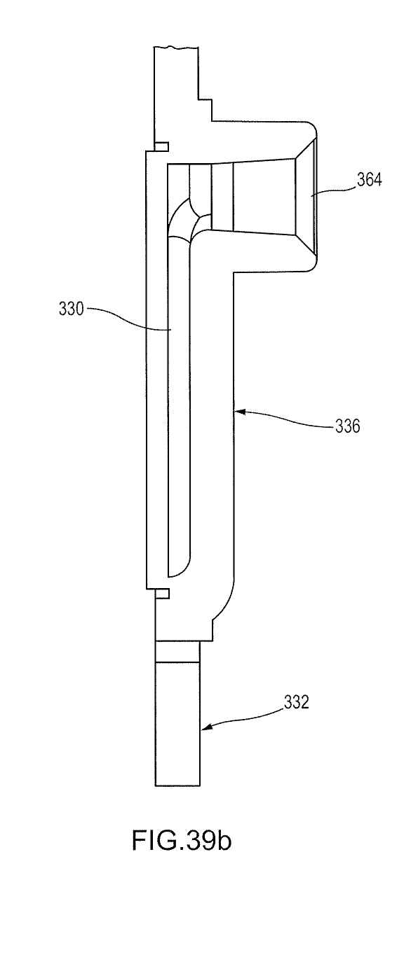

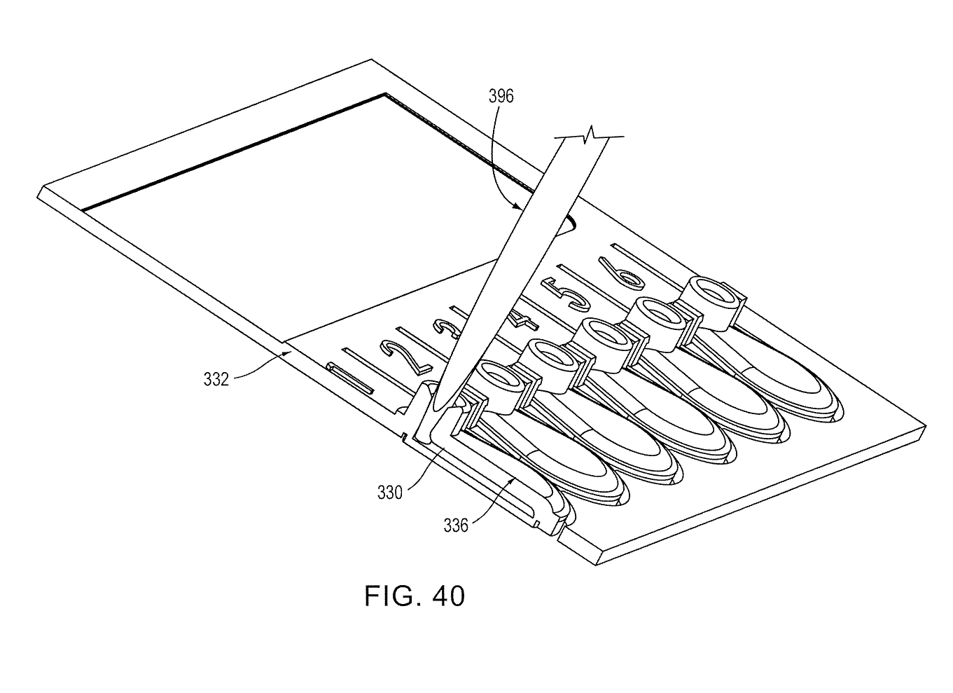

FIG. 39a illustrates a cross-section view of a portion of the cap member 336, a cover 336 and a reaction well 332; and FIG. 39b is a cross-sectional view of a cover 336 and a reaction well 330. As shown in FIGS. 38a and 39a, an over-molded seal 337 is disposed within the caps 386 of the cap member 338. FIG. 40 illustrates a sample delivery device 396 inserted into the fill port 364. The cartridge 304 can be filled, for example, while laying flat (e.g. in a horizontal orientation).

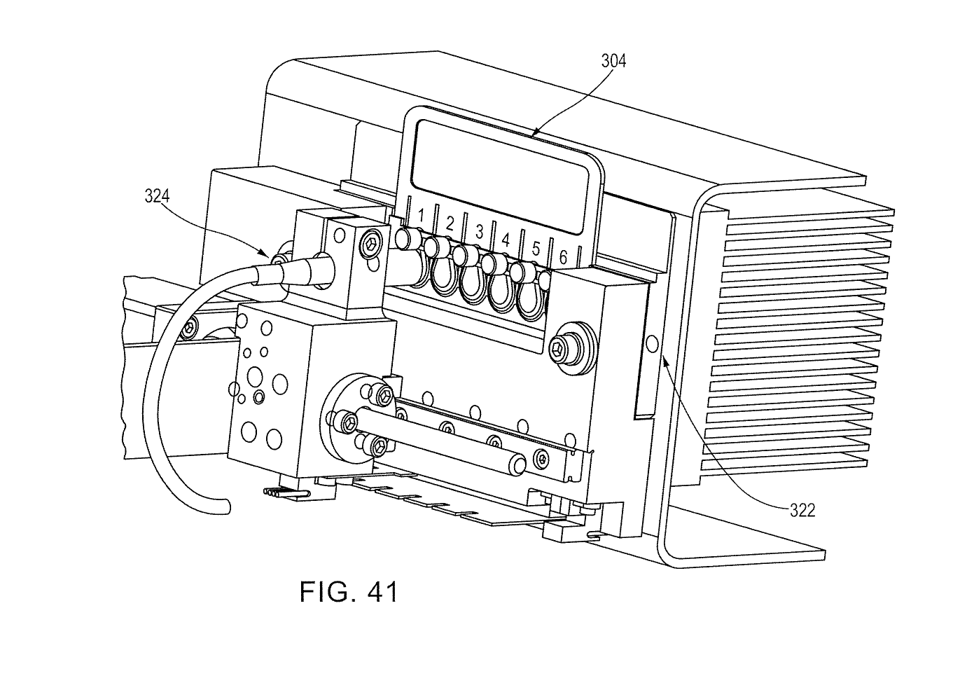

FIG. 41 illustrates the disposable cartridge 304 disposed within a PCR system 300 that includes a PCR monitoring and detection device 302 (only a portion of a PCR device 302 is shown). The PCR device 302 also includes an optical scanning device 324 and a thermal cycler 322. As shown in FIG. 41, the disposable cartridge 304 is positioned within the PCR device 302 in a vertical orientation between the optical scanning device 324 and the thermal cycler 322. Although not specifically described with reference to this embodiment, the PCR device 302 can also include any of the same features, and function in the same or similar manner as described above for previous embodiments.

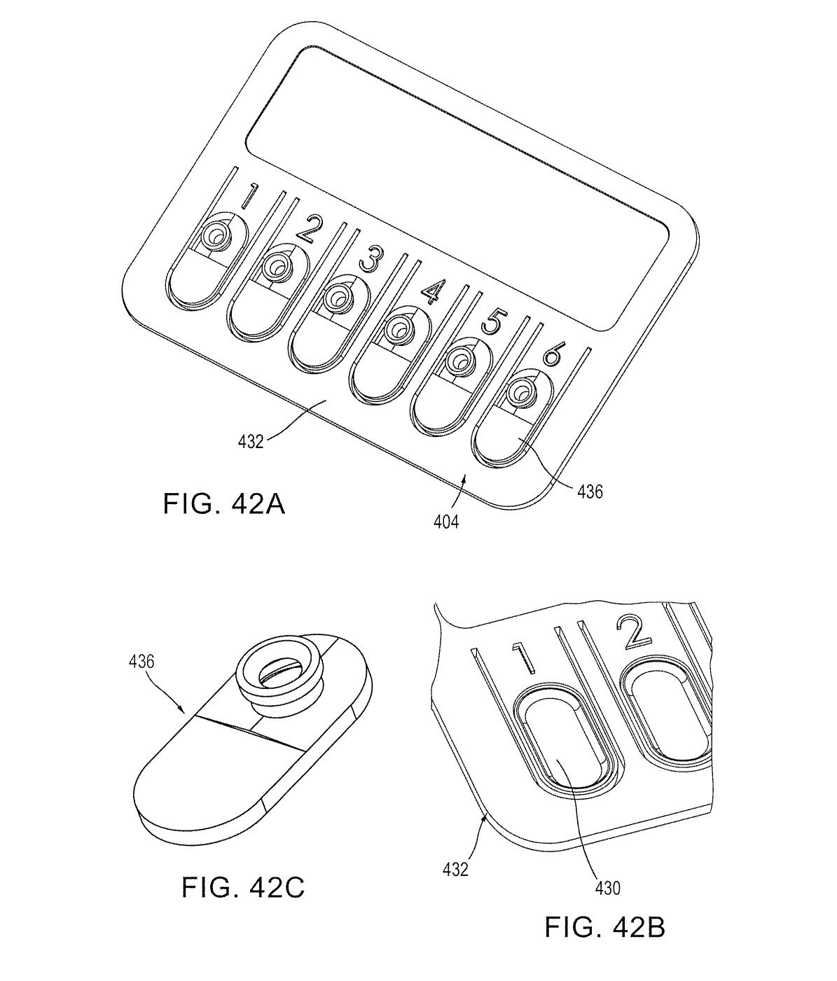

FIGS. 42a-42c illustrate a disposable cartridge according to another embodiment. A disposable cartridge 404 includes a base 432 defining multiple reaction wells 430, and multiple covers 436 configured to be bonded to the base 432. The disposable cartridge 404 can also include caps or a cap member (not shown) to cover the fill ports on the covers 436 as described previously for other embodiments. The disposable cartridge 404 can be configured to perform the same or similar functions as described for previous embodiments and can be received in a PCR device as described herein. For example, the disposable cartridge 404 can be disposed within a PCR device in a vertical orientation.

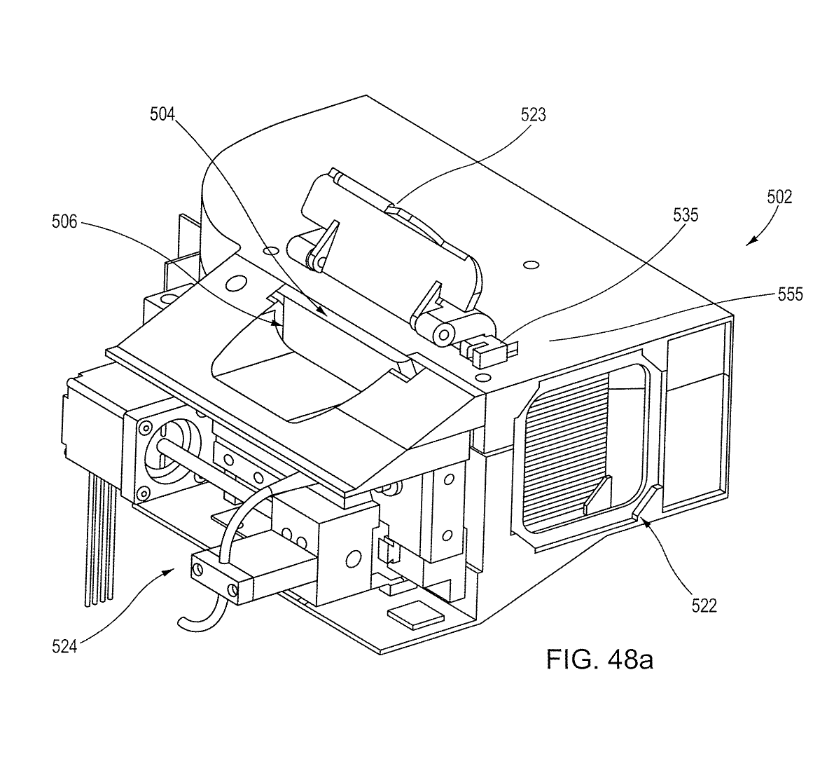

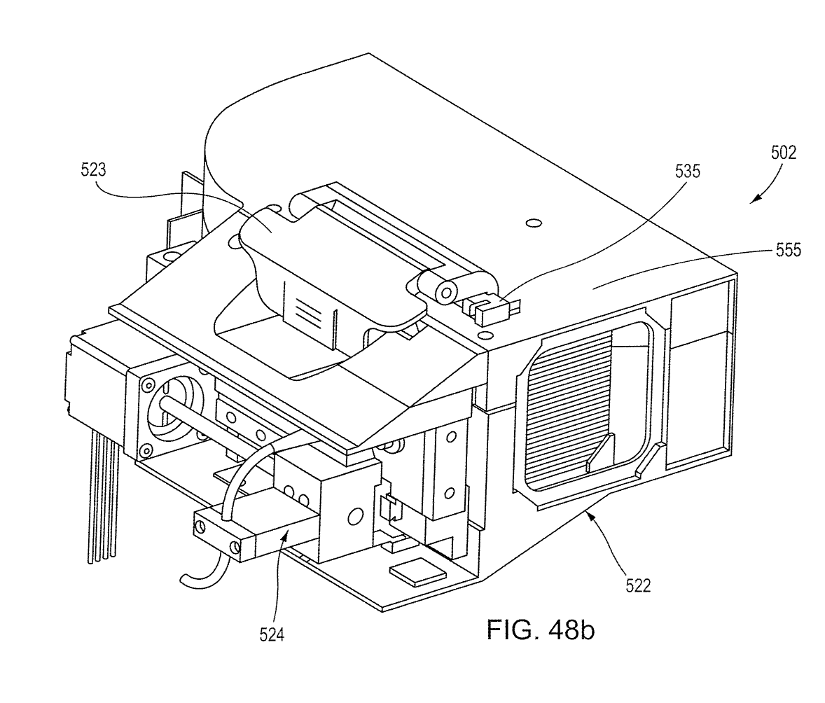

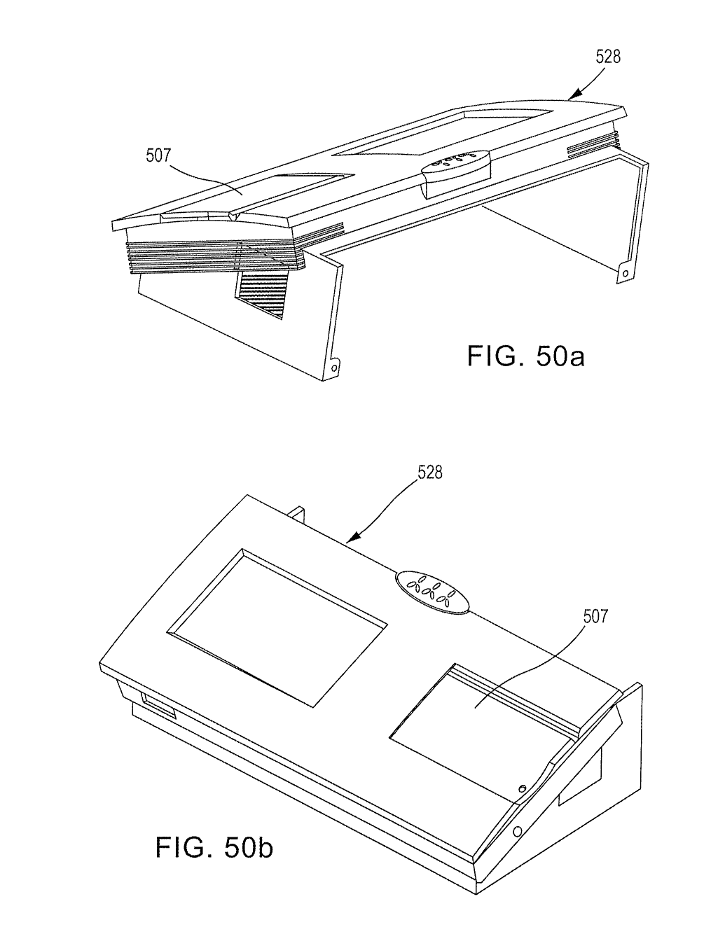

FIGS. 48a through 50b illustrate another embodiment of a PCR device. A PCR device 502 includes a housing 528 (see FIGS. 50a and 50b), a thermal cycler 522, an optical scanning device 524 and is configured to receive a disposable cartridge 504 as described herein. Although not discussed in detail with reference to this embodiment, the PCR device 502 can include any of the features described herein for other embodiments of a PCR device and can function in the same or similar manner. As shown in FIGS. 48a and 48b, in this embodiment, the PCR device 502 includes an optical sensor 535 disposed on a housing 555 of the thermal cycler 522. The optical sensor 535 can be configured to indicate when the disposable cartridge 504 has been properly inserted into a cartridge receiving portion 506 of the PCR device 502.

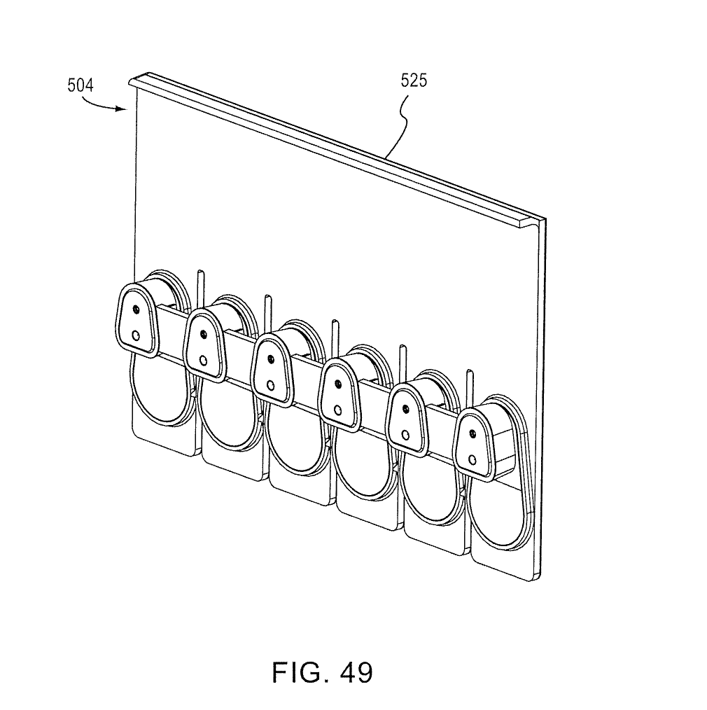

This embodiment also illustrates an inner door 523 hingedly coupled to the housing 555 of the thermal cycler device 522. The inner door 513 can be configured to clamp onto the disposable cartridge 504 when it is inserted into the cartridge receiving portion 506 of the PCR device 502. As shown in FIGS. 50a and 50b, the housing 528 of the PCR device 602 can also include an outer door 507 as described above (see e.g., PCR device 202). FIG. 48a illustrates the inner door 523 in an open position, and FIG. 48b illustrates the inner door 523 in a closed position and clamped onto the cartridge 504. FIG. 49 illustrates the disposable cartridge 504. In this embodiment, the cartridge 504 includes an optional pull-tab 525 at the top. The cartridge 504 can also include other components and features as described herein for other embodiments of a disposable cartridge.

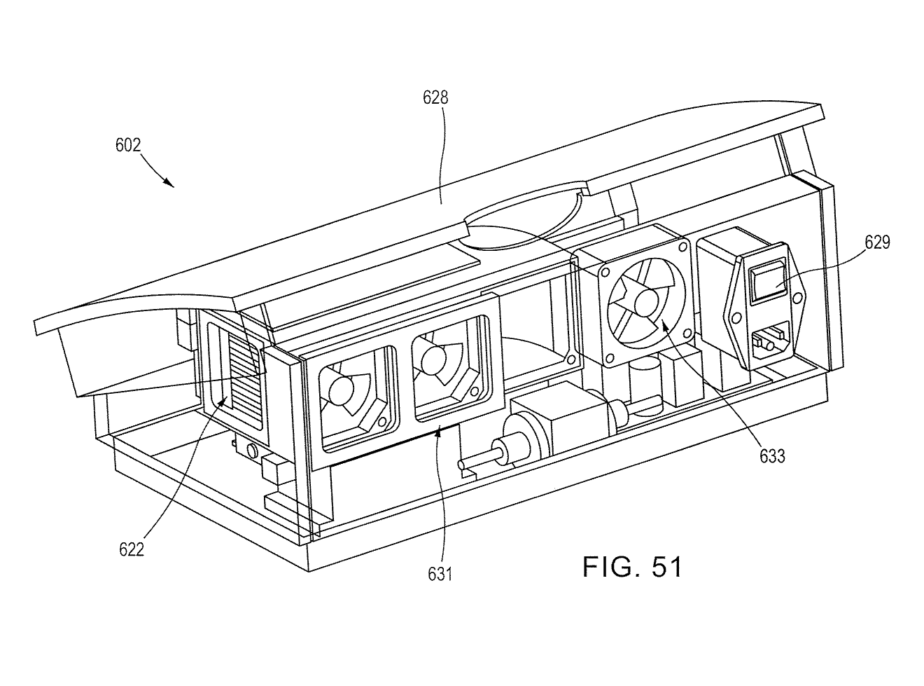

FIG. 51 illustrates another embodiment of a PCR device. A PCR device 602 includes a housing 628 shown in see-through to enable viewing inside the housing 628. Also visible are a thermal cycler 622 and its associated fans 631, and fan 633 for cooling the interior and electronics. A power switch 629 is also shown. Although not specifically described with reference to this embodiment, the PCR device 602 can include any of the components and function in the same or similar manner as described herein for other embodiments.

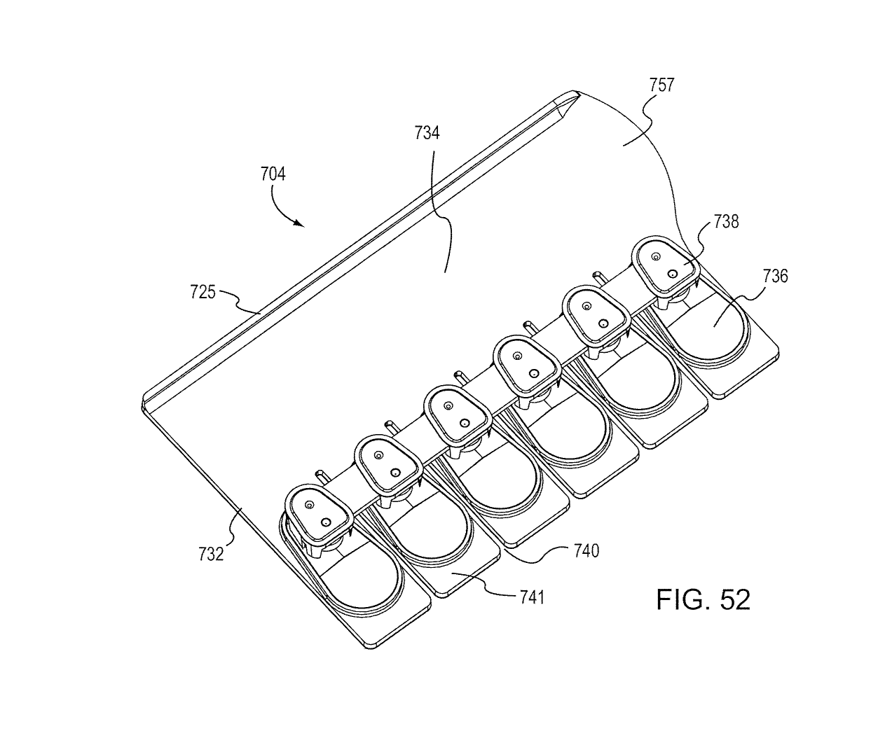

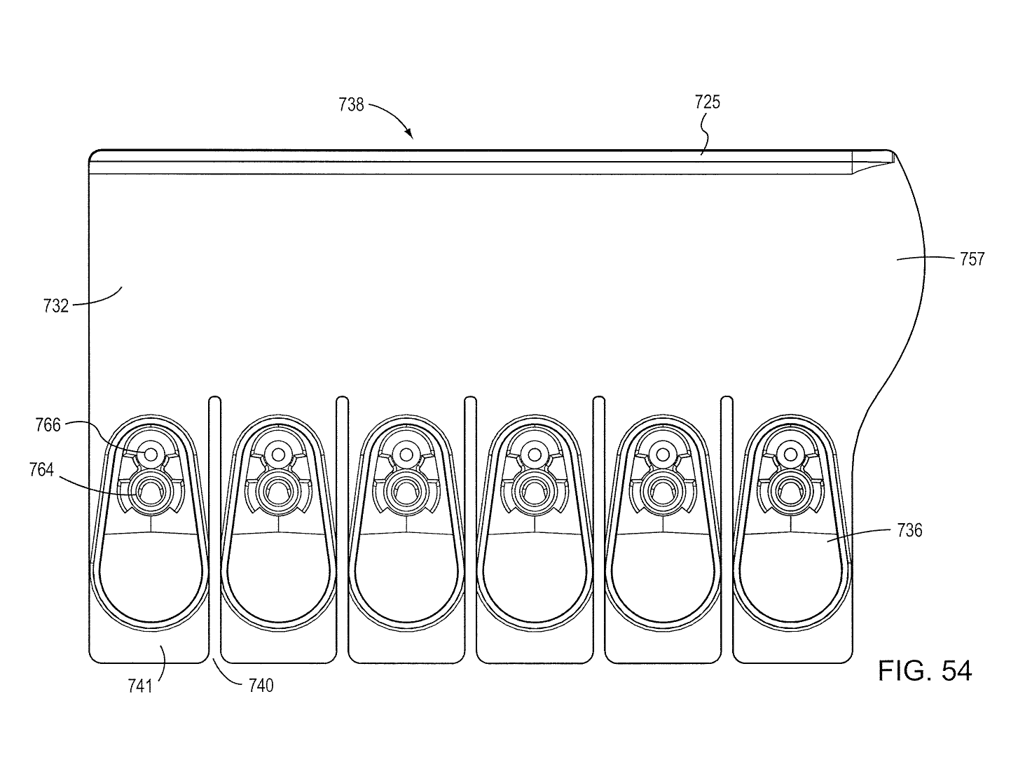

FIGS. 52-56 illustrate another embodiment of a disposable cartridge. A disposable cartridge 704 includes a base 732, covers 736 and a cap member 738. Although not necessarily discussed in detail below with reference to this embodiment, it should be understood that the disposable cartridge 704 can include any of the features and functions described herein for other embodiments. The base 732 includes reaction wells (not shown) and a bar code mounting surface 734. The base 732 also defines slots 740 that define flexible fingers 741 and a pull-tab 725 at the top as described above.

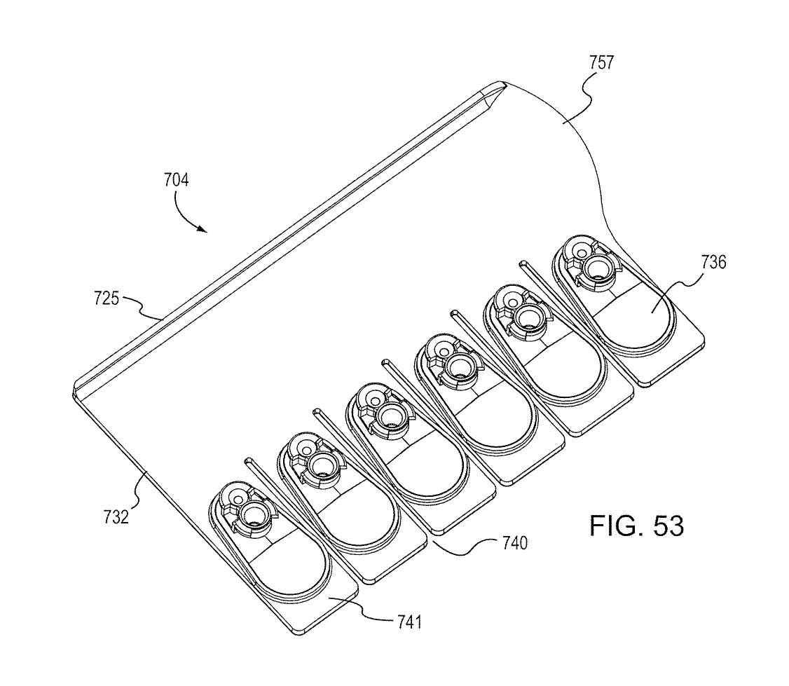

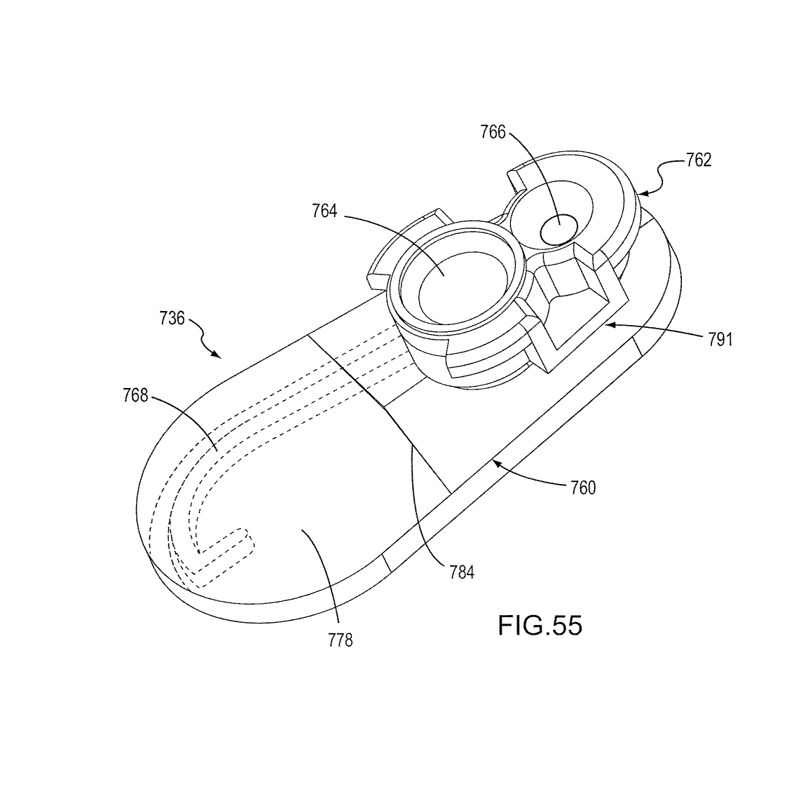

As shown in FIGS. 53-55, the covers 736 each include a body portion 760 and a raised portion 762. The body portion 760 defines a vent tube 768 and a flat, clear wall 778 that is disposed over the associated reaction well when coupled to the base 732. A fill line 784 is disposed on the wall 778 as described above for cartridge 204. The raised portion 762 defines a fill port 764, and an exit port 766 that is in communication with the vent tube 768. The covers 736 can be ultrasonically bonded and/or adhesively bonded to the base 732 as described above. As shown, for example, in FIG. 56, the cap member 738 includes arms 788 disposed between caps 786 that are configured to be disposed over the covers 736. The cap member 738 also includes a neck portion 789 configured to be inserted into the fill port 764 of the covers 736 when coupled thereto, and guides 790 configured to provide a snap-fit to a mating coupling feature 791 on the covers 736 (see FIG. 55).

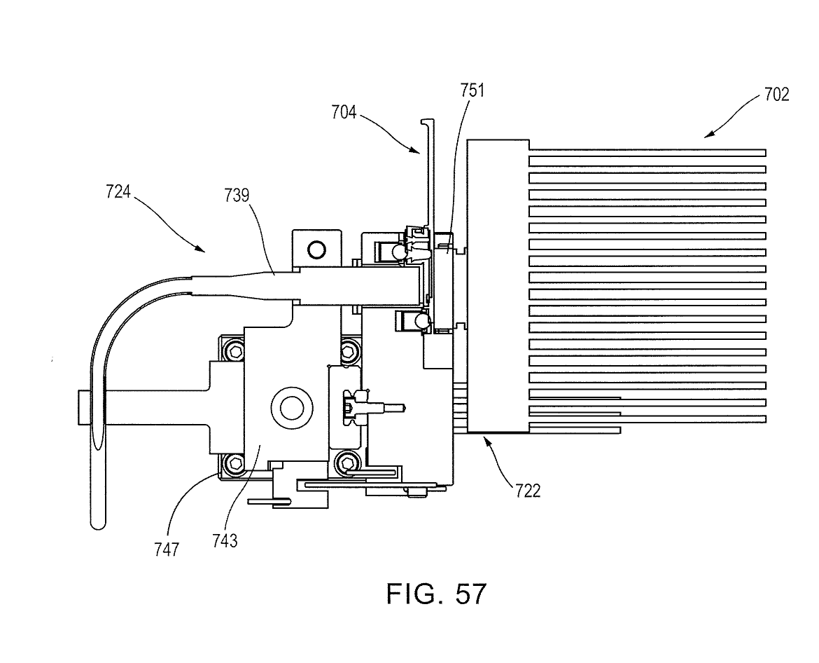

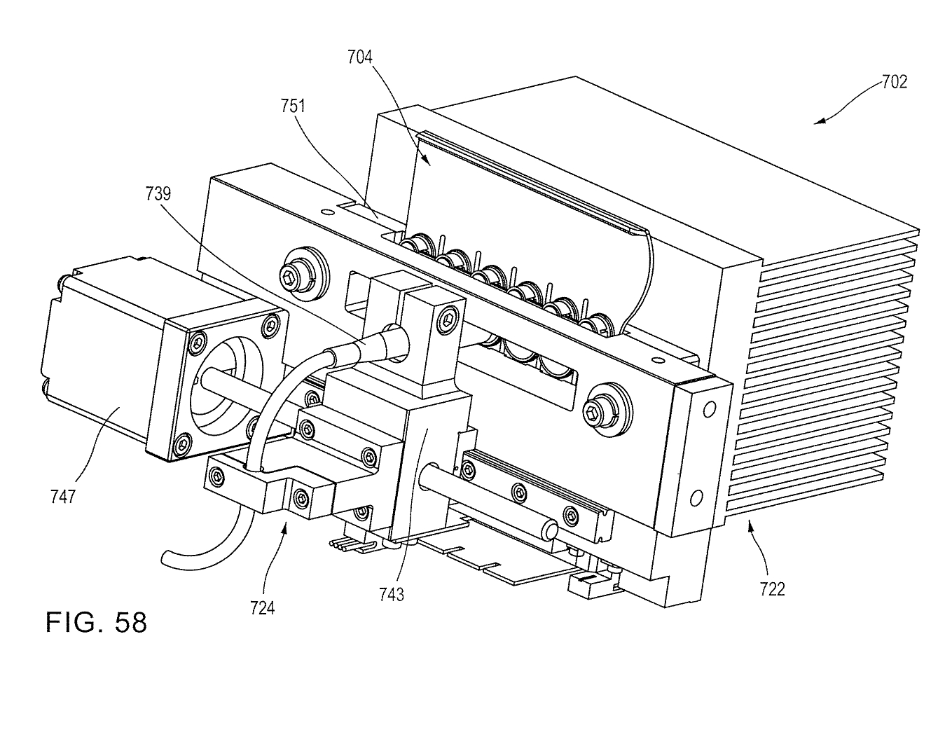

The disposable cartridge 704 can be received in a PCR device as described herein. For example, as shown in FIGS. 57 and 58 the disposable cartridge 704 can be disposed within a PCR device 702 in a vertical orientation (only a portion of a PCR device 702 is shown). The PCR device 702 includes an optical scanning device 724 and a thermal cycler 722. The thermal cycler 722 includes a heat plate 751. The optical scanning device 724 includes a read optic 739 mounted on a movable carriage 243. A stepper motor 747 is operatively coupled to the carriage 743 to move the carriage 743 back and forth in front of the disposable cartridge 704. As discussed previously for other embodiments, the cartridge 704 is disposed between the heat plate 751 and the optical scanning device 724 in a vertical orientation such that the read optic 739 can be translated (by the carriage 743) across the reaction wells of the disposable cartridge 704. Although not specifically described with reference to this embodiment, it should be understood that the PCR device 702, can also include any of the same features, and function in the same or similar manner, as described herein for other embodiments. The thermal cycler 724 and optical scanning device 724 can also include other features and functions not specifically discussed with reference to this embodiment.

In this embodiment, the base 732 also defines a side tab 757 on which a sensor flag (not shown) can be disposed (see e.g., cartridge 804 discussed below). The sensor flag can be configured to trigger a sensor in the PCR device 702 to indicate to the associated optical scanning device 724 that the disposable cartridge 704 is in a proper position.

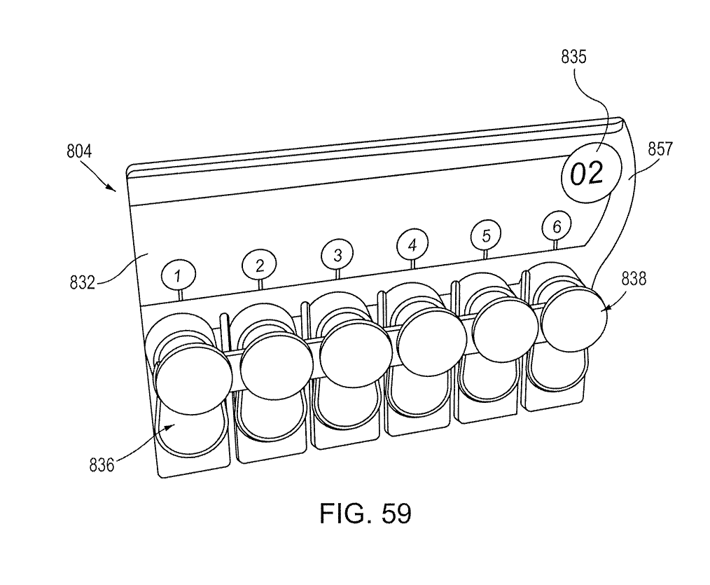

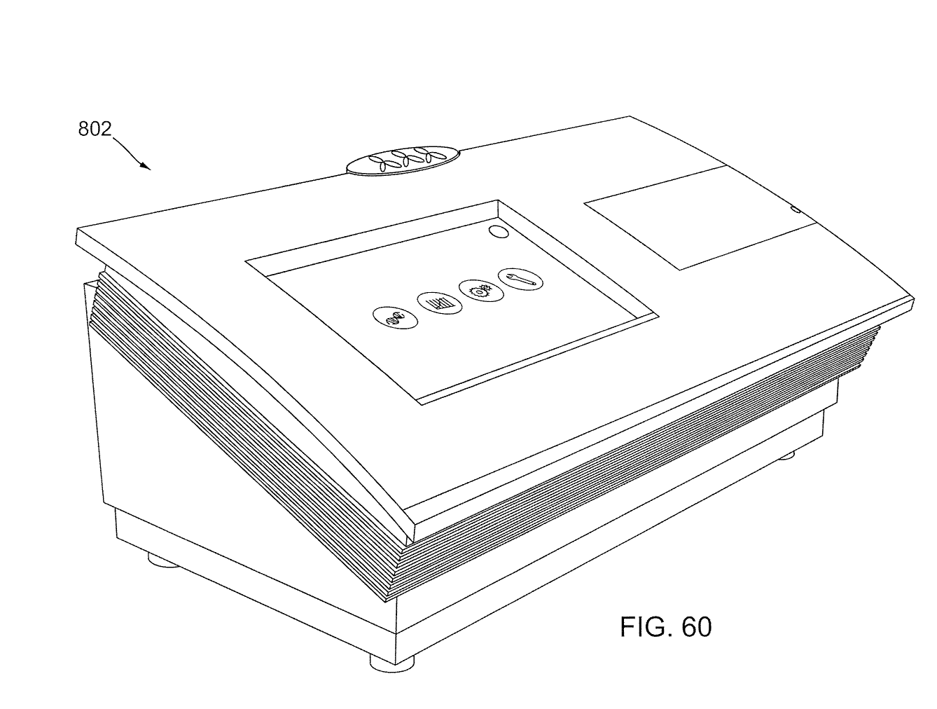

FIG. 59 illustrates a disposable cartridge according to another embodiment. A disposable cartridge 804 includes a base 832 defining multiple reaction wells 830, and multiple covers 836 configured to be bonded to the base 832. The disposable cartridge 804 also includes a cap member 838 to cover the fill ports on the covers 836 as described previously for other embodiments. The disposable cartridge 804 can include the same or similar components, and/or perform the same or similar functions, as described for other embodiments and can be received in a PCR device as described herein, such as the PCR device 802 shown in FIG. 60. For example, the disposable cartridge 804 can be disposed within a PCR device in a vertical orientation. Although not discussed in detail with reference to this embodiment, the PCR device 802 can include any of the features described herein for other embodiments of a PCR device and can function in the same or similar manner.

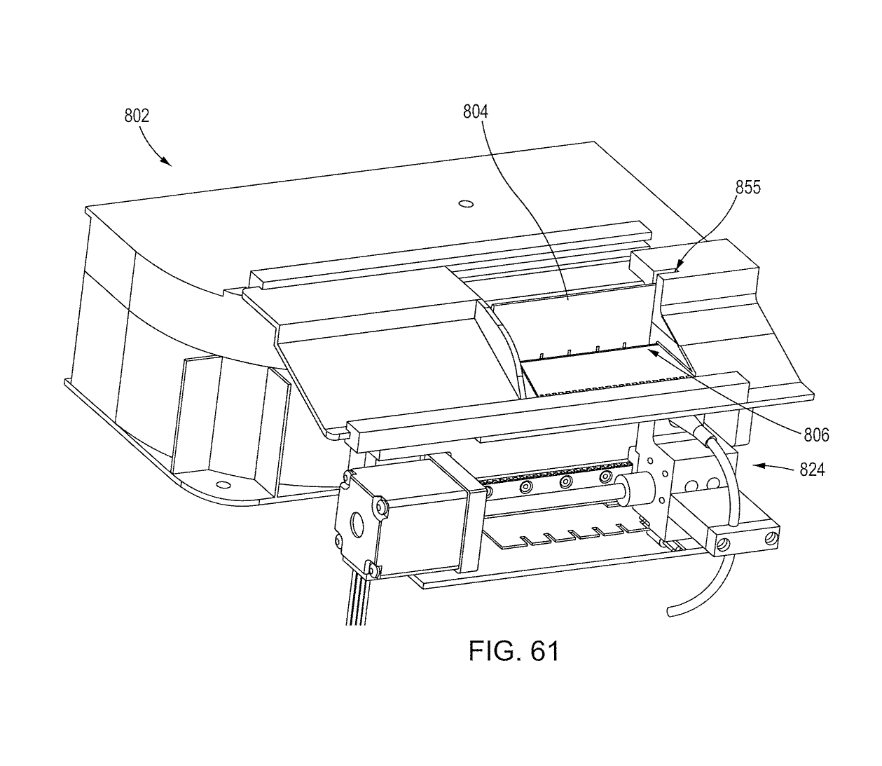

Disposable cartridge 804 also includes an optical sensor flag 835 disposed on a side tab 857 defined by the base 832. The sensor flag 835 can be configured to trigger a sensor in the PCR device 802 to indicate to the associated optical scanning device that the disposable cartridge 804 is in a proper position. For example, FIG. 61 is an example illustration of an interior of the PCR device 802, including an optical scanning device 824. An optical sensor (not shown) can be disposed within the PCR device 802, for example, in a cartridge receiving portion 806. The sensor flag 835 can trigger the optical sensor when the cartridge 804 is positioned within the cartridge receiving portion 806 and communicates to the optical scanning device 824 that the cartridge 804 is in a proper position.

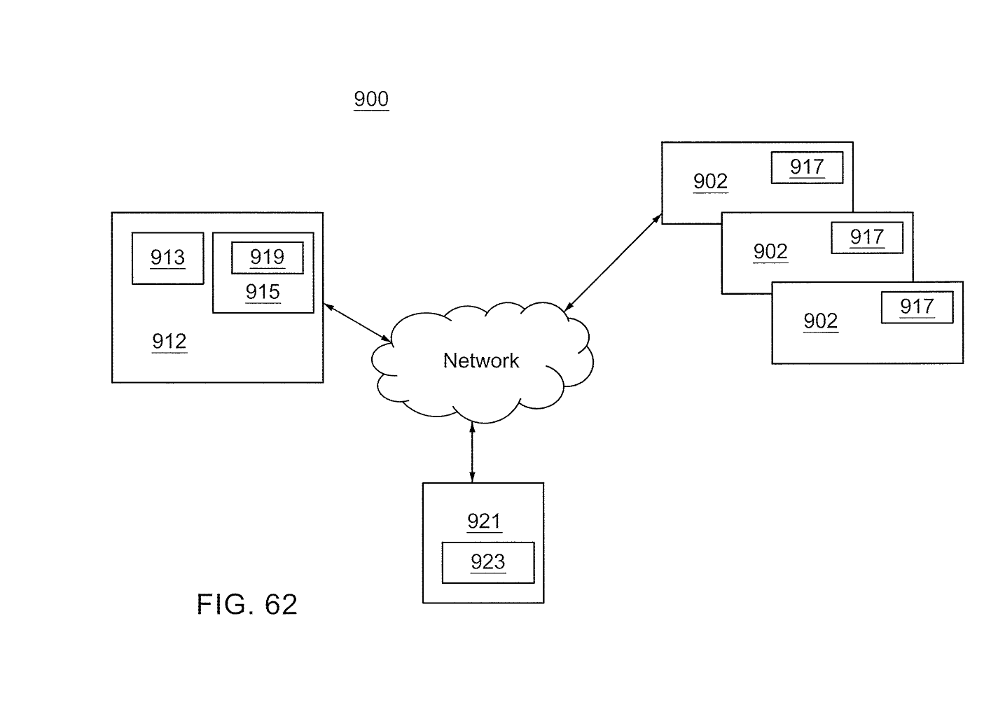

Referring back to FIGS. 3 and 4, as described above, a PCR system described herein can include one or more PCR devices that are in communication with a central monitoring station. FIG. 62 is a schematic illustration of a system according to an embodiment. A PCR system 900 includes central processing station 912 and multiple PCR device 902. The central monitoring station 912 can be a computer that includes a processor-readable medium 913 storing code representing instructions to cause a processor 915 to perform a process. The processor 915 can be in communication with one or more processors 917 associated with the PCR devices 902 via a network, such as the Internet. In some embodiments, the processors 917 can be in communication with the processor 915 through a server 921, via a network such as the Internet. The server 921 can also include a processor-readable medium 923 storing the code and can provide access to the code via a network.

The processor 915 is described in more detail below, but it should be understood that the processors 917 can be similarly configured. The processor 915 can be, for example, a commercially available personal computer, or a less complex computing or processing device that is dedicated to performing one or more specific tasks. For example, the processor 915 can be a terminal dedicated to providing an interactive graphical user interface (GUI). The processor 915, can be a commercially available microprocessor. Alternatively, the processor 915 can be an application-specific integrated circuit (ASIC) or a combination of ASICs, which are designed to achieve one or more specific functions, or enable one or more specific devices or applications. In yet another embodiment, the processor 915 can be an analog or digital circuit, or a combination of multiple circuits.

The processor 915 can include a memory component 919. The memory component 919 can include one or more types of memory. For example, the memory component 919 can include a read only memory (ROM) component and a random access memory (RAM) component. The memory component 919 can also include other types of memory that are suitable for storing data in a form retrievable by the processor 915. For example, electronically programmable read only memory (EPROM), erasable electronically programmable read only memory (EEPROM), flash memory, as well as other suitable forms of memory can be included within the memory component 919. The processor 915 can also include a variety of other components, such as for example, co-processors, graphic processors, etc., depending upon the desired functionality of the code.

The processor 915 is in communication with the memory component 919, and can store data in the memory component 919 or retrieve data previously stored in the memory component 919. The components of the processor 915 can communicate with devices external to the processor 915 by way of an input/output (I/O) component (not shown). According to one or more embodiments of the invention, the I/O component can include a variety of suitable communication interfaces. For example, the I/O component can include, for example, wired connections, such as standard serial ports, parallel ports, universal serial bus (USB) ports, S-video ports, local area network (LAN) ports, small computer system interface (SCCI) ports, and so forth. Additionally, the I/O component can include, for example, wireless connections, such as infrared ports, optical ports, Bluetooth® wireless ports, wireless LAN ports, or the like.

The processor 915 can be connected to a network, which may be any form of interconnecting network including an intranet, such as a local or wide area network, or an extranet, such as the World Wide Web or the Internet. The network can be physically implemented on a wireless or wired network, on leased or dedicated lines, including a virtual private network (VPN).

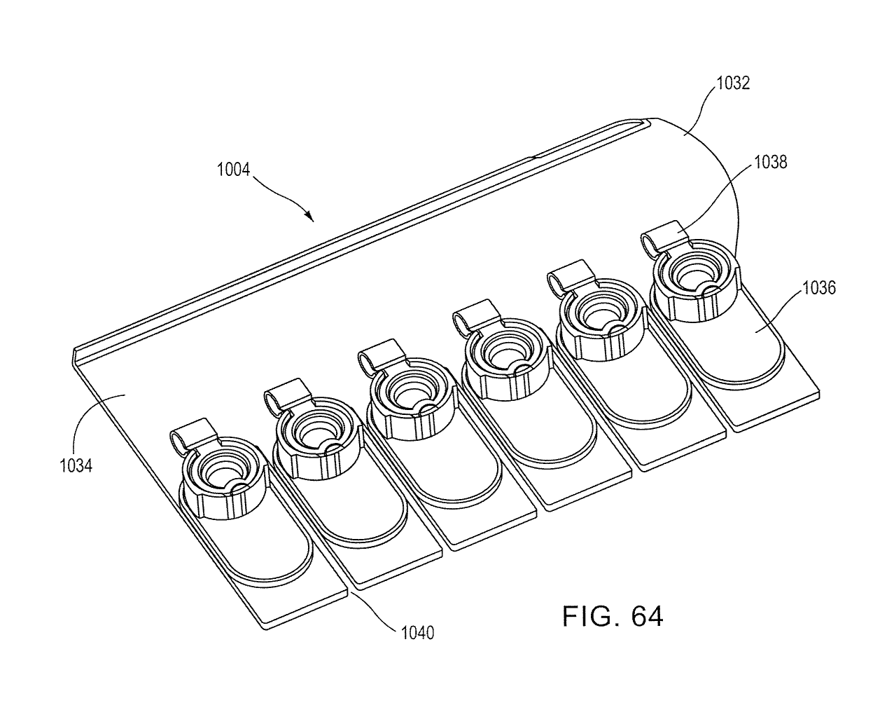

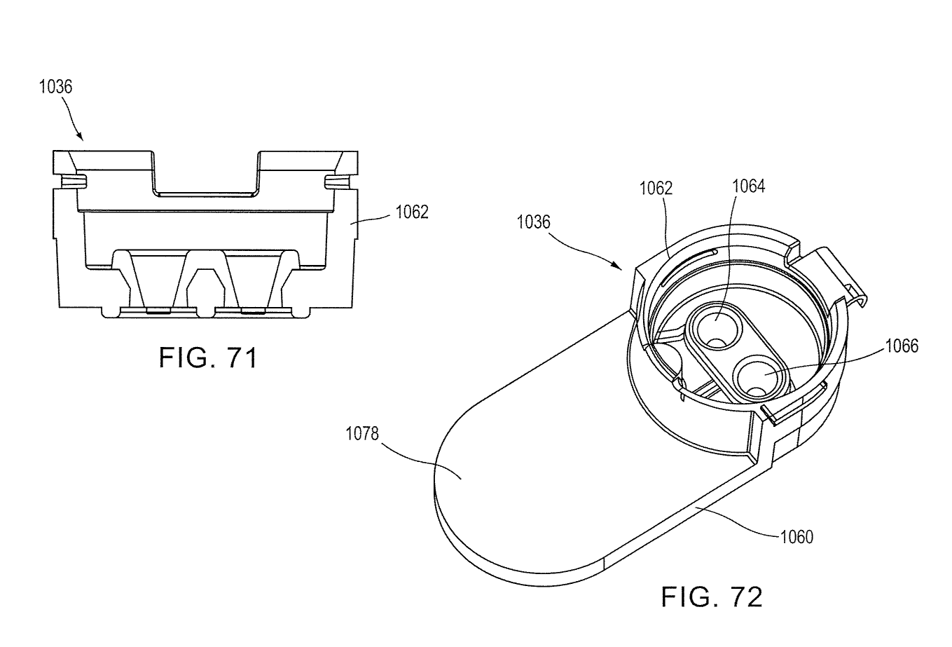

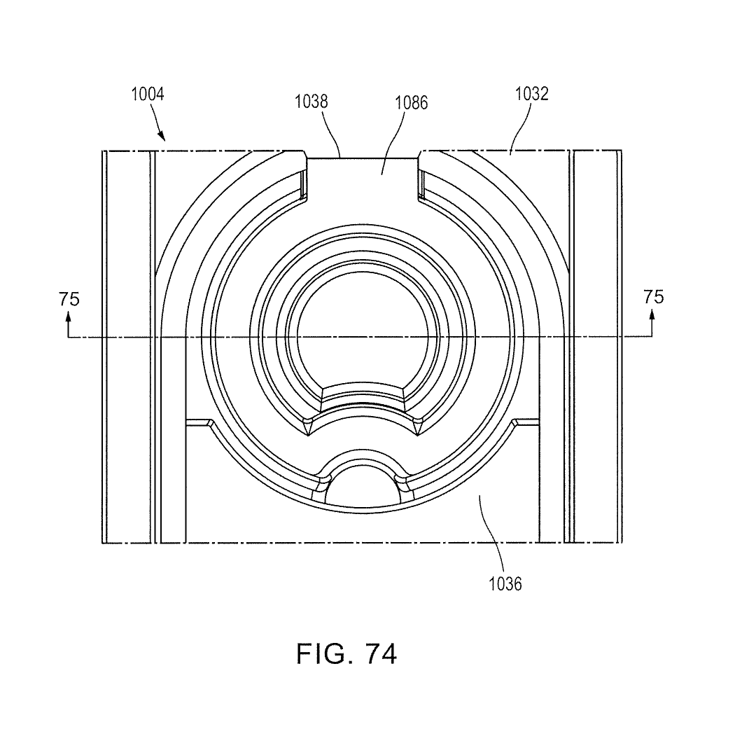

FIGS. 64-75 illustrate another embodiment of a disposable cartridge. A disposable cartridge 1004 includes a base 1032, covers 1036 and multiple cap members 1038. In this embodiment, the cartridge 1004 include separate cover members 1036 that can be hingedly coupled to the covers, and a snap-fit feature to snap-fit the cap members 1038 to the covers 1036. The separate cap members 1038 allows for a single individual well to be filled if desired. Although not necessarily discussed in detail below with reference to this embodiment, it should be understood that the disposable cartridge 1004 can include any of the features and functions described herein for other embodiments.

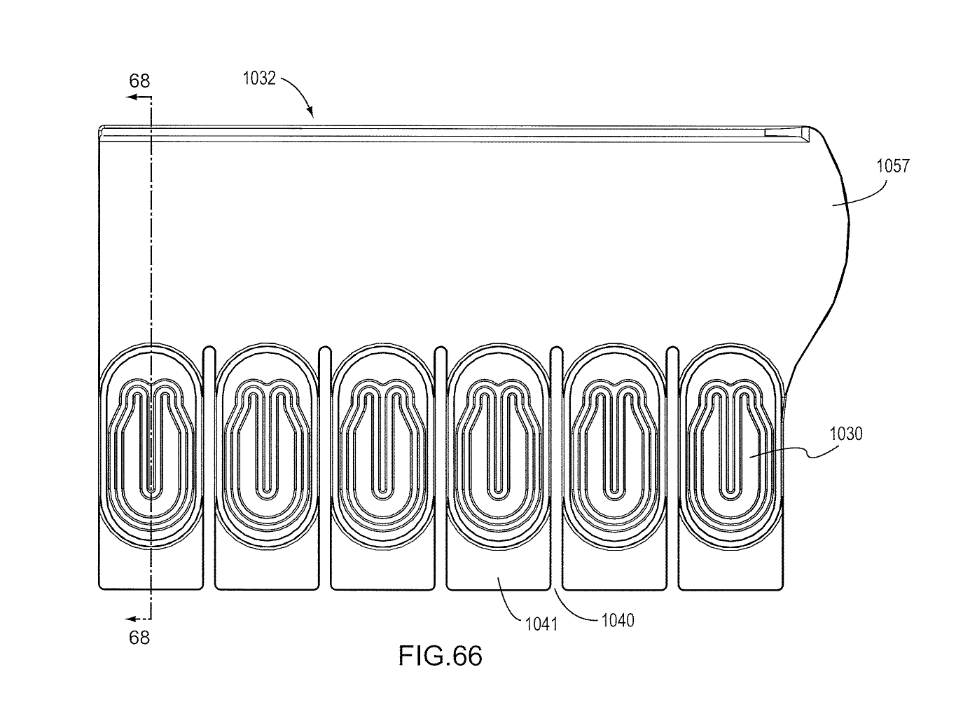

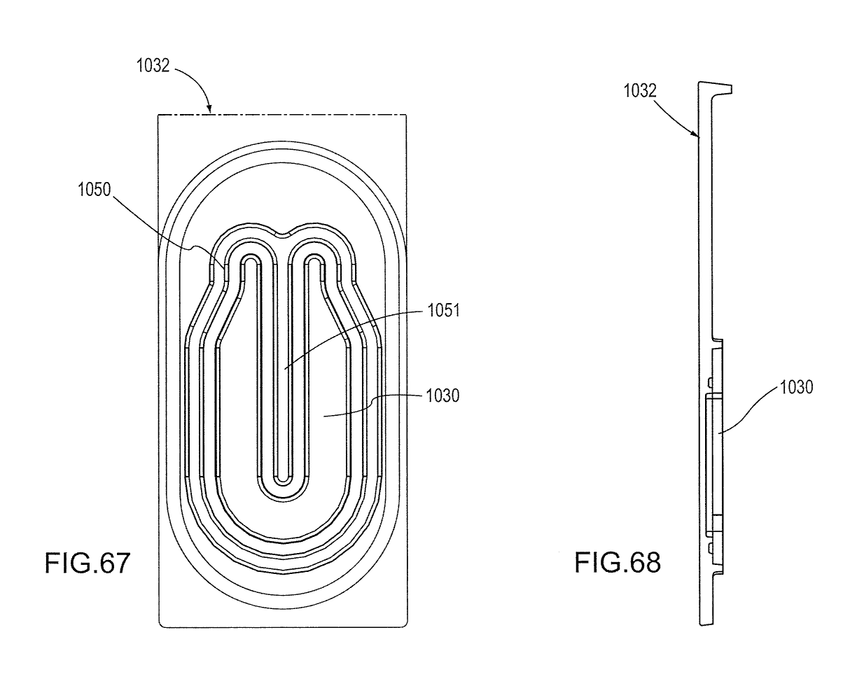

The base 1032 defines reaction wells 1030 (see, e.g., FIG. 66-68) and a surface 1034 to which a bar code can be mounted as described above for previous embodiments. The base 1032 also defines slots 1040 that define flexible fingers 1041 (see, e.g., FIG. 66) and a pull-tab 1025 at the top as described above. The base 1032 can have a wall thickness, for example, of about 1 mm+/−0.05 mm. The surface finish of the base 1032 can be, for example, SP1 A1, and the optical axis length can be, for example, about 3.5 mm for each well. The base 1032 defines channels 1050 (see, e.g., FIG. 67) that can be used for controlled location, indexing, and adhesive coupling of each of the cover members 1036 to its corresponding location on the base 1032. The channels 1050 can have a width, for example, of 0.75 mm and a depth of, for example, 0.30 mm. A tongue portion 1051 of the channel 1050 can have a width of, for example, 0.50 mm and a depth of, for example, 0.25 mm.

The covers 1036 and/or the caps 1038 can include a compressible material (e.g., an injection molded elastomeric structure, for example). The compressible material reduces resistance to insertion of the cartridge 1004 in a PCR device, provides an improved seal between the covers 1036 and the caps 1038, and/or reduces transmission of forces generated by the PCR device during insertion of the cartridge to the cap member 1038. The compressible material may be attached to the covers 1036 or the caps 1038 in any suitable manner.

The base 1032 also defines a side tab 1057 on which a sensor flag (not shown) can be disposed (see e.g., cartridge 804 discussed above). The sensor flag can be configured to trigger a sensor in a PCR device to which the cartridge 1004 is being used to indicate to the associated optical scanning device of the PCR device that the disposable cartridge 1004 is in a proper position.

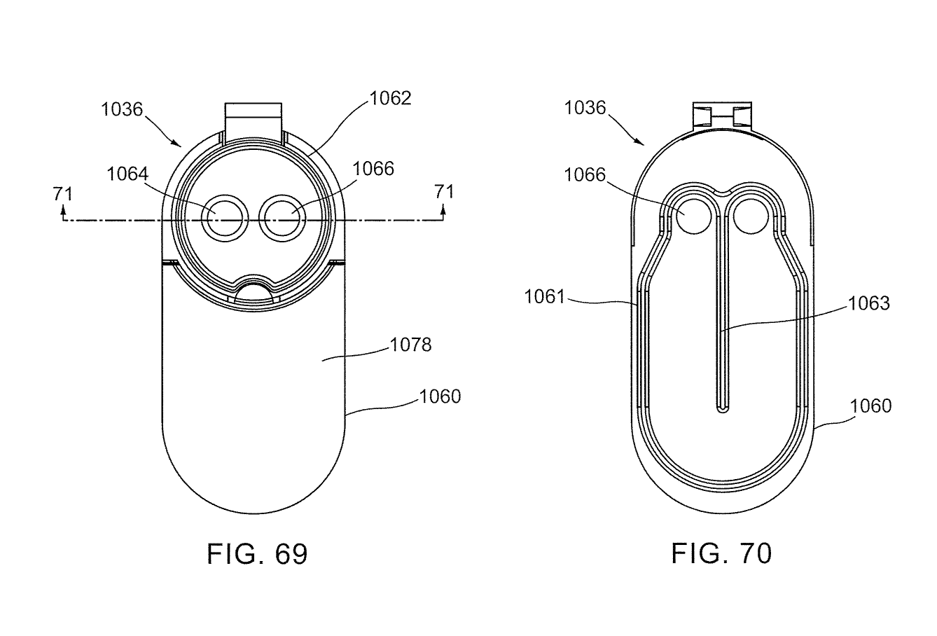

As shown in FIGS. 69-72, the cover members 1036 each include a body portion 1060 and a raised portion 1062. The body portion 1060 defines a vent tube 1068 and a flat, clear wall 1078 that is disposed over the associated reaction well when coupled to the base 1032. A fill line (not shown) can be disposed on the wall 1078 as described above for cartridge 204. The raised portion 1062 defines a first fill port 1064 and a second fill port 1066. The first and second fill ports 1064 and 1066 provide for larger venting, and therefore, reduce the likelihood of clogging, compared to cartridge 204. The covers 1036 can be adhesively bonded to the base 1032. Alternatively, the covers 1036 can be ultrasonically bonded to the base 1032 as described above for previous embodiments. The cover members 1036 can have a surface finish of, for example, SP1 A1 for the read area over the reaction wells 1030. The cover members 1036 have an adhesive channel 1061 that can interface with channel 1050 of the base 1032 to adhesively couple the cover members 1036 to the base 1032. The channels 1061 can have a width, for example, of 0.75 mm and a depth of, for example, 0.30 mm, and a tongue portion 1063 of the channel 1061 can have a width of, for example, 0.50 mm and a depth of, for example, 0.25 mm.

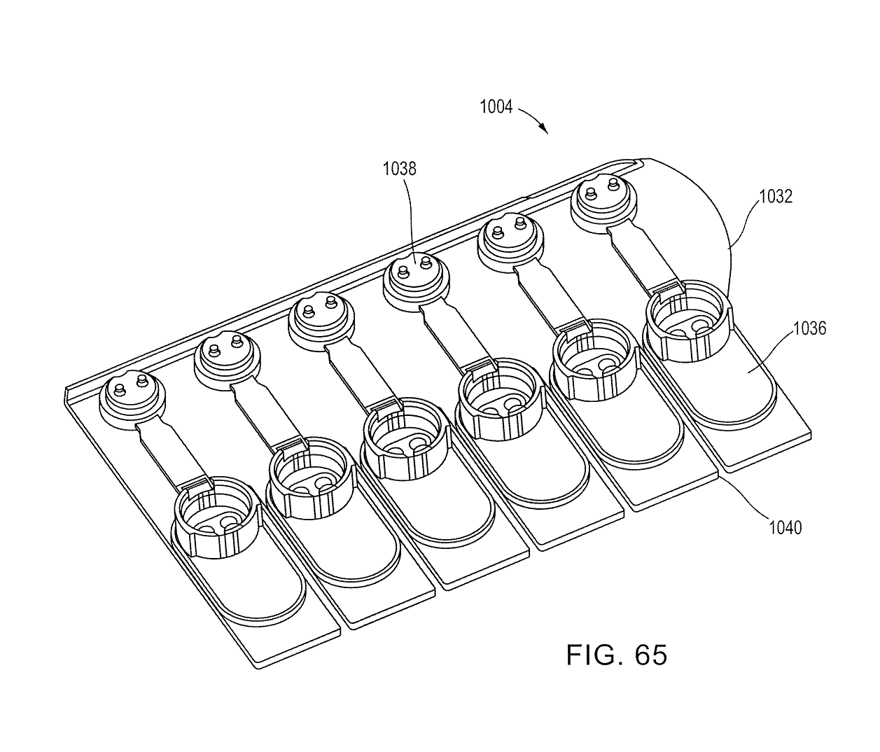

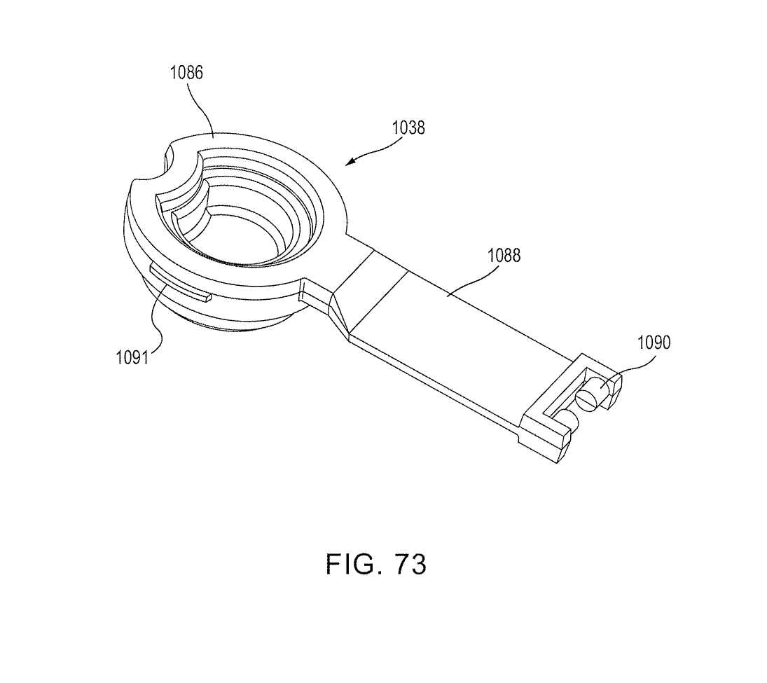

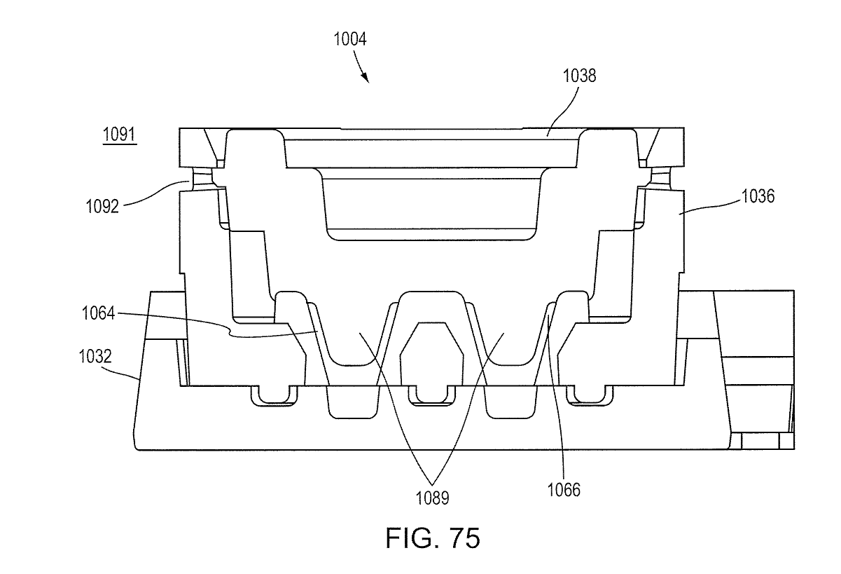

As shown, for example, in FIG. 73, each of the cap members 1038 include a cap arm portion 1088 extending from a cap portion 1086 that is configured to be disposed over and coupled to the covers 1036. The cap members 1038 include extension portions 1089 configured to be inserted into the first fill port 1064 and the second fill port of the covers 1036 when coupled thereto as shown in FIG. 75. The cap members 1038 also include a first coupling portion 1090 (see FIG. 73) configured to provide a hinged coupling to the covers 1036 to allow the cap members 1038 to be moved between a closed position (as shown in FIG. 64) and an open position (as shown in FIG. 65). The cap members 1038 also include a second coupling portion 1091 configured to provide a snap-fit to a mating coupling feature 1092 on the covers 1036 (see FIG. 75). When the cap members 1038 are moved to the closed position, the cap members 1038 can be snap-fit to the covers 1036 to provide seal to the reaction wells 1030.

The disposable cartridge 1004 can be received in a PCR device (not shown) as described herein for other embodiments. The PCR device can include an optical scanning device and a thermal cycler and any of the features described herein for other embodiments. As discussed previously for other embodiments, the cartridge 1004 can be disposed between a heat plate and an optical scanning device of the PCR device in a vertical orientation such that a read optic can be translated across the reaction wells 1030 of the disposable cartridge 1004.

It is intended that the systems and methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

The PCR systems described herein can provide a variety of system features. For example, some user features include, high resolution color touch screen display, icon based user interface, multiple protocol selection, identification of sample bar coded on cartridge, role-based security authorization, general laboratory user functions, administrator/super user functions, and user identification linked to test and results. Other features include an internal bar code scanner to allow for ability to read and load data from a disposable cartridge, and ability to read ID badge of user or can log-in using a user ID/password. Example hardware features include, rapid heat and cooling (e.g., 5° C./s), power-on self test, high degree of thermal accuracy (e.g., +/−0.1° C.), and internal thermal monitoring. Example software features include, integrated custom logic system, customized protocol by administrator, test results display and export, date and time stamp, system integrity check, thermal cycle confirmation, excitation and emission confirmation, internal emissions monitoring, system performance and error reporting and archive capabilities (internal and external). Some example network peripherals include, wireless and wired networking, high speed USB ports, direct printer output, ability to network multiple devices, data analysis from one centralized computer, data storage and on-line software updates.

### CONCLUSION

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. The specific configurations of the various components can also be varied. For example, the size and specific shape of the various components can be different than the embodiments shown, while still providing the functions as described herein.

What is claimed is:

1. An apparatus, comprising:
a body including a plurality of reaction wells, each of the plurality of reaction wells defining an interior region configured to receive a volume of material to be analyzed, each reaction well from the plurality of reaction wells having a width and a length, the length being greater than the width, each of the plurality of reaction wells including a thermal interface wall bounding a portion of the interior region and a clear wall bounding another portion of the interior region,
the body configured to be inserted in a vertical orientation into a device such that the length of the plurality of reaction wells is disposed vertically, the device configured to detect polymerase chain reactions within the volume of material,
when the body is in the vertical orientation, the clear wall of each of the plurality of reaction wells is disposed such that an optical scanning device can be translated to a plurality of different positions disposed in front of the plurality of reaction wells and detect a fluorescence intensity of the volume of material within each reaction well from the plurality of reaction wells.

2. The apparatus of claim 1, wherein the body includes a base member and a plurality of cover members coupled to the base member, each of the plurality of reaction wells being collectively defined by the base member and one cover member from the plurality of cover members.

3. The apparatus of claim 1, wherein the body includes a base member and a plurality of cover members coupled to the base member, each of the plurality of reaction wells being collectively defined by the base member and one cover member from the plurality of cover members, each cover member from the plurality of cover members including a raised portion defining a fill port, and a portion including the clear wall.

4. The apparatus of claim 1, wherein the body includes a base member, a plurality of cover members coupled to the base member, and a cap member,
each of the plurality of reaction wells being collectively defined by the base member and one cover member from the plurality of cover members, the cap member configured to be coupled to each of the plurality of cover members.

5. The apparatus of claim 1, wherein the body includes a base member, a plurality of cover members coupled to the base member, and a plurality of cap members,
each of the plurality of reaction wells being collectively defined by the base member and one cover member from the plurality of cover members, each cap member from the plurality of cap members configured to be coupled to one cover member from the plurality of cover members.

6. The apparatus of claim 1, wherein the body includes a base member, a plurality of cover members coupled to the base member, and a plurality of cap members,
each of the plurality of reaction wells being collectively defined by the base member and one cover member from the plurality of cover members, each cap member from the plurality of cap members configured to be coupled to one cover member from the plurality of cover members,
each cap member from the plurality of cap members being movable between a closed position and an open position when coupled to the cover members.

7. The apparatus of claim 5, wherein at least a portion of each cap member of the plurality of cap members is made of compressible material.

8. The apparatus of claim 5, wherein at least a portion of each cover member of the plurality of cover members is made of compressible material.

9. The apparatus of claim 1, wherein a depth of the each reaction well is substantially less than the width of the each reaction well.

10. An apparatus, comprising:
a holder member of a polymerase chain reactions (PCR) device configured to receive a cartridge in a vertical orientation, the cartridge including a plurality of reaction wells each defining an interior region containing a volume of material to be analyzed, each reaction well from the plurality of reaction wells having a width and a length, the length being greater than the width, the length being disposed vertically when the cartridge is disposed within the holder member;
a thermal cycler coupled to the holder member, the thermal cycler including a heat plate configured to contact a thermal interface wall bounding a portion of each reaction well from the plurality of reaction wells; and
an optical scanning device coupled to the holder member, the optical scanner configured to translate along an axis substantially perpendicular to an axis defined along the length of each reaction well from the plurality of reaction wells, the optical scanning device configured to measure a fluorescence intensity of the volume of material disposed within each reaction well from the plurality of reaction wells.

11. The apparatus of claim 10, wherein the optical scanning device is configured to measure a fluorescence intensity of the volume of material within each reaction well from the plurality of reaction wells at a plurality of locations within each reaction well form the plurality of reaction wells.

12. The apparatus of claim 10, wherein the optical scanning device is configured to stop at a first location in front of a first reaction well from the plurality of reaction wells for a first time period, measure during the first time period a first fluorescence intensity of the volume of material within the first reaction well, move to a second location in front of the first reaction well, and measure a second fluorescence intensity of the volume of material within the first reaction well during a second time period.

13. The apparatus of claim 10, wherein the optical scanning device is configured to measure the fluorescence intensity of the volume of material within each reaction well form the plurality of reaction wells in real time.

14. The apparatus of claim 10, wherein the cartridge includes a base member and a plurality of cover members coupled to the base member, each of the plurality of reaction wells being collectively defined by the base member and one cover member from the plurality of cover members.

15. The apparatus of claim 10, wherein the cartridge include includes a base member and a plurality of cover members coupled to the base member, each of the plurality of reaction wells being collectively defined by the base member and one cover member from the plurality of cover members, each cover member form the plurality of cover members including a clear wall bounding the interior region, the optical scanning device configured to measure the fluorescence intensity of the volume of material within each reaction well from the plurality of reaction wells through the clear wall.

16. The apparatus of claim 10, wherein the thermal cycler is configured to heat and cool the volume of material within each reaction well from the plurality of reaction wells with a change rate greater than 5° C. per second.

17. A method, comprising:
receiving a cartridge disposed in a vertical orientation within a holder member, the cartridge including a plurality of reaction wells each containing a volume of material to be analyzed, each reaction well from the plurality of reaction wells having a width and a length, the length being greater than the width, the length of the reaction wells being disposed vertically when the cartridge is inserted into the holder member, the device configured to detect polymerase chain reactions within the volume of material in each of the plurality of reaction wells;
cycling a thermal cycler coupled to the holder member to cause a temperature of the volume of material within each reaction well from the plurality of reaction wells to vary;
illuminating with an excitation device the volume of material within a first reaction well from the plurality of reaction wells for a first time period; and
after the illuminating, detecting a fluorescence intensity of the volume of material within the first reaction well from the plurality of reaction wells.

18. The method of claim 17, further comprising:
prior to the illuminating for the first time period, moving the excitation device to a first position disposed in front of the first reaction well, the detecting the fluorescence intensity including detecting the fluorescence intensity of the volume of material within the first reaction well at a first location within the first reaction well.

19. The method of claim 17, further comprising:
prior to the illuminating for the first time period, moving the excitation device to a first position disposed in front of the first reaction well, the detecting the fluorescence intensity including detecting the fluorescence intensity of the volume of material within the first reaction well at a first location within the first reaction well;
moving the excitation device to a second position disposed in front of the first reaction well;
illuminating with the excitation device the volume of material within the first reaction well for a second time period; and
detecting a fluorescence intensity of the volume of material within the first reaction well at the second location within the first reaction well.

20. The method of claim 17, further comprising:
prior to the illuminating for the first time period, moving the excitation device to a first position disposed in front of the first reaction well, the detecting the fluorescence intensity including detecting the fluorescence intensity of the volume of material within the first reaction well at a first location within the first reaction well;
moving the excitation device to a second position disposed in front of a second reaction well from the plurality of reaction wells;
illuminating with the excitation device the volume of material within the second reaction well for a second time period; and
detecting a fluorescence intensity of the volume of material within the second reaction well at a first location within the second reaction well.

21. The method of claim 17, further comprising:
prior to the illuminating for the first time period, moving the excitation device to a first position disposed in front of the first reaction well, the detecting the fluorescence intensity including detecting the fluorescence intensity of the volume of material within the first reaction well at a first location within the first reaction well;
moving the excitation device to a second position disposed in front of a second reaction well from the plurality of reaction wells;
illuminating with the excitation device the volume of material within the second reaction well for a second time period;
detecting a fluorescence intensity of the volume of material within the second reaction well at a first location within the second reaction well;
moving the excitation device to a second position disposed in front of the second reaction well;
illuminating with the excitation device the volume of material within the second reaction well for a third time period; and
detecting a fluorescence intensity of the volume of material within the second reaction well at a second location within the second reaction well.

22. The method of claim 17, prior to the illuminating for a first time period, translating the excitation device along an axis substantially perpendicular to a vertical axis defined by the cartridge to a first position disposed in front of the first reaction well, the detecting the fluorescence intensity including detecting the fluorescence intensity of the volume of material within the first reaction well at a first location within the first reaction well;
translating the excitation device along the axis substantially perpendicular to the vertical axis of the cartridge to a second position disposed in front of a second reaction well; and
illuminating with the excitation device the volume of material within the second reaction well for a second time period; and
detecting a fluorescence intensity of the volume of material within the second reaction well at a first location within the second reaction well.
